(19) United States
(12) Patent Application Publication
(10) Pub. No.: US 2011/0171650 A1
(43) Pub. Date: Jul. 14, 2011

(54) GENE EXPRESSION RELATED TO PREECLAMPSIA

(75) Inventors: Kirk P. Conrad, Gainesville, FL (US); Arundhathi Jeyabalan, Pittsburgh, PA (US); Sandra Anne Founds, Wexford, PA (US); William Allen Hogge, Pittsburgh, PA (US)

(73) Assignees: University of Pittsburgh-Of the Commonwealth System of Higher Education, Pittsburgh, PA (US); University of Florida Research Foundation, Inc., Gainesville, FL (US)

(21) Appl. No.: 13/119,213

(22) PCT Filed: Sep. 16, 2009
(86) PCT No.: PCT/US2009/057103

§ 371 Date: Mar. 16, 2011

Related U.S. Application Data
(60) Provisional application No. 61/097,356, filed on Sep. 16, 2008.

(51) Int. Cl. C12Q 1/68; G01N 33/53

(52) U.S. Cl. 435/6.11; 436/501; 435/7.92

(57) ABSTRACT

Gene expression patterns contemporaneous with early placental development in the first trimester of preeclamptic versus unaffected pregnancies have been obtained. Observation of differences in these gene expression patterns has allowed the identification of biomarkers that are useful in predicting and monitoring preeclampsia. These biomarkers are also useful in screening potential therapeutics for efficacy in the prevention or treatment of preeclampsia.

### CROSS-REFERENCE TO RELATED APPLICATION

This application claims the benefit of U.S. Provisional Application Ser. No. 61/097,356, filed Sep. 16, 2008, the disclosure of which is hereby incorporated by reference in its entireties, including all figures, tables and amino acid or nucleic acid sequences.

### GOVERNMENT SUPPORT

The subject matter of this application has been supported by the Center for Research and Evaluation Faculty Research Funds of the University of Pittsburgh School of Nursing, the American Nurses Foundation, the NIH/NINR Summer Genetics Institute, and grant number NIH PO1 HL30367 Subproject #2. Accordingly, the government has certain rights in this invention.

### BACKGROUND OF THE INVENTION

Preeclampsia (Online Mendelian Inheritance in Man [OMIM] 189800) is a pregnancy-specific disorder that leads among the causes of maternal and infant morbidity and mortality worldwide. Occurring in 4 to 8% of all pregnancies, (Ilekis et al., 2007; ACOG Publication 2002) this multi-systemic disorder contributes to 20% of maternal deaths in the United States alone (Christiansen et al., 2006). Infants are at increased risk of growth restriction and the adverse effects of indicated preterm delivery, which is the only definitive treatment for preeclampsia (Stella et al., 2006). Further burden is imposed by increased risk of cardiovascular disease later in life for women and offspring who survive preeclampsia (Roberts et al., 2003; Sibai et al., 2005).

Prevention, early detection, and specific treatment of preeclampsia are hindered by the fact that the etiology has remained unknown (Sibai et al., 2005). Current consensus implicates placental and endothelial dysfunction, inflammation and genetics in development of preeclampsia (Ilekis et al., 2007; Mohaupt 2007; Nishizawa et al., 2007). Extravillous trophoblasts in preeclamptic pregnancies fail to adequately remodel the maternal uterine spiral arteries, thereby compromising blood flow to the placenta (Ilekis et al., 2007). This alteration reduces placental oxygen and nutrient supply, creating ischemia-hypoxia and oxidative stress associated with reperfusion injury (Khong et al., 1986). This placental pathology leads to the elaboration of a variety of injurious agents into the maternal circulation producing excessive maternal systemic inflammation (Redman et al., 1999) and endothelial dysfunction, and results in potentially lethal sequelae, such as eclamptic seizure, stroke, pulmonary edema, renal failure and hemorrhage (Roberts et al., 1993; Roberts et al., 2002).

A better understanding of the early pathophysiology at the molecular level has been needed. Because preeclampsia resolves with removal of the placenta, (Roberts et al. 2001) microarray studies have been conducted with second and third trimester placental tissue after delivery, in order to examine organ-specific, global genomic variations associated with preeclampsia (Founds et al., 2008). Despite these efforts, the etiology has been elusive because prior microarray analyses in preeclampsia interrogated placentas after disease onset in later gestation, thereby precluding differentiation of cause and effect (Chappell et al., 2006). In contrast, the present invention relates to gene expression patterns early in the course of placental development.

### BRIEF SUMMARY OF THE INVENTION

Gene expression patterns contemporaneous with early placental development in the first trimester of preeclamptic versus unaffected pregnancies have been obtained. Observation of differences in these gene expression patterns has allowed the identification of biomarkers that are useful in predicting and monitoring preeclampsia. These biomarkers are also useful in screening potential therapeutics for efficacy in the prevention or treatment of preeclampsia.

### BRIEF DESCRIPTION OF THE DRAWINGS

FIGS. 1A-1C relate to IPA Networks 1 and 2 involving the 36 genes of interest. The IPA graphics represent genes and potential relationships arranged by cellular compartments. FIG. 1A. Network 1 includes 10 genes from this study related to Cancer, Respiratory Disease, and Cellular Movement. FIG. 1B. Network 2 includes 7 genes from this study related to Inflammatory Disease, Cellular Movement, and Hematological System Development and Function. FIG. 1C. A legend for the IPA Networks of FIGS. 1A and 1B gives meaning of node type symbols and edge type relationships. Dark gray filled symbols represent up-regulated genes of interest from this study. Light gray filled symbols represent down-regulated genes of interest from this study. Unfilled symbols are genes putatively involved in the pathway based on current animal and human studies.

FIGS. 2A-2B. Scatterplots of Perfect Matches only, both with and without case #147. FIG. 2A represents perfect match data with case #147 included. FIG. 2B represents perfect match data with case #147 excluded.

FIG. 3. Plots comparing PM-Only J5, Pooled Variance t, Fold-Change and Random. Efficiency analysis plot comparing the observed overlap at each level of N3 for the PM-alone, untransformed data.

FIG. 4. Naïve Bayes' prediction models' performance. ACE (achieved classification error; =1-accuracy) plot of the performance of Naïve Bayes models with and without sample #147. J5 score is the threshold (cut-off) of the J5 test.

### BRIEF DESCRIPTION OF THE TABLES

Tables 1A-1C. Clinical characteristics of total microarray study sample.

Table 2. Genes differentially expressed in first trimester PE: J5 analysis for CVS microarray.

Table 3. Top Functions and Diseases in IPA associated with high priority genes of interest.

Table 4. Functional groups other than immune incorporating IPA-omitted high priority genes of interest in PE.

Table 5. Genes differentially expressed in first trimester PE: Fold change data for CVS microarray.

Table 6. Hypoxia-regulated genes.

Table 7. Putative HIF-2α regulated genes.

Table 8. J5 and Fold Change Genes that Secrete Proteins into Blood (certain genes have both fold change and J5 values (bold)).

Table 9. Genes differentially expressed in first trimester PE.

### DETAILED DESCRIPTION OF THE INVENTION

In a first aspect, the present invention relates to a method for diagnosing, monitoring, or predicting preeclampsia in a pregnant woman. This method comprises the following steps:

first, quantitatively determining the amount of one or more nucleic acid species in a biological sample obtained from the pregnant woman that hybridize with probe set ID Nos: 205827_at; 215141_at; 202917_s_at; 215733_x_at; 234601_x_at; 227238_at; 239010_at; 214702_at; 1553319_at; 235592_at; 229839_at; 230748_at; 203789_s_at; 226482_s_at; 215388_s_at; 1562053_at; 219911_s_at; 209351_at; 1552858_at; 215108_x_at; 226403_at; 207607_at; 228293_at; 210251_s_at; 1561318_at; 241036_at; 219759_at; 203592_s_at; 205302_at; 1568554_x_at; 1554276_at; 242842_at; 242868_at; 206859_s_at; 204580_at; and 207509_s_at or those set forth in Table 5 on the AFFYMETRIX GeneChip system (Affymetrix, Santa Clara, Calif.; HG-U133 Plus 2.0 GeneChips containing 53,613 probe sets);

second, comparing the amount of the nucleic acid species from the first step to a standard control representing the amount of the nucleic acid species in the corresponding sample from an average non-preeclamptic pregnant woman; wherein an increase and decrease in the amount of the nucleic acid species in the test sample as compared to the standard control indicates preeclampsia or an increased risk of developing preeclampsia. The biological sample is blood, washing from the reproductive tract, urine, saliva, amniotic fluid, or chorionic villus. One aspect of the invention provides for increased expression of nucleic acids that hybridize with 205827_at; 215141_at; 202917_s_at; 215733_x_at; 234601_x_at; and decreased expression of nucleic acids that hybridize with 227238_at; 239010_at; 214702_at; 1553319_at; 235592_at; 229839_at; 230748_at; 203789_s_at; 226482_s_at; 215388_s_at: 1562053_at; 219911_s_at: 209351_at; 1552858_at; 215108_x_at; 226403_at; 207607_at; 228293_at; 210251_s_at; 1561318_at; 241036_at; 219759_at; 203592_s_at; 205302_at; 1568554_x_at; 1554276_at; 242842_at; 242868_at; 206859_s_at; 204580_at; and 207509_s_at.

In some embodiments, the first step can comprise the use of a reverse transcriptase polymerase chain reaction (RT-PCR). In other embodiments, the first step comprises using a polynucleotide hybridization method, or using a primer extension reaction.

Various other embodiments provide a kit for diagnosing, monitoring, or predicting preeclampsia in a pregnant woman. This kit comprises the following: (i) PCR primers for quantitatively determining the amount of one or more nucleic acid species in a biological sample obtained from the pregnant woman, wherein the nucleic acid species hybridize with probe set ID Nos: 205827_at; 215141_at; 202917_s_at; 215733_x_at; 234601_x_at; 227238_at; 239010_at; 214702_at; 1553319_at; 235592_at; 229839_at; 230748_at; 203789_s_at; 226482_s_at; 215388_s_at; 1562053_at; 219911_s_at; 209351_at; 1552858_at; 215108_x_at; 226403_at; 207607_at; 228293_at; 210251_s_at; 1561318_at; 241036_at; 219759_at; 203592_s_at; 205302_at; 1568554_x_at; 1554276_at; 242842_at; 242868_at; 206859_s_at; 204580_at; and 207509_s_at or those set forth in Table 5 on the AFFYMETRIX GeneChip system (Affymetrix, Santa Clara, Calif.; HG-U133 Plus 2.0 GeneChips containing 53,613 probe sets) and (ii) a standard control representing the amount of the nucleic acid species in the corresponding sample from an average non-preeclamptic pregnant woman.

Another aspect of the invention relates to a method for diagnosing, monitoring, or predicting preeclampsia in a pregnant woman using immunoassay(s). This method comprises the following steps:

first, quantitatively determining the amount of one or more polypeptide species in a biological sample (test sample) obtained from the pregnant woman that bind to specific antibodies, said one or more polypeptide species being set forth in Table 8;

second, comparing the amount of said one or more polypeptide species identified in the first step to a standard control representing the amount of the corresponding polypeptide species in a corresponding sample from an average non-preeclamptic pregnant woman (a control sample or standard control); wherein an increase (overexpression) and decrease (underexpression) in the amount of said at least one polypeptide species in the test sample as compared to the standard control indicates preeclampsia or an increased risk of developing preeclampsia. The biological sample is blood, serum, plasma, endometrial tissue, washing from the reproductive tract, urine, saliva, cerebral spinal fluid, amniotic fluid, or chorionic villus. The woman being examined is examined during the first trimester of gestation. In other embodiments, the woman is during the second or third trimester of gestation.

As discussed above, immunoassays can be used to detect at least one secreted protein disclosed in Table 8, the expression levels of said at least one secreted protein, and comparison of said at least one secreted protein to a control (standard control) sample. Protein expression (secretion) can be detected by any suitable method, such as gas chromatography-mass spectrometry. In some embodiments, proteins are detected by immunoassays.

Antibody binding is detected by techniques known in the art (e.g., radioimmunoassay, ELISA (enzyme-linked immunosorbant assay), “sandwich” immunoassays, immunoradiometric assays or Western blots. In some embodiments, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention. In aspects of this invention, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. In other embodiments, the immunoassay described in U.S. Pat. Nos. 5,599,677 and 5,672,480; each of which is herein incorporated by reference.

In various aspects of the invention, the woman being examined is examined during the first trimester of gestation. In other embodiments, the woman is during the second or third trimester of gestation.

The term “nucleic acid” can be understood to mean, according to the present invention, either a double-stranded DNA, a single-stranded DNA or products of transcription of the said DNAs (e.g., RNA molecules).

The term “preeclampsia” as used herein refers to a condition that occurs during pregnancy, diagnosed by the new onset of high blood pressure accompanied by the presence of proteins in the urine and may include edema (swelling). Preeclampsia, sometimes called toxemia of pregnancy, is related to a more serious disorder called “eclampsia”, which is preeclampsia together with seizure. These conditions usually develop during the second half of pregnancy (after 20 weeks), though they may develop shortly after birth or before 20 weeks of pregnancy.

The term “primer extension reaction” as used herein refers to any polymerization process mediated by the action of a nucleotide polymerase, e.g., a DNA polymerase, by extending a predetermined polynucleotide sequence that is at least partially complementary to a template sequence under appropriate conditions.

Probe set ID Nos: 205827_at; 215141_at; 202917_s_at; 215733x_at; 234601_x_at; 227238_at; 239010_at; 214702_at; 1553319_at; 235592_at; 229839_at; 230748_at; 203789_s_at; 226482_s_at; 215388_s_at; 1562053_at; 219911_s_at; 209351_at; 1552858_at; 215108_x_at; 226403_at; 207607_at; 228293_at; 210251_s_at; 1561318_at; 241036_at; 219759_at; 203592_s_at; 205302_at; 1568554_x_at; 1554276_at; 242842_at; 242868_at; 206859_s_at; 204580_at; and 207509_s_at on the AFFYMETRIX GeneChip system (Affymetrix, Santa Clara, Calif.; HG-U133 Plus 2.0 GeneChips containing 53,613 probe sets), as used herein, refer to nucleic acid sequences found on the aforementioned AFFYMETRIX GeneChip system. The polynucleotide sequences are identified by database accession numbers (e.g., NM_{—}003394.1, etc.) in Tables 2, 5 and 9 and each of the accession numbers are hereby incorporated by reference in their entireties.

“Standard control” or “control sample” as used herein refers to a sample suitable for use in a method of the present invention, e.g., in order for quantitatively determining the amount of a nucleic acid. Such a sample contains a known amount of the nucleic acid that closely reflects the average level of the nucleic acid in an average non-preeclamptic pregnant woman. Similarly, a “standard control” may be derived from an average healthy non-pregnant woman.

“An increase and decrease in the amount of the nucleic acid or polypeptide species in the test sample as compared to the standard control” refers to a positive or negative change in amount from the standard control. An increase is preferably at least 2.00 fold, 2.25 fold, 2.50 fold, 2.75 fold, 3.00 fold, 3.25 fold, 3.5 fold, 3.75 fold, 4.00 fold, 4.25 fold, 4.50 fold, 4.75 fold, of 5.00 fold. Similarly, a decrease is at least 2.00 fold, 2.25 fold, 2.50 fold, 2.75 fold, 3.00 fold, 3.25 fold, 3.5 fold, 3.75 fold, 4.00 fold, 4.25 fold, 4.50 fold, 4.75 fold, of 5.00 fold. For example, an increase of 2+ or greater or −2 or below would be considered significant difference from control. These expression levels (+2 or −2) can also be referred to as “overexpressed”/“overexpression” or “underexpressed”/“underexpression”.

A “polynucleotide hybridization method” as used herein refers to a method for detecting the presence and/or quantity of a polynucleotide based on its ability to form Watson-Crick base-pairing, under appropriate hybridization conditions, with a polynucleotide probe of a known sequence. Examples of such hybridization methods include Southern blotting and Northern blotting.

“PCR primers” as used herein refer to oligonucleotides that can be used in a polymerase chain reaction (PCR) to amplify a nucleotide sequence originating from a nucleic acid (RNA transcript). Some aspects of the invention provide for primers that comprise the sequences of probe set ID Nos: 205827_at; 215141_at; 202917_s_at; 215733_x_at; 234601_x_at; 227238_at; 239010_at; 214702_at; 1553319_at; 235592_at; 229839_at; 230748_at; 203789_s_at; 226482_s_at; 215388_s_at; 1562053_at; 219911_s_at; 209351_at; 1552858_at; 215108_x_at; 226403_at; 207607_at; 228293_at; 210251_s_at; 1561318_at; 241036_at; 219759_at; 203592_s_at; 205302_at; 1568554_x_at; 1554276_at; 242842_at; 242868_at; 206859_s_at; 204580_at; and 207509_s_at on the AFFYMETRIX GeneChip system (Affymetrix, Santa Clara, Calif.; HG-U 133 Plus 2.0 GeneChips containing 53,613 probe sets). Various combinations of the aforementioned primers can be included in a primer kit as set forth herein.

As used in this specification and the appended claims, the singular forms “a”, “an”, and “the” include plural reference unless the context clearly dictates otherwise. Additionally, the terms “comprising”, “consisting of” and “consisting essentially of” are defined according to their standard meaning. The terms may be substituted for one another throughout the instant application in order to attach the specific meaning associated with each term. The phrases “isolated” or “biologically pure” refer to material that is substantially or essentially free from components which normally accompany the material as it is found in its native state. Thus, isolated peptides or nucleic acids, in accordance with the invention, preferably do not contain materials normally associated with the peptides in their in situ environment.

### Materials and Methods

### A. Samples

Chorionic villus sampling (CVS) was performed as follows. Samples were donated from 2001-2005 by women who gave informed consent for research participation through procedures approved by the University of Pittsburgh and Magee-Women's Hospital Institutional Review Boards. Consent for the research was obtained by genetics counselors during the process of informed consent for the clinical CVS procedure. A physician specialized in CVS (W. A. H.) obtained specimens for clinical cytogenetics by aspiration of tissue into a 20 cc syringe containing Amniomax solution for cytogenetics cell culture (Invitrogen, Carlsbad, Calif.). The solution was not expected to affect RNA in any way, although experiments to test this opinion have not been performed (T. Jackson, Invitrogen; personal communication, Feb. 28, 2008). As part of the routine CVS procedure, the clinical specimen was poured from the syringe into a Petri dish to be assessed by the clinician for adequacy of amount aspirated (at least 25 mg of villi). No extra tissue was extracted for the research. If surplus tissue not needed for clinical analyses was available, villi grossly free of decidua and maternal blood were removed from the Amniomax in the Petri dish, placed in an Eppendorf tube, and snap-frozen in less than 10 minutes of CVS aspiration from the patient. Research specimens were stored at −80° C. for analyses after birth outcomes became available. Frozen samples occupied approximately 5-30 μl in the Eppendorf tubes. Eighty percent of all 160 consented participants had surplus CVS tissue for the research study. The rate of preeclampsia in the cohort was ˜3%.

Certain preeclampsia (PE) and control (C) samples were chosen for study by microarray. Each of 4 CVS specimens from women who subsequently developed preeclampsia (PE) was matched based on parity, gestational age at CVS within 3 days, and race with 2 unaffected control (C) specimens for the microarray analysis. The sample size of 4 PE patients was determined by availability of samples in the CVS specimen bank meeting the study's diagnostic criteria and was the minimal number needed for statistical variance. PE was defined as new onset of hypertension and proteinuria after 20 weeks gestation with blood pressure ≧140 and/or 90 on at least 2 occasions at least 6 hours apart, and ≧300 mg of protein in a 24 hour urine (Gifford, et al., 2000). These women did not have underlying medical disorders or other obstetrical complications. Controls were defined as specimens from normotensive women with blood pressure <140/90, no proteinuria, and without other pregnancy complications or underlying medical disorders.

Additional samples (AS) for analysis by quantitative real time polymerase chain reaction (qRT-PCR) were prepared to test replication of the microarray findings. Twenty-four stored specimens were selected from women without pregnancy complications or underlying medical disorders. Matching AS to the PE cases was prioritized by parity, gestational age at CVS and race. Parity was limited to ≦3 pregnancies and gestational age at CVS within 3 days.

Clinical data analysis was carried out. Demographic and clinical characteristics of participants were analyzed to examine underlying assumptions of group assignment. PE (N=4) and C (N=8) samples submitted for microarray procedures were compared using distributionally appropriate t-test or chi square with p≦0.05 set as level of significance. Additional samples (“AS”, N=24) selected for replication purposes were compared with PE and C groups using ANOVA and Bonferroni adjustment with p≦0.05 set as level of significance (SPSS 15.0, Carey, NC).

Total RNA extraction and microarray procedures were conducted at the University of Pittsburgh Genomics and Proteomics Core Laboratory (GPCL). Specialized methods were applied for the particular tissue type, resulting in good RNA integrity (Agilent RIN≧6.0). In detail, the procedures were as follows: Each frozen CVS specimen was homogenized in 1 ml of TRIzol (Invitrogen, Carlsbad, Calif.) using a Polytron mechanical disrupter (Glen Mills, Clifton, N.J.) in less than 1 minute. Samples were incubated for at least 5 minutes at room temperature to allow complete dissociation. Two-tenths volume chloroform was then added. The reaction was mixed vigorously and allowed to incubate 3 minutes at room temperature. Following phase separation by centrifuging for 15 minutes at 12,000×g at 2-8° C., the aqueous phase was transferred to a clean microcentrifuge tube. The RNA was precipitated by adding 0.5 ml isopropyl alcohol, incubating for 5 minutes at room temperature and, finally, spinning 15 minutes at 12,000×g at 2-8° C. The supernatant was removed and the pellet washed by adding 1 ml 75% ethanol and spinning 5 minutes at 7,500×g at 2-8° C. The supernatant was carefully removed and the pellet was allowed to air dry for 5 minutes. The RNA was resuspended in nuclease free water. RNA integrity was evaluated on an Agilent Bioanalyzer (RIN≧6.0; Agilent, Santa Clara, Calif.). Samples were stored at −80° C.

### B. Microarray Data Collection

The Affymetrix GeneChip system (Affymetrix, Santa Clara, Calif.) was used for microarray analysis with HG-U133 Plus 2.0 GeneChips containing 53,613 probe sets. The GPCL facility conducted the analysis according to manufacturer's instructions as explained in more detail below.

Microarray data collection was as follows: Two and one-half micrograms (μg) of total RNA was used as template in a reverse transcription reaction using oligo(dT) 24 primers attached to a T7 RNA polymerase promoter sequence. This single stranded cDNA was transformed into double stranded cDNA. Ten units of T4 DNA Polymerase were added and the reaction incubated at 16° C. for an additional 5 minutes. The reaction was stopped by the addition of EDTA to 0.03 M and cleaned up using an Affymetrix cDNA clean up column. At the end of the second strand reaction, the cDNA sample was mixed with DNA binding buffer and this mixture applied to the column. The column was spun in a microfuge to bind the cDNA to the membrane. DNA wash buffer supplied with the kit was used to wash the membrane, which was then dried by centrifugation. The cDNA was eluted with provided elution buffer. An aliquot of the ds-cDNA equivalent to 5-7 μg of starting RNA was added as template to an in vitro transcription reaction as per the Affymetrix IVT labeling kit. The resulting biotinylated cRNA was purified using an Affymetrix RNA clean up column. The procedure was identical to that for the DNA clean up using appropriately modified membranes and buffers as supplied. After elution the cRNA was quantified by reading the OD₂₆₀ of a 1:100 dilution on a spectrophotometer. In addition, 1 μl of the cRNA was run on an Agilent Bioanalyzer to verify that most product represented full size transcripts. An aliquot of 20 μg of cRNA was incubated at 94° C. in fragmentation buffer (40 mM Tris-Acetate pH 8.1, 100 mM KOAc, 30 mM MgOAc) for 35 minutes to break the RNA into segments of 35 to 200 bases. A 1 μl aliquot of the sample was run on an Agilent Bioanalyzer to verify that fragmentation resulted in RNA of the desired size distribution. Fifteen μg of the fragmented RNA was added to a final volume of 300 μl hybridization cocktail consisting of 1× hybridization buffer, 100 μg/ml Herring sperm DNA, 50 μg/ml Acetylated BSA, 50 pM Affymetrix Control Oligo B2, 1× Affymetrix Eukaryotic Hybridization Control. 1× hybridization buffer contains 100 mM MES, 1M Na+, 20 mM EDTA, 0.01% Tween 20.

An appropriate volume of this sample was applied to HG-U133 Plus 2.0 GeneChip and incubated overnight at 45° C. with rotation. Following hybridization, the sample was removed and the GeneChip cassette filled with non-stringent wash buffer. The chip was loaded onto an Affymetrix 450 Fluidics station for wash and stain. Wash and stain protocols are the double stain protocols developed by Affymetrix for use with the Affymetrix 450 Fluidics Station. To remove unbound sample, arrays were first washed with non-stringent wash buffer followed by a stringent wash in 100 mM MES, 0.1M Na+, 0.01% Tween 20. The GeneChips were then stained for ten minutes in streptavidin-phycoerythrin (SAPE) solution. Non-stringent buffer was used to wash off the first stain solution. Signal amplification was achieved by ten minutes incubation with biotinylated anti-streptavidin, followed by a second ten minute incubation with SAPE. The chip was washed and filled with non-stringent wash buffer before being removed from the fluidics station and scanned using the GeneArray 3000 scanner.

The Affymetrix GeneChip system uses a photolithographic process to manufacture 25-mer oligonucleotide probes directly on the array surface. Each mRNA or EST sequence is represented by 11 probe pairs (the probe set for this gene). Each probe pair consists of one feature containing a perfect match probe (PM) and an adjacent feature containing a mismatch probe (MM). The sequences of the two probes differ by one base in the central position.

Data acquisition from the microarray was via accepted methods. Gene expression intensities were derived from the .cel files using dchip (Li et al., 2001) and BRB-Arrays Tools (Simon et al., 2007). The Affymetrix HG-U133 Plus 2.0 GeneChip contains 53,613 probe sets.

QA/QC analysis of the data was carried out. Examination of the scanned images revealed a number of potential outliers in the array from patient #147. Subsequent data quality assurance/quality control (QA/QC) analysis by correlation and multiplicative-additive (M-A) plots confirmed that the inclusion of PE sample #147 led to group-wise high-expression outliers. All analyses were conducted including and excluding #147 for further assessment.

Normalization was conducted as follows. The data were represented as PM only, ‘Signal’ (log 2 (PM-MM)), and Robust Multi-array Average (RMA) normalized in BRB-Array Tools (Simon et al., 2007). Alternative normalization methods were also conducted using the online program for Cancer Gene Expression Data Analysis (caGEDA) (Patel 2004).

### C. Identification of Differentially Expressed Genes

Following data quality assurance/quality control (QA/QC) and normalization procedures, differentially expressed genes were identified using the J5 test, Pooled Variance t-test (PVT) and Fold Change 3 (FC=[mean 1−mean 2]/mean 2) as implemented in the online program for Cancer Gene Expression Data Analysis (caGEDA) (Patel 2004). The J5 test is a gene-specific ratio between the mean differences in expression intensity of a gene in two groups to the average mean group difference of all m genes for the entire data set. These alternative methods were evaluated using QA/QC plots and ‘efficiency analysis,’ as implemented in caGEDA. In efficiency analysis, the data are split into two random subsets of roughly equal size. Both data sets are analyzed separately, yielding the number of genes found to be significant at each point over the range of the threshold of each test using the two data sets (N1 and N2, respectively). The degree of overlap is compared among tests as the number of genes in the overlap (N3) varies. Absent artifacts, the method that provides the highest internal consistency (highest degree of overlap) is preferred over methods that fail to yield internally consistent gene lists. QA/QC of sample #147 included many outliers. The genes found to be differentially expressed with the most efficient test, whether #147 was included or excluded, were considered further. Efficiency analysis was performed over the range of each test with 30 iterations.

D. Prediction modeling

Naïve Bayes prediction models were evaluated using leave-one-out cross-validation (LOOCV) in caGEDA (Patel 2004) with the J5 test used for feature selection. LOOCV refers to splitting the dataset in two, one group is a learning set and the other a test set. ‘Learning’ or classifier construction is conducted on the training set and evaluated in the test set. Modeling was attempted, both including and excluding array #147. For this step, the J5 test was wrapped within the evaluation loop (an iterative function within the computer program) to minimize the possibility of overtraining, the bias created when classifiers are built from the same dataset to be predicted. The models with lowest achieved classification error (ACE) found at the highest threshold (smallest number of genes) were preferred.

### E. Functional Analysis

Functional analysis of identified differentially expressed genes was carried out. Annotations were retrieved using a Batch Query given the Probe Set Identification numbers (Probe IDs) submitted to the Affymetrix NetAffx resource (NetAffx. 2007). Probe IDs and J5 scores were also submitted to Ingenuity Pathways Analysis 5.5 bioinformatics software (IPA) (Ingenuity 2007) for further investigation of known genes, molecular networks and functions. The Functional Analysis of a Network within IPA identified the biological functions and diseases that were most significantly related to the genes in the Network. The Network genes associated with biological functions and diseases in the Ingenuity Pathways Knowledge Base (IPKB) were analyzed within IPA by Fisher's exact tests to calculate a p-value, determining the probability that each biological function and/or disease assigned to a Network is due to chance alone, with p-value set at 0.05. IPA Functional Analysis has three primary categories of functions: Molecular and Cellular Functions, Physiological System Development and Function, and Diseases and Disorders. Eighty-five high level functional categories are classified under these primary categories. Lower level functions are classified within the high level categories. Specific functions are the lowest level functions found in IPA. Each lowest level function has a population of associated genes. Specific functions are drawn from manually curated animal and human research findings stored within the IPKB. Lower level functions and specific functions may be classified within multiple high level categories.

### F. Quantitative RT-PCR

Quantitative real-time polymerase chain reaction (qRT-PCR) was conducted on a subset of genes to validate the microarray findings. cDNA was generated from 0.1 μg of total mRNA using the high capacity cDNA reverse transcription kit (Applied Biosystems [ABI]; Foster City, Calif.). The most appropriate endogenous control for this tissue was determined using the endogenous control plate (ABI). Ribosomal protein large P0 (RPLP0 [OMIM 180510]; ABI assay #Hs99999902) was selected and utilized as the endogenous control for each sample. The samples utilized for qRT-PCR included the original 12 microarray samples (PE & C) as well as 24 additional samples (AS) from unaffected pregnancies. Each gene was evaluated in duplicate for each sample. Three genes were chosen for evaluation due to being the highest up- or down-regulated in the microarray findings. Pre-designed probe and primer sets were utilized for the CCK gene (ABI assay #Hs00174937), CTAG2 gene (ABI assay # Hs00535628) and LAIR2 gene (ABI assay # Hs00430498); gene names and OMIM identification numbers appear in Table 2. qRT-PCR was conducted according to routine protocols using the ABIPRISM 7000 system (ABI).

Data analysis for qRT-PCR was conducted. Raw cycle threshold (C_{T}) values were determined using SDS1.1 software (ABI). Relative gene expression was determined using the comparative C_{T} method. A threshold value of 0.01 was used for LAIR2, 0.06 for CTAG2, and 0.16 for CCK. The average value for each sample was normalized to the average value of endogenous housekeeping gene RPLPO in the same well, resulting in ΔC_{T}. (Dharmaraj 2007) ΔC_{t, reference} was the Cₜ value for the calibrator, normalized to RPLPO. Sample #38 was used as the calibrator for CCK and LAIR2 because it was a C specimen from a nulliparous nonsmoker at 11.0 weeks, an average gestational age at CVS. Sample #138, also a nulliparous nonsmoker at 11.0 weeks, was used as a calibrator for CTAG2 because there were undetermined values for #38. The difference between ΔCₜ and the average calibrator expression value resulted in ΔΔCₜ. 2^{(-ΔΔCt)} determined the fold change in expression level relative to the calibrator sample. Fold changes were analyzed by Kruskal-Wallis (KW) with significance set at p<0.05. Analyses were carried out in Excel and SAS 9.1 (SAS Institute Inc., Cary, N.C.).

### G. Fold Change Analysis

Secondary analysis was conducted by expression fold changes (FC; Table 3). Scatterplots demonstrated that use of untransformed raw data was appropriate. The average expression level of each transcript for all cases and all controls were calculated. For pairs with average expression level of at least 30 in both cases and controls, the ratio of means in PE to C was calculated as the fold ratio. Fold ratio over 0 are analogous to fold change. Fold ratio below 0 is expressed as a decimal number, which is converted to fold change by dividing 100 by the numeric following the decimal (x⁻ⁿ). Fold change greater than +2 or less than −2 was considered significant (Patel 2004).

### Results:

Clinical data are summarized in Table 1A. All women in the sample for microarray analysis (total N=12) were of advanced maternal age ≧35 years (35-44), White, and had normal fetal karyotype on CVS. PE (N=4) and C (N=8) groups did not differ in maternal age (p=0.34; Table 1A). Eleven of 12 samples were from nulliparous women and one C was a multiparous participant in her second pregnancy; groups did not differ by parity (p=1.00). Although not significantly different, BMI showed a trend toward significance with PE BMI 29.9±4.2 and C BMI 24.5±4.0 (mean±SD; p=0.06). Gestational age at CVS of both PE and C groups ranged from 10.7-12.4 weeks and did not differ between groups (p=0.78). PE and C groups did not differ in gestational age at delivery (p=0.11), birthweight (p=0.10), or infant sex (p=1.00). One C participant reported smoking; all others were self-reported nonsmokers. Groups did not differ in smoking (p=1.00).

Additional clinical data for the four preeclampsia cases are shown in Tables 1B and 1C. The four PE participants met inclusion criteria for hypertension and proteinuria. Three were hyperuricemic for gestational age (Table 1C). Systolic and diastolic blood pressures at less than 20 weeks showed no preexisting hypertension. Participant #147 reached a severe blood pressure above 160/110 and was delivered by cesarean section. Participant #19 had low platelets, high creatinine, and was also delivered by cesarean section. Two PE cases (#21 and #147) delivered preterm, prior to 37 weeks' gestation. PE #21 was obese (BMI≧30) and her infant was growth restricted, below 10^{th} percentile for gestational age, delivered by pitocin induction.

Participants in the AS group ranged in age from 34-45 years (mean 38.2 years) and were 10.4-13.0 weeks gestation (mean 11.5 weeks) with BMI 19.5-36.4 (mean 25.6) at CVS. Seven were nulliparous and 17 were multiparous women with uncomplicated pregnancies and unknown CVS gene expression patterns. All were White women and had normal fetal karyotype on CVS. All were self-reported nonsmokers. It is unknown whether each multiparous woman conceived with the same partner for all of her pregnancies. The AS group did not differ from PE or C by maternal age (p=0.59), gestational age at CVS (p=0.66), or BMI (p=0.19) at CVS.

Oligonucleotide microarray analysis of CVS specimens by the methods described herein produced global gene expression patterns in early pregnancies destined for preeclamptic versus unaffected outcomes. Four PE compared with 8 C specimens resulted in a set of 36 differentially expressed genes.

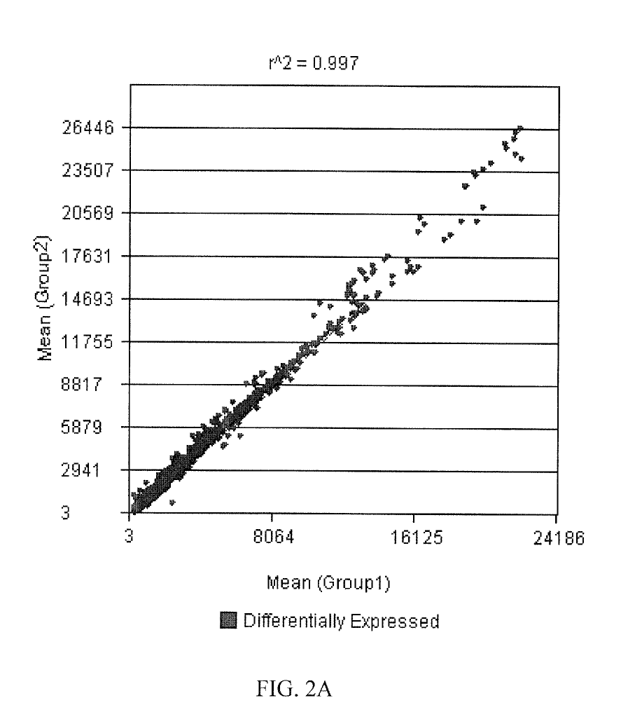

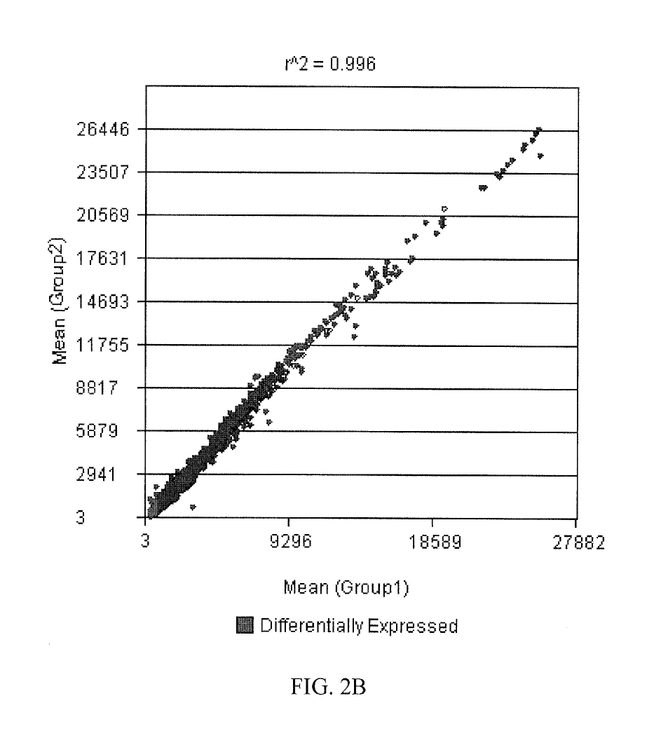

The QA/QC correlogram scatterplots comparing signal intensity across the microarrays were prepared that included case #147 (FIG. 2A) or excluded #147 (FIG. 2B). High-expression outliers found in #147 resulted in subsequent analyses being performed twice, alternatively including or excluding #147. Robust Multi-array Average (RMA) normalization in RMA-BRB-Array Tools (Simon et al., 2007) resulted in a coefficient of variation of 0.001.

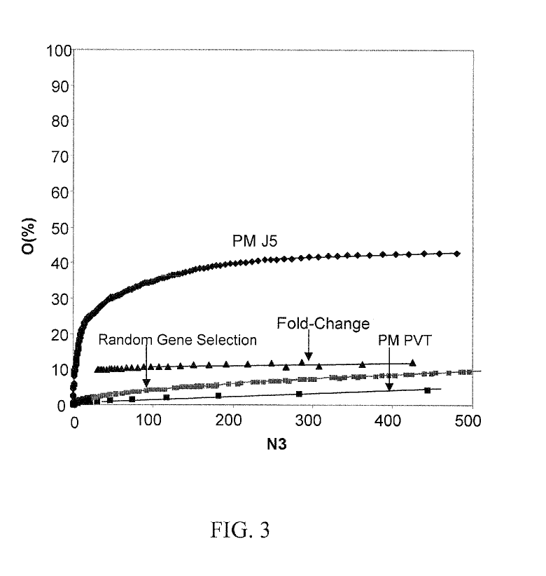

Expression levels were assessed using the J5 test, PVT and FC as implemented in caGEDA (Patel 2004). The J5 test using the raw Perfect Match data (Supplement) only led to the most efficient and internally consistent set of differentially expressed genes, exhibiting 40% consistency in the set of genes found to be differentially expressed between PE and C (FIG. 3). This contrasts with less than 5% internal consistency explained by PVT and approximately 10% explained by FC. Fold change and the t-test are known to lead to high false-positive rates, especially with small sample number (Patel 2004). Efficiency analysis in J5 identified 36 genes of interest in the overlap of gene sets with and without #147.

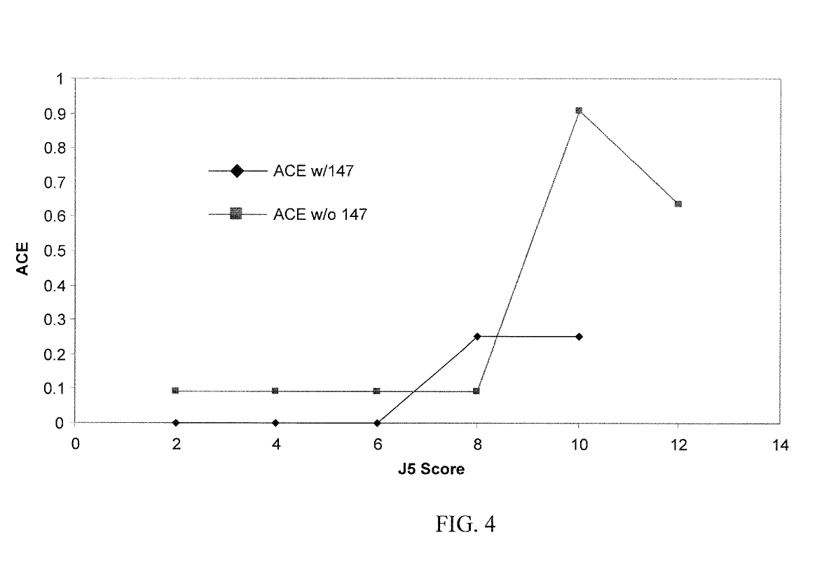

Prediction modeling was explored with Naïve Bayes models using LOOCV (FIG. 4). Modeling was conducted with and without sample #147. The modeling that included #147 exhibited perfect accuracy, equaling sensitivity and specificity (SN=SP=ACC=1.0). The model with a J5 score of 8, which excluded #147, led to the lowest ACE for potential genomic biomarkers of preeclampsia with 90% accuracy.

The Affymetrix probe identification numbers (probe IDs) submitted to NetAffx resulted in annotations for gene names and Gene Ontology (GO) Molecular Function Description for the 36 genes of interest (Tables 2 and 9; Table 9 includes OMIM numbers, gene names and symbols). Five genes of interest were up-regulated and 31 were down-regulated. At this time, 7 are mapped genes with unknown functions and 4 are unmapped with unknown function. NetAffx identified GO Pathway information for only 3 of the 36 genes of interest (Table 9).

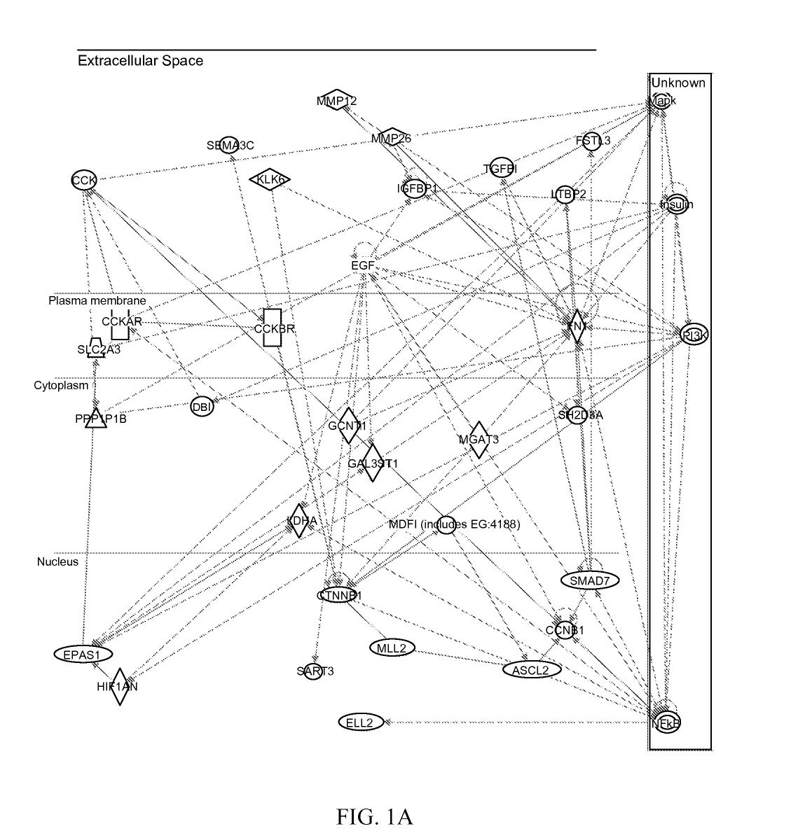

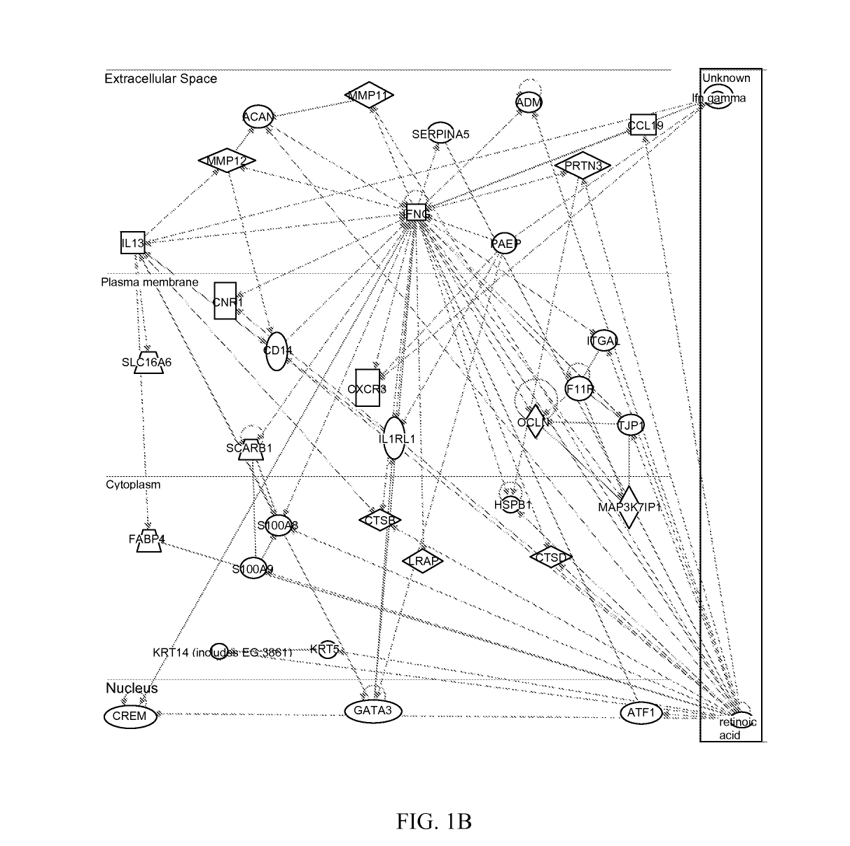

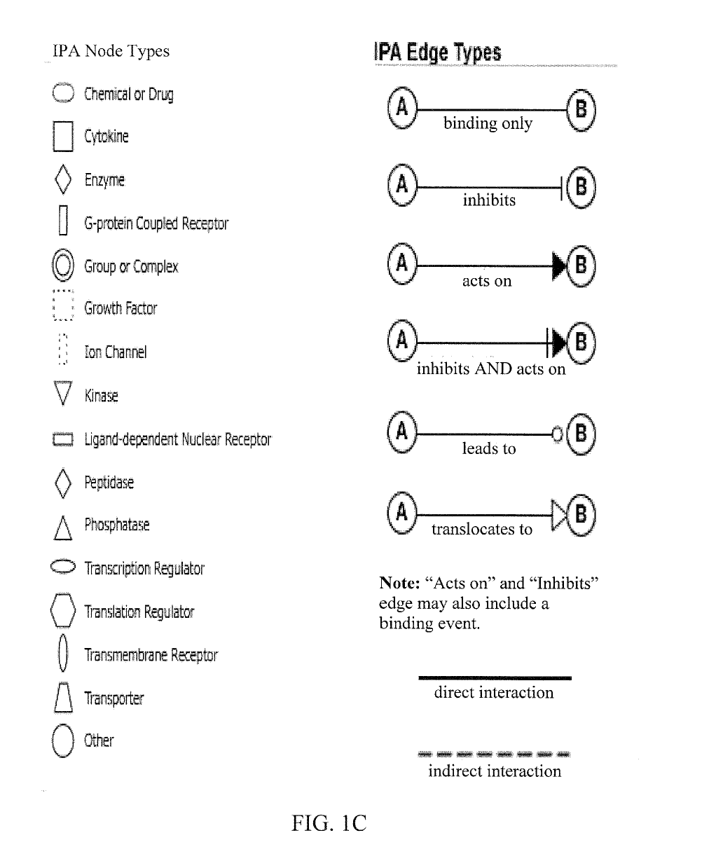

IPA identified potential relationships among some of the genes of interest. Two Networks were developed from the 36 genes imputed with other genes associated through previous studies annotated in the IPKB (FIG. 1). Top Functions and Diseases associated with Network 1 involving 10 of the 36 genes of interest were Cancer, Respiratory Disease, and Cellular Movement. Top Functions and Diseases associated with Network 2, involving 7 genes of interest, were Inflammatory Disease, Cellular Movement, and Hematological System Development and Function. MMP12 was the only gene shared between Networks 1 and 2.

Seventy-two Function and Disease categories among the genes of interest reached significance (p<0.05). Ten functional categories achieved the highest significance levels and included more than one of the 36 genes of interest (Table 3). Categories comprising the 2 Networks among the genes of interest included lower level function subcategories. Lower level functions within Cancer were cancer, neoplasia, tumorigenesis, ovarian cancer, gonadal tumor, mitosis, cell rounding, invasion, apoptosis, adhesion, migration, attachment, and detachment. Respiratory Disease was the 28^{th} most significant Function and Disease category which included FN1, MMP12, CCK, and EPAS1 (−log p-value=1.63E-03-2.72E-02). Lower level functions within Respiratory Disease included primary pulmonary hypertension, lung tumor, lung cancer, adhesion, neonatal surfactant, and emphysema. Cellular Movement included migration of all types of leukocytes, blood, intestinal, embryonic, neuronal, bone marrow, gonadal and cancer cells, cell movement, immobilization, scattering, and invasion. Inflammatory Disease was the 62^{nd} most significant Function and Disease category which included F11R, S100A8, and MMP12 (−log p-value=8.24E-03-2.42E-02) and lower level functions related to juvenile rheumatoid arthritis and emphysema. Hematological System Development and Function included migration and cell movement of all types of leukocytes, cell spreading, adhesion, immobilization, replacement, and proliferation.

Relative quantitation of LAIR2, CCK, and CTAG2 compared to calibrator sample was conducted by qRT-PCR. LAIR2 was significantly down-regulated by a median fold change of 0.21 in PE, compared to median fold change 1.17 in C, and 4.99 in AS (p=0.0004). The median fold change in CCK was 25.37 (range 1.01-51.45) in PE, compared to 2.19 (0.46-30.38) in C, and 5.77 (0.07-26.63) in AS. The raw microarray expression level and qRT-PCR fold change in each PE sample were 4223.4 and 51.45 in #19, 732 and 1.72 in #21, 7339.4 and 49.01 in #58, 59.7 and 1.01 in # 147. Although the trend for CCK fold change was in the same direction as the microarray overexpression, the qRT-PCR differences by Kruskal-Wallis were not significant between groups (p=0.60). qRT-PCR for CTAG2 resulted in 11 of 36 samples as “undetermined.” The values for endogenous control RPLPO were consistent among all 36 samples for each of the 3 genes in qRT-PCR with an average of 24.25±0.12 in CTAG2. The median fold change in CTAG2 was 5.78 (range 0.28-11.27) in PE, compared to 0.04 (0.01-1.00) in C, and 0.90 (0.02-12.64) in AS. The trend in CTAG2 fold change was toward overexpression in PE, but the differences were not significant (p=0.39).

### Discussion:

This microarray analysis of surplus CVS produced differences in global gene expression in placentas of early pregnancies destined for preeclampsia. Utilization of first trimester placentas with known pregnancy outcomes, oligonucleotide genechips, and subsequent prediction modeling distinguish our study from previous placental microarray investigations in preeclampsia (Founds et al., 2008) qRT-PCR confirmed the trends in over- and underexpression in the microarray analysis. The variation in CTAG2 qRT-PCR expression patterns may indicate methylation in nonexpressors denoted as “undetermined” among all 3 groups of samples (Lethe et al., 1998). CCK was also up-regulated 3.1 fold in placentas of preeclamptic pregnancies at 29-32 weeks in a previous study (Reimer el al., 2002). Overall, our results directly support the concept of the placental origins of the disorder (Redman et al., 2005) and allow for targeted investigation of placental-derived biomarkers in early pregnancy. Assessment of cause rather than effect of preeclampsia is likely to have been more discernable in the first trimester placental tissues.

The findings in this study suggest that impaired placentation in preeclampsia may be associated with an overall deficiency rather than an excess of gene expression, insofar as 31 of the 36 genes of interest were down-regulated. Preconceptional testing of susceptibility to preeclampsia could be developed from variants of the genes of interest. In addition, several produce secreted protein (FIG. 1), such that measurement of one or a combination of these biomarker proteins in maternal blood in the first trimester may prove to be a predictive screening test for preeclampsia.

Innate immune responses at the maternal-fetal interface are likely to be represented by our genes of interest. Remarkably, 12 of the 36 genes, 7 not previously associated with preeclampsia, are involved in immune dysregulation (Table 9). All of the immunoregulatory genes except S100A8 were down-regulated, implicating deficient, blocked, or impaired function. LAIR2, HPS3, and SART3 are immune-related genes (Table 9) that were not incorporated by IPA into the immune pathway (FIG. 1; Table 3). The immune dysregulated cells may be trophoblasts, which are fetoplacental epithelial cells (Petty et al., 2006) that act as a pregnancy-specific component of the innate immune system (Guleria et al., 2000). By day 14 post conception, cytotrophoblasts (CTBs) have breached the chorionic basement membrane, switching from a proliferative to an invasive phenotype as extravillous trophoblasts (EVTs) (Huppertz et al., 2007). Lower level functions in Networks 1 and 2, e.g., inflammation, migration, and invasion, are involved in CTB placentation processes (FIG. 1). The EVTs faun cell columns contacting maternal immune cells in the decidua (Benirschke et al., 2006). From these columns, EVTs invade the uterine wall and remodel the maternal spiral arteries by displacing smooth muscle and endothelial cells (Pijnenborg et al., 1983). Normal trophoblast development differs from cancer in that proliferation ceases during invasion (Huppertz et al., 2007). Various genes associated with both of these processes were down-regulated in preeclampsia (Tables 2 and 9). In the current analysis, no notable differential expression existed between PE and C in EVT epithelial or integrins (Damsky et al., 1992; Irving et al., 1995) or human leukocyte antigens (Apps et al., 2007) identified in other studies as dysregulated in CTBs of later gestation. Alternatively, some of the differentially expressed immunoregulatory genes may suggest abnormalities of fetoplacental Hofbauer cells, which are macrophages that populate the villous core (Benirschke et al., 2006). The maternal innate immune system predominates at this stage with 70% of decidual leukocytes consisting of natural killer cells (NK), 20-25% macrophages and about 2% dendritic cells (Mor 2006). Approximately 1-3% of decidual immune cells at this time are adaptive system T lymphocytes; no B cells are present (Lessin et al., 1988). Thus, some of the immunoregulatory genes of interest could also be of maternal origin. Finally, one cannot exclude the potential contribution of circulating fetal or maternal immune cells in the placenta (Huppertz et al., 2007).

Surprisingly, a number of differentially expressed genes may be found in decidual stroma, including MUC15 (Shyu et al., 2007), IGFBP1 (Bischof et al., 1989), and PAEP (Jeschke et al., 2005). Although the goal of CVS is to obtain chorionic tissue for fetal genetic diagnosis, maternal decidual tissue is invariably present, as corroborated by our microarray analysis. Decidual tissue likely derives from placental septae projecting upwards from the basal plate towards the chorionic plate that contain an admixture of decidual cells, EVTs, and occasional trophoblast giant cells (Benirschke et al., 2006). On balance, the results suggest that preeclampsia may be associated with impaired decidualization. Whether this is etiological or secondary to suboptimal interaction with and stimulation by trophoblasts or maternal immune cells, or both is currently unknown. An alternative explanation, albeit less likely, is that there are fewer of these septae in early preeclampsia placentas, thus decreasing decidual tissue and consequently decidual gene expression in these CVS specimens.

Ten genes of interest were not found in any of the 72 Function and Disease categories assessed by IPA or located in current literature searches in conjunction with preeclampsia: CTAG2, MUC15, OXGR1, SCARA5, MAGEB6, TNRC9, TMC4, DEPDC7, RUFY3, and LAIR2. We suggest functional groupings, other than immune/inflammation, integrating these with all genes of interest (Table 4).

In order to examine hypotheses concerning hypoxia inducible transcription factors and oxidative stress, a secondary analysis of fold changes was conducted with the caveat that a high rate of false positives could be expected (Table 5) (Patel 2004). The concept that the placenta is hypoxic or over-expresses HIF-2α protein during early gestation, thereby impairing trophoblast invasion in preeclampsia, (Rajakumar et al., 2001) is not corroborated by this microarray analysis. We interrogated 26 genes proven to be HIF target genes (Wenger et al., 2005) and were been shown to be over-represented in placentas delivered from preeclamptic women (Tables 2 and 9) (Rajakumar et al., 2007). Only IGFBP1, WT1 and TH genes showed differential expression in one probe. Moreover, IGFBP1 and TH are typically up- and not down-regulated by hypoxia (Rajakumar et al., 2004; Tazuke et al., 1998). Interestingly, EPAS1 or HIF-2α (Tables 2 and 9) expression was markedly decreased, but did not consistently correlate with putative specific HIF-2α target genes (Table 3), (Lofstedt et al., 2007) suggesting adequate HIF-2α protein levels, transcriptional activity or compensation despite markedly reduced HIF-2α mRNA expression.

Nor were we able to support the differential expression of oxidative stress regulated genes at this early stage of pregnancy (Jauniaux et al., 2000; Many et al. 2000). We interrogated fold changes in 11 genes previously shown to be regulated by oxidative stress (Table 7), (Allen et al., 2000) and expression differences were nonsignificant. In fact, blood flow and oxygen delivery to the intervillous space begins around 10-12 weeks of gestation (Jauniaux et al., 2000), but expression profiles of the hypoxia (Table 6) and oxidative stress (Table 7) regulated genes do not support the concept of an undue delay or acceleration of this crucial physiological event, respectively. Thus, ischemia-hypoxia and oxidative stress due to reperfusion injury are likely to be later events in preeclampsia.

Noteworthy is that 17 of the 36 genes identified by the Naïve Bayes prediction model and J5 test were among the 152 identified by 2-fold FC analysis. Thus, there is considerable intersection of the two analytical approaches. The finding of aberrant decidualization in early placentas of preeclampsia revealed by the prediction modeling is bolstered by the FC analysis, insofar as FSTL3 (FC −2.56) (Jones et al., 2002) and prolactin (FC −7.86) (Telgmann 1998) are downregulated (Table 5). Additionally, marked downregulation of granulysin in the FC analysis (FC −23.51) further supports immune dysregulation in decidua (Mincheva-Nilsson et al., 2000).

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

TABLE 1A
Clinical characteristics of total microarray study sample (N = 12).

| | Control (N = 8) | Preeclampsia^{†} (N = 4) |
|---|---|---|
| Maternal age (years) | 38.1 ± 3.1 | 36.5 ± 0.6 |
| Nulliparous (N) | 7 | 4 |
| BMI at CVS | 24.5 ± 4.0 | 29.9 ± 4.2 |
| Gestational weeks at CVS | 11.3 ± 0.6 | 11.4 ± 0.7 |
| Gestational weeks at delivery | 39.8 ± 1.2 | 37.5 ± 2.9 |
| Birth weight (grams) | 3346.7 ± 437.4 | 2717.8 + 643.1 |
| Infant sex | | |
| Female | 4 | 2 |
| Male | 4 | 2 |
| Smoking | 1 | 0 |

^{†}Preeclampsia definition is based on the National High Blood Pressure Education Program Working Group (Gifford et al., 2000).
Mean ± SD; level of significance, p ≦ 0.05.

TABLE 1B
Additional data for preeclampsia cases.

| | | | | | | | | | Average | Average |
|---|---|---|---|---|---|---|---|---|---|---|
| PE case | Maternal | | | | | | | | systolic BP | diastolic BP |
| (Patient | age at | | | | | Gestational | Gestational | Mode of | (prior to 20 | (prior to 20 |
| ID) | CVS | Gravidity | Parity | Race | BMI | age at CVS | age at delivery | delivery | wks) | wks) |
| 1 (19) | 37 | 2 | 0 | W | 27.5 | 11.4 | 38 | C-section | 109 | 69 |
| 2 (21) | 36 | 2 | 0 | W | 36.0 | 12.4 | 35.7 | Vaginal | 116 | 69 |
| 3 (58) | 36 | 1 | 0 | W | 26.8 | 10.7 | 41.3 | Vaginal | 129 | 77 |
| 4 (147) | 37 | 1 | 0 | W | 29.1 | 11.0 | 34.9 | C-section | 102 | 69 |

TABLE 1C
Further additional data for preeclampsia cases.

| PE case | Average | Average | Proteinuria | Uric acid (>1SD | Birth | Birth | |
|---|---|---|---|---|---|---|---|
| (Patient | systolic BP | diastolic BP | (grams per 24 | for gestational age [+], | weight | weight | |
| ID) | (at delivery) | (at delivery) | hour urine) | mg/dL) | (grams) | percentile | Other |
| 1 (19) | 145 | 80 | 1.06 | 10.2 (+) | 3241 | >10 | Max BP 155/90, Low |
| | | | | | | | platelets to 87K, |
| | | | | | | | creatinine 1.1 mg/dL |
| 2 (21) | 155 | 90 | 4.76 | 8.1 (+) | 1965 | <3 | Max BP 164/88, |
| | | | | | | | IUGR |
| 3 (58) | 163 | 80 | 0.77 | 4.0 (−) | 3265 | >10 | Max systolic BP 184 |
| 4 (147) | 151 | 93 | 0.74 | 5.2 (+) | 2400 | >10 | Max BP 176/115 |

TABLE 2
Genes differentially expressed in first trimester PE.

| | | Condition in | | |
|---|---|---|---|---|
| | J5 Score | Preclampsia | Gene | |
| Probe Set ID | (3 vs 8) | Group | Symbol | Target Description |
| 1561318_at | −9.691 | Underexpressed | — | gb: BC043169.1 /DB_XREF = gi: 27693226 |
| | | | | /TID = Hs2.438491.1 /CNT = 2 /FEA = mRNA |
| | | | | /TIER = ConsEnd /STK = 1 /UG = Hs.438491 |
| | | | | /UG_TITLE = Homo sapiens, clone |
| | | | | IMAGE: 5287025, mRNA /DEF = Homo sapiens, |
| | | | | clone IMAGE: 5287025, mRNA. |
| 1562053_at | −8.578 | Underexpressed | — | gb: BC043556.1 /DB_XREF = gi: 27694238 |
| | | | | /TID = Hs2.432875.1 /CNT = 3 /FEA = mRNA |
| | | | | /TIER = ConsEnd /STK = 0 /UG = Hs.432875 |
| | | | | /UG_TITLE = Homo sapiens, clone |
| | | | | IMAGE: 5184785, mRNA /DEF = Homo sapiens, |
| | | | | clone IMAGE: 5184785, mRNA. |
| 234601_x_at | 8.373 | Overexpressed | — | gb: AK026385.1 /DB_XREF = gi: 10439235 |
| | | | | /FEA = mRNA /CNT = 1 /TID = Hs.306841.0 |
| | | | | /TIER = ConsEnd /STK = 0 /UG = Hs.306841 |
| | | | | /UG_TITLE = Homo sapiens cDNA: FLJ22732 fis, |
| | | | | clone HSI15880 /DEF = Homo sapiens cDNA: |
| | | | | FLJ22732 fis, clone HSI15880. |
| 242842_at | −14.512 | Underexpressed | — | gb: BF435734 /DB_XREF = gi: 11448036 |
| | | | | /DB_XREF = nab41a03.x1 |
| | | | | /CLONE = IMAGE: 3268133 /FEA = EST /CNT = 4 |
| | | | | /TID = Hs.301745.0 /TIER = ConsEnd /STK = 3 |
| | | | | /UG = Hs.301745 /UG_TITLE = ESTs, Weakly similar |
| | | | | to ALU1_HUMAN ALU SUBFAMILY J |
| | | | | SEQUENCE CONTAMINATION WARNING |
| | | | | ENTRY (H. sapiens) |
| 207607_at | −9.23 | Underexpressed | ASCL2 | gb: NM_005170.1 /DB_XREF = gi: 4885664 |
| | | | | /GEN = ASCL2 /FEA = FLmRNA /CNT = 3 |
| | | | | /TID = Hs.135639.0 /TIER = FL /STK = 0 |
| | | | | /UG = Hs.135639 /LL = 430 /DEF = Homo sapiens |
| | | | | achaete-scute complex (Drosophila) homolog-like 2 |
| | | | | (ASCL2), mRNA. /PROD = achaete-scute complex |
| | | | | homolog-like 2 /FL = gb: NM_005170.1 |
| 215141_at | 13.765 | Overexpressed | C4orf10 | gb: AA493300 /DB_XREF = gi: 2223141 |
| | | | | /DB_XREF = ng97e11.s1 /CLONE = IMAGE: 942764 |
| | | | | /FEA = mRNA /CNT = 6 /TID = Hs.104258.2 |
| | | | | /TIER = ConsEnd /STK = 0 /UG = Hs.104258 |
| | | | | /UG_TITLE = Homo sapiens mRNA, exon 1, 2, 3, 4, |
| | | | | clone: RES4-24A |
| 1568554_x_at | −10.67 | Underexpressed | C6orf142 | gb: AF317590.1 /DB_XREF = gi: 12751196 |
| | | | | /TID = Hs2.383544.1 /CNT = 1 /FEA = mRNA |
| | | | | /TIER = ConsEnd /STK = 0 /UG = Hs.383544 |
| | | | | /UG_TITLE = Homo sapiens T cell receptor beta |
| | | | | chain (BV16S1) mRNA, partial cds /DEF = Homo |
| | | | | sapiens T cell receptor beta chain (BV16S1) mRNA, |
| | | | | partial cds. |
| 205827_at | 20.414 | Overexpressed | CCK | gb: NM_000729.2 /DB_XREF = gi: 4755130 |
| | | | | /GEN = CCK /FEA = FLmRNA /CNT = 40 |
| | | | | /TID = Hs.80247.0 /TIER = FL + Stack /STK = 23 |
| | | | | /UG = Hs.80247 /LL = 885 /DEF = Homo sapiens |
| | | | | cholecystokinin (CCK), mRNA. |
| | | | | /PROD = cholecystokinin preproprotein |
| | | | | /FL = gb: NM_000729.2 |
| 215388_s_at | −8.449 | Underexpressed | CFH /// | gb: X56210.1 /DB_XREF = gi: 30132 /GEN = H 36-2 |
| | | | CFHR1 | /FEA = mRNA /CNT = 4 /TID = Hs.296941.0 |
| | | | | /TIER = ConsEnd /STK = 0 /UG = Hs.296941 /LL = 3079 |
| | | | | /DEF = H. sapiens mRNA for complement Factor H- |
| | | | | related protein 1, clone H 36-2. /PROD = FHR-1; |
| | | | | complement Factor H-related protein 1 |
| 215733_x_at | 8.519 | Overexpressed | CTAG2 | gb: AJ012833.1 /DB_XREF = gi: 3893216 |
| | | | | /GEN = CAMEL /FEA = mRNA /CNT = 5 |
| | | | | /TID = Hs.87225.1 /TIER = ConsEnd /STK = 0 |
| | | | | /UG = Hs.87225 /LL = 30848 /DEF = Homo sapiens |
| | | | | mRNA for CTL-recognized antigen on melanoma |
| | | | | (CAMEL). /PROD = CTL-recognized antigen on |
| | | | | melanoma (CAMEL) |
| 228293_at | −9.406 | Underexpressed | DEPDC7 | gb: AJ245600.1 /DB_XREF = gi: 5763726 |
| | | | | /GEN = TR2D15 /FEA = mRNA /CNT = 32 |
| | | | | /TID = Hs.180545.0 /TIER = Stack /STK = 18 |
| | | | | /UG = Hs.180545 /DEF = Homo sapiens mRNA for |
| | | | | hypothetical protein (TR2D15 gene). |
| | | | | /PROD = hypothetical protein |
| 235592_at | −8.249 | Underexpressed | ELL2 | gb: AW960145 /DB_XREF = gi: 8149829 |
| | | | | /DB_XREF = EST372216 /FEA = EST /CNT = 12 |
| | | | | /TID = Hs.100636.0 /TIER = ConsEnd /STK = 0 |
| | | | | /UG = Hs.100636 /UG_TITLE = ESTs |
| 242868_at | −15.342 | Underexpressed | EPAS1 | gb: T70087 /DB_XREF = gi: 681235 |
| | | | | /DB_XREF = yc17g11.s1 /CLONE = IMAGE: 80996 |
| | | | | /FEA = EST /CNT = 7 /TID = Hs.307559.0 |
| | | | | /TIER = ConsEnd /STK = 1 /UG = Hs.307559 |
| | | | | /UG_TITLE = ESTs |
| 226482_s_at | −8.441 | Underexpressed | F11R | gb: AI814545 /DB_XREF = gi: 5425760 |
| | | | | /DB_XREF = wj74d11.x1 |
| | | | | /CLONE = IMAGE: 2408565 /FEA = EST /CNT = 181 |
| | | | | /TID = Hs.12284.0 /TIER = Stack /STK = 23 |
| | | | | /UG = Hs.12284 /UG_TITLE = Homo sapiens, clone |
| | | | | IMAGE: 2989556, mRNA, partial cds |
| 214702_at | −8.2 | Underexpressed | FN1 | gb: AJ276395.1 /DB_XREF = gi: 12053816 /GEN = FN |
| | | | | /FEA = mRNA /CNT = 52 /TID = Hs.321592.0 |
| | | | | /TIER = ConsEnd /STK = 4 /UG = Hs.321592 |
| | | | | /DEF = Homo sapiens mRNA for MSF-FN70 (FN |
| | | | | gene). /PROD = migration stimulation factor FN70 |
| 203592_s_at | −10.048 | Underexpressed | FSTL3 | gb: NM_005860.1 /DB_XREF = gi: 5031700 |
| | | | | /GEN = FSTL3 /FEA = FLmRNA /CNT = 129 |
| | | | | /TID = Hs.25348.0 /TIER = FL + Stack /STK = 33 |
| | | | | /UG = Hs.25348 /LL = 10272 /DEF = Homo sapiens |
| | | | | follistatin-like 3 (secreted glycoprotein) (FSTL3), |
| | | | | mRNA. /PROD = follistatin-like 3 glycoprotein |
| | | | | /FL = gb: U76702.1 gb: NM_005860.1 |
| 241036_at | −9.764 | Underexpressed | HPS3 | gb: AA215451 /DB_XREF = gi: 1815195 |
| | | | | /DB_XREF = zr97d04.s1 /CLONE = IMAGE: 683623 |
| | | | | /FEA = EST /CNT = 4 /TID = Hs.189025.0 |
| | | | | /TIER = ConsEnd /STK = 4 /UG = Hs.189025 |
| | | | | /UG_TITLE = ESTs |
| 205302_at | −10.347 | Underexpressed | IGFBP1 | gb: NM_000596.1 /DB_XREF = gi: 4504614 |
| | | | | /GEN = IGFBP1 /FEA = FLmRNA /CNT = 268 |
| | | | | /TID = Hs.102122.0 /TIER = FL + Stack /STK = 117 |
| | | | | /UG = Hs.102122 /LL = 3484 /DEF = Homo sapiens |
| | | | | insulin-like growth factor binding protein 1 |
| | | | | (IGFBP1), mRNA. /PROD = insulin-like growth |
| | | | | factor binding protein 1 /FL = gb: NM_000596.1 |
| | | | | gb: M31145.1 gb: M20841.1 |
| 209351_at | −8.699 | Underexpressed | KRT14 | gb: BC002690.1 /DB_XREF = gi: 12803708 |
| | | | | /FEA = FLmRNA /CNT = 212 /TID = Hs.117729.0 |
| | | | | /TIER = FL + Stack /STK = 53 /UG = Hs.117729 |
| | | | | /LL = 3861 /UG_GENE = KRT14 /DEF = Homo |
| | | | | sapiens, keratin 14 (epidermolysis bullosa simplex, |
| | | | | Dowling-Meara, Koebner), clone MGC: 3944, |
| | | | | mRNA, complete cds. /PROD = keratin 14 |
| | | | | (epidermolysis bullosa simplex, Dowling-Meara, |
| | | | | Koebner) /FL = gb: NM_000526.1 gb: BC002690.1 |
| 207509_s_at | −18.871 | Underexpressed | LAIR2 | gb: NM_002288.2 /DB_XREF = gi: 10947100 |
| | | | | /GEN = LAIR2 /FEA = FLmRNA /CNT = 6 |
| | | | | /TID = Hs.43803.1 /TIER = FL /STK = 5 |
| | | | | /UG = Hs.43803 /LL = 3904 /DEF = Homo sapiens |
| | | | | leukocyte-associated Ig-like receptor 2 (LAIR2), |
| | | | | transcript variant 1, mRNA. /PROD = leukocyte- |
| | | | | associated Ig-like receptor 2, isoforma |
| | | | | /FL = gb: NM_002288.2 |
| 239010_at | −8.134 | Underexpressed | LOC440157 | gb: AI744280 /DB_XREF = gi: 5112568 |
| | | | | /DB_XREF = tr08f08.x1 /CLONE = IMAGE: 2217735 |
| | | | | /FEA = EST /CNT = 9 /TID = Hs.227056.0 |
| | | | | /TIER = ConsEnd /STK = 1 /UG = Hs.227056 |
| | | | | /UG_TITLE = ESTs, Moderately similar to |
| | | | | mitochondrial outer membrane protein (H. sapiens) |
| 219759_at | −9.916 | Underexpressed | LRAP | gb: NM_022350.1 /DB_XREF = gi: 11641260 |
| | | | | /GEN = LOC64167 /FEA = FLmRNA /CNT = 18 |
| | | | | /TID = Hs.280380.0 /TIER = FL /STK = 0 |
| | | | | /UG = Hs.280380 /LL = 64167 /DEF = Homo sapiens |
| | | | | aminopeptidase (LOC64167), mRNA. |
| | | | | /PROD = aminopeptidase /FL = gb: AF191545.1 |
| | | | | gb: NM_022350.1 |
| 1552858_at | −8.915 | Underexpressed | MAGEB6 | gb: NM_173523.1 /DB_XREF = gi: 27734966 |
| | | | | /TID = Hs2.376011.1 /CNT = 4 /FEA = FLmRNA |
| | | | | /TIER = FL /STK = 2 /LL = 158809 |
| | | | | /UG_GENE = FLJ40242 /UG = Hs.376011 |
| | | | | /UG_TITLE = hypothetical protein FLJ40242 |
| | | | | /DEF = Homo sapiens hypothetical protein FLJ40242 |
| | | | | (FLJ40242), mRNA. /FL = gb: NM_173523.1 |
| 204580_at | −17.183 | Underexpressed | MMP12 | gb: NM_002426.1 /DB_XREF = gi: 4505206 |
| | | | | /GEN = MMP12 /FEA = FLmRNA /CNT = 72 |
| | | | | /TID = Hs.1695.0 /TIER = FL + Stack /STK = 18 |
| | | | | /UG = Hs.1695 /LL = 4321 /DEF = Homo sapiens |
| | | | | matrix metalloproteinase 12 (macrophage elastase) |
| | | | | (MMP12), mRNA. /PROD = matrix metalloproteinase |
| | | | | 12 preproprotein /FL = gb: L23808.1 |
| | | | | gb: NM_002426.1 |
| 227238_at | −8.014 | Underexpressed | MUC15 | gb: W93847 /DB_XREF = gi: 1422970 |
| | | | | /DB_XREF = zd97a07.s1 /CLONE = IMAGE: 357396 |
| | | | | /FEA = mRNA /CNT = 89 /TID = Hs.24139.0 |
| | | | | /TIER = Stack /STK = 21 /UG = Hs.24139 |
| | | | | /UG_TITLE = Homo sapiens cDNA: FLJ23137 fis, |
| | | | | clone LNG08842 |
| 1553319_at | −8.245 | Underexpressed | OXGR1 | gb: AF370886.1 /DB_XREF = gi: 21728283 |
| | | | | /GEN = GPR99 /TID = Hs2.352218.1 /CNT = 4 |
| | | | | /FEA = FLmRNA /TIER = FL /STK = 1 /LL = 27199 |
| | | | | /UG = Hs.352218 /DEF = Homo sapiens G protein- |
| | | | | coupled receptor GPR99 (GPR99) mRNA, complete |
| | | | | cds. /PROD = G protein-coupled receptor GPR99 |
| | | | | /FL = gb: AF370886.1 gb: NM_080818.1 |
| 206859_s_at | −15.642 | Underexpressed | PAEP | gb: NM_002571.1 /DB_XREF = gi: 4505582 |
| | | | | /GEN = PAEP /FEA = FLmRNA /CNT = 18 |
| | | | | /TID = Hs.82269.0 /TIER = FL + Stack /STK = 13 |
| | | | | /UG = Hs.82269 /LL = 5047 /DEF = Homo sapiens |
| | | | | progestagen-associated endometrial protein |
| | | | | (placental protein 14, pregnancy-associated |
| | | | | endometrial alpha-2-globulin, alpha uterine protein) |
| | | | | (PAEP), mRNA. /PROD = progestagen-associated |
| | | | | endometrial protein(placental protein 14, pregnancy- |
| | | | | associated endometrialalpha-2-globulin, alpha |
| | | | | uterine protein) /FL = gb: NM_002571.1 gb: J04129.1 |
| 210251_s_at | −9.553 | Underexpressed | RUFY3 | gb: AF112221.1 /DB_XREF = gi: 6563227 |
| | | | | /FEA = FLmRNA /CNT = 13 /TID = Hs.7972.1 |
| | | | | /TIER = FL /STK = 5 /UG = Hs.7972 /LL = 22902 |
| | | | | /UG_GENE = KIAA0871 /DEF = Homo sapiens rap2 |
| | | | | interacting protein x mRNA, complete cds. |
| | | | | /PROD = rap2 interacting protein x |
| | | | | /FL = gb: AF112221.1 |
| 202917_s_at | 13.185 | Overexpressed | S100A8 | gb: NM_002964.2 /DB_XREF = gi: 9845519 |
| | | | | /GEN = S100A8 /FEA = FLmRNA /CNT = 257 |
| | | | | /TID = Hs.100000.0 /TIER = FL + Stack /STK = 93 |
| | | | | /UG = Hs.100000 /LL = 6279 /DEF = Homo sapiens |
| | | | | S100 calcium-binding protein A8 (calgranulin A) |
| | | | | (S100A8), mRNA. /PROD = S100 calcium-binding |
| | | | | protein A8 /FL = gb: NM_002964.2 |
| 1554276_at | −13.803 | Underexpressed | SART3 | gb: BC041638.1 /DB_XREF = gi: 27371321 |
| | | | | /TID = Hs2.116875.2 /CNT = 7 /FEA = FLmRNA |
| | | | | /TIER = FL /STK = 2 /LL = 9733 /UG_GENE = SART3 |
| | | | | /UG = Hs.116875 /DEF = Homo sapiens, Similar to |
| | | | | squamous cell carcinoma antigen recognised by T |
| | | | | cells 3, clone MGC: 52127 IMAGE: 4844061, |
| | | | | mRNA, complete cds. /PROD = Similar to squamous |
| | | | | cell carcinoma antigenrecognised by T cells 3 |
| | | | | /FL = gb: BC041638.1 |
| 229839_at | −8.3 | Underexpressed | SCARA5 | gb: AI799784 /DB_XREF = gi: 5365256 |
| | | | | /DB_XREF = wc43b08.x1 |
| | | | | /CLONE = IMAGE: 2321367 /FEA = EST /CNT = 10 |
| | | | | /TID = Hs.49696.0 /TIER = Stack /STK = 9 |
| | | | | /UG = Hs.49696 /UG_TITLE = ESTs |
| 203789_s_at | −8.419 | Underexpressed | SEMA3C | gb: NM_006379.1 /DB_XREF = gi: 5454047 |
| | | | | /GEN = SEMA3C /FEA = FLmRNA /CNT = 92 |
| | | | | /TID = Hs.171921.0 /TIER = FL + Stack /STK = 18 |
| | | | | /UG = Hs.171921 /LL = 10512 /DEF = Homo sapiens |
| | | | | sema domain, immunoglobulin domain (Ig), short |
| | | | | basic domain, secreted, (semaphorin) 3C (SEMA3C), |
| | | | | mRNA. /PROD = sema domain, immunoglobulin |
| | | | | domain (Ig), shortbasic domain, secreted, |
| | | | | (semaphorin) 3C /FL = gb: NM_006379.1 |
| | | | | gb: AB000220.1 |
| 230748_at | −8.374 | Underexpressed | SLC16A6 | gb: AI873273 /DB_XREF = gi: 5547322 |
| | | | | /DB_XREF = wf41c12.x1 |
| | | | | /CLONE = IMAGE: 2358166 /FEA = EST /CNT = 20 |
| | | | | /TID = Hs.42645.0 /TIER = Stack /STK = 10 |
| | | | | /UG = Hs.42645 /UG_TITLE = ESTs |
| 219911_s_at | −8.586 | Underexpressed | SLCO4A1 | gb: NM_016354.1 /DB_XREF = gi: 7706516 |
| | | | | /GEN = SLC21A12 /FEA = FLmRNA /CNT = 19 |
| | | | | /TID = Hs.235782.0 /TIER = FL /STK = 1 |
| | | | | /UG = Hs.235782 /LL = 28231 /DEF = Homo sapiens |
| | | | | solute carrier family 21 (organic anion transporter), |
| | | | | member 12 (SLC21A12), mRNA. /PROD = organic |
| | | | | anion transporter OATP-E /FL = gb: AB031051.1 |
| | | | | gb: NM_016354.1 gb: AF205072.1 gb: AF187817.1 |
| 226403_at | −8.969 | Underexpressed | TMC4 | gb: BE645551 /DB_XREF = gi: 9969862 |
| | | | | /DB_XREF = 7e72b08.x1 |
| | | | | /CLONE = IMAGE: 3287991 /FEA = EST /CNT = 44 |
| | | | | /TID = Hs.112184.1 /TIER = Stack /STK = 27 |
| | | | | /UG = Hs.112184 /LL = 26173 |
| | | | | /UG_GENE = DKFZP586J0619 |
| | | | | /UG_TITLE = DKFZP586J0619 protein |
| 215108_x_at | −8.953 | Underexpressed | TNRC9 | gb: U80736.1 /DB_XREF = gi: 2565047 |
| | | | | /GEN = CAGF9 /FEA = mRNA /CNT = 8 |
| | | | | /TID = Hs.110826.1 /TIER = ConsEnd /STK = 0 |
| | | | | /UG = Hs.110826 /LL = 27324 /DEF = Homo sapiens |
| | | | | CAGF9 mRNA, partial cds. /PROD = CAGF9 |

TABLE 3
Top Functions and Diseases in IPA associated with high priority genes of interest.

| Function and Disease | −log(p- | |
|---|---|---|
| Category | value) | Genes* |
| Cellular Movement | 1.31E−06-2.89E−02 | F11R, FN1, S100A8, PAEP, SEMA3C, IGFBP1, |
| | | CCK, MMP12 |
| Hematological | 1.31E−06-2.82E−02 | F11R, FN1, S100A8, IGFBP1, PAEP, MMP12 |
| System Development | | |
| and Function | | |
| Cancer | 2.5E−06-2.89E−02 | KRT14, LRAP, FN1, EPAS1, FSTL3, CCK, PAEP, |
| | | HPS3, F11R, S100A8, IGFBP1, SEMA3C, MMP12, |
| | | CFHR1 |
| Immune Response | 4.11E−06-2.82E−02 | LRAP, F11R, FN1, S100A8, IGFBP1, PAEP, |
| | | SEMA3C, |
| | | MMP12, CFHR1 |
| Reproductive System | 4.81E−06-2.75E−02 | HPS3, EPAS1, FN1, FSTL3, S100A8, SEMA3C, |
| Disease | | PAEP, |
| | | IGFBP1, ELL2 |
| Hematological | 3.83E−05-2.91E−02 | F11R, FN1, EPAS1, IGFBP1 |
| Disease | | |
| Gastrointestinal | 6.36E−04-3.05E−02 | KRT14, LRAP, FN1, FSTL3, CCK, IGFBP1, CFHR1 |
| Disease | | |
| Nutritional Disease | 7.52E−04-2.89E−02 | FN1, IGFBP1, CCK |
| Reproductive System | 8.14E−04-2.26E−02 | EPAS1, FN1, IGFBP1 |
| Development | | |
| and Function | | |
| Embryonic | 1.56E−03-2.26E−02 | EPAS1, FN1, ASCL2, SEMA3C |
| Development | | |

Table created in IPA for genes of interest, Dec. 23, 2007 (Ingenuity 2007);
*All genes are down-regulated except CCK and S100A8.

TABLE 4
Functional groups other than immune incorporating
IPA-omitted high priority genes of interest in PE.

| Functional group | Genes of Interest |
|---|---|
| Neural/Axonal | CCK (Hansen et al., 2001; Hansen |
| | et al., 2007; Sodowski et al., 2007) |
| | SEMA3C (Baun et al., 2006; |
| | Gonthier et al., 2007) |
| | RUFY3 (Mori et al., 2007) |
| | ASCL2 (Westerman et al., 2002) |
| | DEPDC7 (Wong et al., 2000) |
| Imprinting | CTAG2 (Lethe et al., 1998; Graves |
| | 1998) |
| | ASCL (Miyamoto et al., 2002; |
| | Westerman et al., 2001) |
| Angiogenesis | SEMA3C (Feiner et al., 2001) |
| | EPAS1 (Lelievre et al., 2001) |
| | PAEP (Song et al., 2001) |
| | CCK (Chao et al., 2007) |
| | FN1 (Kim et al., 2000) |
| VEGF related | PAEP (Song et al., 2001) |
| | KRT14 (Kiba et al., 2003) |
| | FR11 (Kang et al., 2007) |
| | CCK (Chao et al., 2007) |
| Epithelium | S100A8 (Wilkinson et al., 1988) |
| | PAEP (Jeschke et al., 2005) |
| | F11R (Table 9) |
| | MUC15 (Shyu et al., 2007) |
| | SCARA5 (Jiang et al., 2006) |
| | SCLO4A1 (Gao et al., 2005) |
| | KRT14 (Ma et al., 2001) |
| | LAIR2 (Xu et al., 2005) |
| Melanoma | CTAG2 (Vaughan et al., 2004; |
| | Jungbluth et al., 2007) |
| | MAGEB (Jungbluth et al., 2007) |
| | LAIR2 (Xu et al., 2005) |
| Endothelium | CCK HUVEC (Lefranc et al., |
| | 2004) |
| | PAEP HUVEC (Song et al., 2001) |
| | LAIR2 (Xu et al., 2005) |
| | CFH (Manuelian et al., 2003) |
| | S100A8 (Sato et al., 1999) |
| | SEMA3C (Banu et al., 2006) |
| | EPAS1 (Sato et al., 2004) |
| Transcription factor | ASCL2 |
| | EPAS1 |
| | TNRC9 |
| | ELL2 |
| | (all in Table 9) |
| Metabolic homeostasis | CCK (Sodowski et al., 2007) |
| Insulin/glucose metabolism | FSTL3 (Mukherjee et al., 2007) |
| | IGFBP1 (Table 9) |
| | EPAS1 (Wada 2007) |
| | SLCO4A1 (Fujiwara et al., 2005) |
| | SLC16A6 (Table 9) |
| Tyrosine kinase | SEMA3C (Table 9) |
| | RUFY3 (Yang et al., 2002) |
| | FN1 (Table 9) |
| G protein-coupled receptor | OXGR1 (Table 9) |
| | DEPDC7 (Wong et al., 2000) |

TABLE 5
Fold change data for CVS microarray

| | | | Condition in | |
|---|---|---|---|---|
| | Fold | | Preclampsia | |
| Probe ID | Change | Gene Symbols | Group | Target Description |
| 236087_at | 2.22 | ABLIM2 | Overexpressed | gb: AI912773 |
| | | | | /DB_XREF = gi: 5632628 |
| | | | | /DB_XREF = we13e06.x1 |
| | | | | /CLONE = IMAGE: 2340994 |
| | | | | /FEA = EST /CNT = 9 |
| | | | | /TID = Hs.62648.0 |
| | | | | /TIER = ConsEnd /STK = 2 |
| | | | | /UG = Hs.62648 |
| | | | | /UG_TITLE = ESTs |
| 214358_at | −2.12 | ACACA | Underexpressed | gb: AW188201 |
| | | | | /DB_XREF = gi: 6462637 |
| | | | | /DB_XREF = xj93f06.x1 |
| | | | | /CLONE = IMAGE: 2664803 |
| | | | | /FEA = EST /CNT = 8 |
| | | | | /TID = Hs.7232.1 /TIER = Stack |
| | | | | /STK = 8 /UG = Hs.7232 |
| | | | | /LL = 31 /UG_GENE = ACACA |
| | | | | /UG_TITLE = acetyl- |
| | | | | Coenzyme A carboxylase |
| | | | | alpha |
| 236514_at | 2.09 | ACOT8 | Overexpressed | gb: AI885067 |
| | | | | /DB_XREF = gi: 5590231 |
| | | | | /DB_XREF = wl89c05.x1 |
| | | | | /CLONE = IMAGE: 2432072 |
| | | | | /FEA = EST /CNT = 7 |
| | | | | /TID = Hs.6511.0 |
| | | | | /TIER = ConsEnd /STK = 6 |
| | | | | /UG = Hs.6511 |
| | | | | /UG_TITLE = ESTs |
| 210929_s_at | 2.39 | AHSG | Overexpressed | gb: AF130057.1 |
| | | | | /DB_XREF = gi: 11493420 |
| | | | | /FEA = FLmRNA /CNT = 2 |
| | | | | /TID = Hs.323288.0 /TIER = FL |
| | | | | /STK = 0 /UG = Hs.323288 |
| | | | | /DEF = Homo sapiens clone |
| | | | | FLB5539 PRO1454 mRNA, |
| | | | | complete cds. |
| | | | | /PROD = PRO1454 |
| | | | | /FL = gb: AF130057.1 |
| 207016_s_at | −7.83 | ALDH1A2 | Underexpressed | gb: AB015228.1 |
| | | | | /DB_XREF = gi: 3970845 |
| | | | | /GEN = RALDH2 |
| | | | | /FEA = FLmRNA /CNT = 10 |
| | | | | /TID = Hs.95197.0 /TIER = FL |
| | | | | /STK = 0 /UG = Hs.95197 |
| | | | | /LL = 8854 /DEF = Homo |
| | | | | sapiens mRNA for RALDH2- |
| | | | | T, complete cds. |
| | | | | /PROD = RALDH2-T |
| | | | | /FL = gb: AB015228.1 |
| | | | | gb: AB015227.1 |
| | | | | gb: AB015226.1 |
| | | | | gb: NM_003888.1 |
| 243276_at | −2.19 | ALS2CL | Underexpressed | gb: AA557247 |
| | | | | /DB_XREF = gi: 2327724 |
| | | | | /DB_XREF = nl75e08.s1 |
| | | | | /CLONE = IMAGE: 1056518 |
| | | | | /FEA = EST /CNT = 6 |
| | | | | /TID = Hs.293637.1 |
| | | | | /TIER = ConsEnd /STK = 0 |
| | | | | /UG = Hs.293637 |
| | | | | /UG_TITLE = ESTs |
| 208323_s_at | 2.89 | ANXA13 | Overexpressed | gb: NM_004306.1 |
| | | | | /DB_XREF = gi: 4757753 |
| | | | | /GEN = ANXA13 |
| | | | | /FEA = FLmRNA /CNT = 2 |
| | | | | /TID = Hs.181107.0 /TIER = FL |
| | | | | /STK = 0 /UG = Hs.181107 |
| | | | | /LL = 312 /DEF = Homo sapiens |
| | | | | annexin A13 (ANXA13), |
| | | | | mRNA. /PROD = annexin A13 |
| | | | | /FL = gb: NM_004306.1 |
| 203527_s_at | −2.58 | APC | Underexpressed | gb: NM_000038.1 |
| | | | | /DB_XREF = gi: 4557318 |
| | | | | /GEN = APC /FEA = FLmRNA |
| | | | | /CNT = 154 /TID = Hs.75081.0 |
| | | | | /TIER = FL /STK = 0 |
| | | | | /UG = Hs.75081 /LL = 324 |
| | | | | /DEF = Homo sapiens |
| | | | | adenomatosis polyposis coli |
| | | | | (APC), mRNA. |
| | | | | /PROD = adenomatosis |
| | | | | polyposis coli |
| | | | | /FL = gb: M74088.1 |
| | | | | gb: M73548.1 |
| | | | | gb: NM_000038.1 |
| 206672_at | −2.04 | AQP2 | Underexpressed | gb: NM_000486.2 |
| | | | | /DB_XREF = gi: 4755122 |
| | | | | /GEN = AQP2 |
| | | | | /FEA = FLmRNA /CNT = 26 |
| | | | | /TID = Hs.37025.0 |
| | | | | /TIER = FL + Stack /STK = 17 |
| | | | | /UG = Hs.37025 /LL = 359 |
| | | | | /DEF = Homo sapiens |
| | | | | aquaporin 2 (collecting duct) |
| | | | | (AQP2), mRNA. |
| | | | | /PROD = aquaporin 2 |
| | | | | /FL = gb: NM_000486.2 |
| 239373_at | 3.41 | ARFGEF2 | Overexpressed | gb: AI034357 |
| | | | | /DB_XREF = gi: 3255310 |
| | | | | /DB_XREF = ox20e11.x1 |
| | | | | /CLONE = IMAGE: 1656908 |
| | | | | /FEA = EST /CNT = 7 |
| | | | | /TID = Hs.211194.0 |
| | | | | /TIER = ConsEnd /STK = 1 |
| | | | | /UG = Hs.211194 |
| | | | | /UG_TITLE = ESTs, Weakly |
| | | | | similar to ALU8_HUMAN |
| | | | | ALU SUBFAMILY SX |
| | | | | SEQUENCE |
| | | | | CONTAMINATION |
| | | | | WARNING ENTRY |
| | | | | (H. sapiens) |
| 207919_at | −2.2 | ART1 | Underexpressed | gb: NM_004314.1 |
| | | | | /DB_XREF = gi: 4757783 |
| | | | | /GEN = ART1 |
| | | | | /FEA = FLmRNA /CNT = 2 |
| | | | | /TID = Hs.73139.0 /TIER = FL |
| | | | | /STK = 0 /UG = Hs.73139 |
| | | | | /LL = 417 /DEF = Homo sapiens |
| | | | | ADP-ribosyltransferase 1 |
| | | | | (ART1), mRNA. |
| | | | | /PROD = ADP- |
| | | | | ribosyltransferase 1 |
| | | | | /FL = gb: NM_004314.1 |
| 229215_at | −2.15 | ASCL2 | Underexpressed | gb: AI393930 |
| | | | | /DB_XREF = gi: 4223477 |
| | | | | /DB_XREF = tg11a01.x1 |
| | | | | /CLONE = IMAGE: 2108424 |
| | | | | /FEA = EST /CNT = 18 |
| | | | | /TID = Hs.152475.0 |
| | | | | /TIER = Stack /STK = 8 |
| | | | | /UG = Hs.152475 |
| | | | | /UG_TITLE = ESTs |
| 233796_at | 2.23 | ATP7B | Overexpressed | gb: AK002066.1 |
| | | | | /DB_XREF = gi: 7023723 |
| | | | | /FEA = mRNA /CNT = 2 |
| | | | | /TID = Hs.296547.0 |
| | | | | /TIER = ConsEnd /STK = 0 |
| | | | | /UG = Hs.296547 |
| | | | | /UG_TITLE = Homo sapiens |
| | | | | cDNA FLJ11204 fis, clone |
| | | | | PLACE1007810 /DEF = Homo |
| | | | | sapiens cDNA FLJ11204 fis, |
| | | | | clone PLACE1007810. |
| 212849_at | −2.17 | AXIN1 | Underexpressed | gb: AA745954 |
| | | | | /DB_XREF = gi: 2785940 |
| | | | | /DB_XREF = ob18e04.s1 |
| | | | | /CLONE = IMAGE: 1324062 |
| | | | | /FEA = mRNA /CNT = 72 |
| | | | | /TID = Hs.184434.0 |
| | | | | /TIER = Stack /STK = 33 |
| | | | | /UG = Hs.184434 /LL = 8312 |
| | | | | /UG_GENE = AXIN1 |
| | | | | /UG_TITLE = axin |
| 205870_at | −2.7 | BDKRB2 | Underexpressed | gb: NM_000623.1 |
| | | | | /DB_XREF = gi: 4557358 |
| | | | | /GEN = BDKRB2 |
| | | | | /FEA = FLmRNA /CNT = 34 |
| | | | | /TID = Hs.250882.0 |
| | | | | /TIER = FL + Stack /STK = 17 |
| | | | | /UG = Hs.250882 /LL = 624 |
| | | | | /DEF = Homo sapiens |
| | | | | bradykinin receptor B2 |
| | | | | (BDKRB2), mRNA. |
| | | | | /PROD = bradykinin receptor |
| | | | | B2 /FL = gb: M88714.1 |
| | | | | gb: NM_000623.1 |
| 204741_at | 2.19 | BICD1 | Overexpressed | gb: NM_001714.1 |
| | | | | /DB_XREF = gi: 4502408 |
| | | | | /GEN = BICD1 |
| | | | | /FEA = FLmRNA /CNT = 47 |
| | | | | /TID = Hs.164975.0 |
| | | | | /TIER = FL + Stack /STK = 10 |
| | | | | /UG = Hs.164975 /LL = 636 |
| | | | | /DEF = Homo sapiens Bicaudal |
| | | | | D (Drosophila) homolog 1 |
| | | | | (BICD1), mRNA. |
| | | | | /PROD = Bicaudal D |
| | | | | (Drosophila) homolog 1 |
| | | | | /FL = gb: U90028.1 |
| | | | | gb: NM_001714.1 |
| 217767_at | −3.28 | C3 | Underexpressed | gb: NM_000064.1 |
| | | | | /DB_XREF = gi: 4557384 |
| | | | | /GEN = C3 /FEA = FLmRNA |
| | | | | /CNT = 473 /TID = Hs.284394.0 |
| | | | | /TIER = FL + Stack /STK = 213 |
| | | | | /UG = Hs.284394 /LL = 718 |
| | | | | /DEF = Homo sapiens |
| | | | | complement component 3 |
| | | | | (C3), mRNA. |
| | | | | /PROD = complement |
| | | | | component 3 precursor |
| | | | | /FL = gb: K02765.1 |
| | | | | gb: NM_000064.1 |
| 205654_at | −2.67 | C4BPA | Underexpressed | gb: NM_000715.1 |
| | | | | /DB_XREF = gi: 4502502 |
| | | | | /GEN = C4BPA |
| | | | | /FEA = FLmRNA /CNT = 53 |
| | | | | /TID = Hs.1012.0 |
| | | | | /TIER = FL + Stack /STK = 22 |
| | | | | /UG = Hs.1012 /LL = 722 |
| | | | | /DEF = Homo sapiens |
| | | | | complement component 4- |
| | | | | binding protein, alpha |
| | | | | (C4BPA), mRNA. |
| | | | | /PROD = complement |
| | | | | component 4-binding protein, |
| | | | | alpha /FL = gb: NM_000715.1 |
| | | | | gb: M31452.1 |
| 205827_at | 4.46 | CCK | Overexpressed | gb: NM_000729.2 |
| | | | | /DB_XREF = gi: 4755130 |
| | | | | /GEN = CCK /FEA = FLmRNA |
| | | | | /CNT = 40 /TID = Hs.80247.0 |
| | | | | /TIER = FL + Stack /STK = 23 |
| | | | | /UG = Hs.80247 /LL = 885 |
| | | | | /DEF = Homo sapiens |
| | | | | cholecystokinin (CCK), |
| | | | | mRNA. |
| | | | | /PROD = cholecystokinin |
| | | | | preproprotein |
| | | | | /FL = gb: NM_000729.2 |
| 34210_at | 2.42 | CD52 | Overexpressed | Cluster Incl. |
| | | | | N90866: zb11b10.s1 Homo |
| | | | | sapiens cDNA, 3 end |
| | | | | /clone = IMAGE-301723 |
| | | | | /clone_end = 3′ /gb = N90866 |
| | | | | /gi = 1444193 /ug = Hs.214742 |
| | | | | /len = 577 |
| 206328_at | 7.67 | CDH15 | Overexpressed | gb: NM_004933.1 |
| | | | | /DB_XREF = gi: 4826668 |
| | | | | /GEN = CDH15 |
| | | | | /FEA = FLmRNA /CNT = 32 |
| | | | | /TID = Hs.148090.0 /TIER = FL |
| | | | | /STK = 6 /UG = Hs.148090 |
| | | | | /LL = 1013 /DEF = Homo |
| | | | | sapiens cadherin 15, M- |
| | | | | cadherin (myotubule) |
| | | | | (CDH15), mRNA. |
| | | | | /PROD = cadherin 15, M- |
| | | | | cadherin (myotubule) |
| | | | | /FL = gb: D83542.1 |
| | | | | gb: NM_004933.1 |
| 239026_x_at | −2.68 | CENTG3 | Underexpressed | gb: H20019 |
| | | | | /DB_XREF = gi: 888714 |
| | | | | /DB_XREF = yn55c04.s1 |
| | | | | /CLONE = IMAGE: 172326 |
| | | | | /FEA = EST /CNT = 8 |
| | | | | /TID = Hs.148415.0 |
| | | | | /TIER = ConsEnd /STK = 1 |
| | | | | /UG = Hs.148415 |
| | | | | /UG_TITLE = ESTs |
| 215388_s_at | −3.48 | CFHR1 | Underexpressed | gb: X56210.1 |
| | | | | /DB_XREF = gi: 30132 |
| | | | | /GEN = H 36-2 /FEA = mRNA |
| | | | | /CNT = 4 /TID = Hs.296941.0 |
| | | | | /TIER = ConsEnd /STK = 0 |
| | | | | /UG = Hs.296941 /LL = 3079 |
| | | | | /DEF = H. sapiens mRNA for |
| | | | | complement Factor H-related |
| | | | | protein 1, clone H 36-2. |
| | | | | /PROD = FHR-1; complement |
| | | | | Factor H-related protein 1 |
| 1568704_a_at | −3.76 | CHERP | Underexpressed | gb: BF511683 |
| | | | | /DB_XREF = gi: 11594981 |
| | | | | /DB_XREF = UI-H-BI4-aom- |
| | | | | c-07-0-UI.s1 |
| | | | | /CLONE = IMAGE: 3085428 |
| | | | | /TID = Hs2.300452.1 /CNT = 9 |
| | | | | /FEA = mRNA |
| | | | | /TIER = ConsEnd /STK = 2 |
| | | | | /UG = Hs.300452 |
| | | | | /UG_TITLE = Homo sapiens, |
| | | | | Similar to calcium |
| | | | | homeostasis endoplasmic |
| | | | | reticulum protein, clone |
| | | | | IMAGE: 4827033, mRNA |
| | | | | (Probes match on alternative |
| | | | | forms of same gene) |
| 209763_at | −7.34 | CHRDL1 | Underexpressed | gb: AL049176 |
| | | | | /DB_XREF = gi: 4808226 |
| | | | | /FEA = FLmRNA /CNT = 84 |
| | | | | /TID = Hs.82223.0 |
| | | | | /TIER = Stack /STK = 46 |
| | | | | /UG = Hs.82223 /LL = 57803 |
| | | | | /UG_GENE = LOC57803 |
| | | | | /UG_TITLE = chordin-like |
| | | | | /DEF = Human DNA sequence |
| | | | | from clone 141H5 on |
| | | | | chromosome Xq22.1-23. |
| | | | | Contains parts of a novel |
| | | | | Chordin LIKE protein with |
| | | | | von Willebrand factor type C |
| | | | | domains. Contains ESTs, |
| | | | | STSs and GSSs |
| | | | | /FL = gb: BC002909.1 |
| 223786_at | −2.21 | CHST6 | Underexpressed | gb: AF280086.1 |
| | | | | /DB_XREF = gi: 12060803 |
| | | | | /GEN = GST4beta |
| | | | | /FEA = FLmRNA /CNT = 9 |
| | | | | /TID = Hs.157439.1 /TIER = FL |
| | | | | /STK = 0 /UG = Hs.157439 |
| | | | | /LL = 4166 /DEF = Homo |
| | | | | sapiens N-acetylglucosamine |
| | | | | 6-O-sulfotransferase GST- |
| | | | | 4beta mRNA, complete cds. |
| | | | | /PROD = N-acetylglucosamine |
| | | | | 6-O-sulfotransferaseGST- |
| | | | | 4beta /FL = gb: AF280086.1 |
| 1555469_a_at | −2.1 | CLASP2 | Underexpressed | gb: BC029035.1 |
| | | | | /DB_XREF = gi: 20810064 |
| | | | | /TID = Hs2.108614.1 /CNT = 1 |
| | | | | /FEA = FLmRNA /TIER = FL |
| | | | | /STK = 1 /LL = 23122 |
| | | | | /UG_GENE = CLASP2 |
| | | | | /UG = Hs.108614 /DEF = Homo |
| | | | | sapiens, Similar to CLIP- |
| | | | | associating protein 2, clone |
| | | | | MGC: 35497 |
| | | | | IMAGE: 5197008, mRNA, |
| | | | | complete cds. /PROD = Similar |
| | | | | to CLIP-associating protein 2 |
| | | | | /FL = gb: BC029035.1 (Probes |
| | | | | match on alternative forms of |
| | | | | same gene) |
| 209235_at | −2.13 | CLCN7 | Underexpressed | gb: AL031600 |
| | | | | /DB_XREF = gi: 4826481 |
| | | | | /FEA = FLmRNA /CNT = 184 |
| | | | | /TID = Hs.80768.0 |
| | | | | /TIER = Stack /STK = 75 |
| | | | | /UG = Hs.80768 /LL = 1186 |
| | | | | /UG_GENE = CLCN7 |
| | | | | /UG_TITLE = chloride channel |
| | | | | 7 /DEF = Human DNA |
| | | | | sequence from clone 390E6 |
| | | | | on chromosome 16. Contains |
| | | | | the 3 part of the CLCN7 gene |
| | | | | for chloride channel 7, ESTs, |
| | | | | a GSS and four putative CpG |
| | | | | islands /FL = gb: AF224741.1 |
| 237810_at | −2.15 | CLDN6 | Underexpressed | gb: AW003929 |
| | | | | /DB_XREF = gi: 5850845 |
| | | | | /DB_XREF = wq84d05.x1 |
| | | | | /CLONE = IMAGE: 2478729 |
| | | | | /FEA = EST /CNT = 6 |
| | | | | /TID = Hs.198684.0 |
| | | | | /TIER = ConsEnd /STK = 6 |
| | | | | /UG = Hs.198684 |
| | | | | /UG_TITLE = ESTs |
| 213818_x_at | 2.12 | COL5A1 | Overexpressed | gb: AI862325 |
| | | | | /DB_XREF = gi: 5526432 |
| | | | | /DB_XREF = tw71h04.x1 |
| | | | | /CLONE = IMAGE: 2265175 |
| | | | | /FEA = EST /CNT = 23 |
| | | | | /TID = Hs.146428.3 |
| | | | | /TIER = Stack /STK = 19 |
| | | | | /UG = Hs.146428 /LL = 1289 |
| | | | | /UG_GENE = COL5A1 |
| | | | | /UG_TITLE = collagen, type |
| | | | | V, alpha 1 |
| 204724_s_at | 2.41 | COL9A3 | Overexpressed | gb: NM_001853.1 |
| | | | | /DB_XREF = gi: 4502966 |
| | | | | /GEN = COL9A3 |
| | | | | /FEA = FLmRNA /CNT = 331 |
| | | | | /TID = Hs.53563.0 |
| | | | | /TIER = FL + Stack /STK = 14 |
| | | | | /UG = Hs.53563 /LL = 1299 |
| | | | | /DEF = Homo sapiens collagen, |
| | | | | type IX, alpha 3 (COL9A3), |
| | | | | mRNA. /PROD = collagen, |
| | | | | type IX, alpha 3 |
| | | | | /FL = gb: L41162.1 |
| | | | | gb: NM_001853.1 |
| 235019_at | −2.06 | CPM | Underexpressed | gb: BE878495 |
| | | | | /DB_XREF = gi: 10327271 |
| | | | | /DB_XREF = 601492515F1 |
| | | | | /CLONE = IMAGE: 3894722 |
| | | | | /FEA = EST /CNT = 32 |
| | | | | /TID = Hs.267158.0 |
| | | | | /TIER = ConsEnd /STK = 0 |
| | | | | /UG = Hs.267158 |
| | | | | /UG_TITLE = ESTs |
| 217552_x_at | 2 | CR1 | Overexpressed | gb: AI432713 |
| | | | | /DB_XREF = gi: 4283899 |
| | | | | /DB_XREF = th43e02.x1 |
| | | | | /CLONE = IMAGE: 2121050 |
| | | | | /FEA = EST /CNT = 5 |
| | | | | /TID = Hs.241053.0 |
| | | | | /TIER = ConsEnd /STK = 4 |
| | | | | /UG = Hs.241053 |
| | | | | /UG_TITLE = ESTs |
| 206185_at | −2.05 | CRYBB1 | Underexpressed | gb: NM_001887.1 |
| | | | | /DB_XREF = gi: 4503060 |
| | | | | /GEN = CRYBB1 |
| | | | | /FEA = FLmRNA /CNT = 41 |
| | | | | /TID = Hs.37135.0 /TIER = FL |
| | | | | /STK = 7 /UG = Hs.37135 |
| | | | | /LL = 1414 /DEF = Homo |
| | | | | sapiens crystallin, beta B1 |
| | | | | (CRYBB1), mRNA. |
| | | | | /PROD = crystallin, beta B1 |
| | | | | /FL = gb: U35340.1 |
| | | | | gb: NM_001887.1 |
| 243286_at | −2.01 | CUL1 | Underexpressed | gb: AA682674 |
| | | | | /DB_XREF = gi: 2669955 |
| | | | | /DB_XREF = zj20h10.s1 |
| | | | | /CLONE = IMAGE: 450883 |
| | | | | /FEA = EST /CNT = 6 |
| | | | | /TID = Hs.140498.0 |
| | | | | /TIER = ConsEnd /STK = 2 |
| | | | | /UG = Hs.140498 |
| | | | | /UG_TITLE = ESTs |
| 1554837_a_at | −2.38 | CYP4A11(includes | Underexpressed | gb: BC041158.1 |
| | | EG: 1579) | | /DB_XREF = gi: 27552844 |
| | | | | /TID = Hs2.1645.2 /CNT = 3 |
| | | | | /FEA = FLmRNA /TIER = FL |
| | | | | /STK = 1 /LL = 1579 |
| | | | | /UG_GENE = CYP4A11 |
| | | | | /UG = Hs.1645 /DEF = Homo |
| | | | | sapiens, Similar to |
| | | | | cytochrome P450, subfamily |
| | | | | IVA, polypeptide 11, clone |
| | | | | MGC: 48730 |
| | | | | IMAGE: 5183289, mRNA, |
| | | | | complete cds. /PROD = Similar |
| | | | | to cytochrome P450, |
| | | | | subfamily IVA, polypeptide 11 |
| | | | | /FL = gb: BC041158.1 (Probes |
| | | | | match on alternative forms of |
| | | | | same gene) |
| 203915_at | 2.07 | CXCL9 | Overexpressed | gb: NM_002416.1 |
| | | | | /DB_XREF = gi: 4505186 |
| | | | | /GEN = MIG /FEA = FLmRNA |
| | | | | /CNT = 103 /TID = Hs.77367.0 |
| | | | | /TIER = FL + Stack /STK = 38 |
| | | | | /UG = Hs.77367 /LL = 4283 |
| | | | | /DEF = Homo sapiens |
| | | | | monokine induced by gamma |
| | | | | interferon (MIG), mRNA. |
| | | | | /PROD = monokine induced by |
| | | | | gamma interferon |
| | | | | /FL = gb: NM_002416.1 |
| 214079_at | −2.77 | DHRS2 (includes | Underexpressed | gb: AK000345.1 |
| | | EG: 10202) | | /DB_XREF = gi: 7020368 |
| | | | | /FEA = mRNA /CNT = 16 |
| | | | | /TID = Hs.152677.0 |
| | | | | /TIER = Stack /STK = 12 |
| | | | | /UG = Hs.152677 |
| | | | | /UG_TITLE = Homo sapiens |
| | | | | cDNA FLJ20338 fis, clone |
| | | | | HEP12179 /DEF = Homo |
| | | | | sapiens cDNA FLJ20338 fis, |
| | | | | clone HEP12179. |
| 1568736_s_at | −4.42 | DLGAP1 | Underexpressed | gb: BC030096.1 |
| | | | | /DB_XREF = gi: 22535265 |
| | | | | /TID = Hs2.371203.1 /CNT = 5 |
| | | | | /FEA = mRNA |
| | | | | /TIER = ConsEnd /STK = 2 |
| | | | | /UG = Hs.371203 |
| | | | | /UG_TITLE = Homo sapiens, |
| | | | | clone IMAGE: 4795078, |
| | | | | mRNA /DEF = Homo sapiens, |
| | | | | clone IMAGE: 4795078, |
| | | | | mRNA. (All probes match |
| | | | | over all sequences) |
| 205493_s_at | 2.01 | DPYSL4 | Overexpressed | gb: NM_006426.1 |
| | | | | /DB_XREF = gi: 11321616 |
| | | | | /GEN = DPYSL4 |
| | | | | /FEA = FLmRNA /CNT = 29 |
| | | | | /TID = Hs.100058.0 /TIER = FL |
| | | | | /STK = 1 /UG = Hs.100058 |
| | | | | /LL = 10570 /DEF = Homo |
| | | | | sapiens dihydropyrimidinase- |
| | | | | like 4 (DPYSL4), mRNA. |
| | | | | /PROD = dihydropyrimidinase- |
| | | | | like 4 /FL = gb: NM_006426.1 |
| | | | | gb: AB006713.1 |
| 222847_s_at | −2.06 | EGLN3 | Underexpressed | gb: AI378406 |
| | | | | /DB_XREF = gi: 4188259 |
| | | | | /DB_XREF = tc78g03.x1 |
| | | | | /CLONE = IMAGE: 2070772 |
| | | | | /FEA = FLmRNA /CNT = 47 |
| | | | | /TID = Hs.18878.0 |
| | | | | /TIER = Stack /STK = 17 |
| | | | | /UG = Hs.18878 /LL = 63900 |
| | | | | /UG_GENE = FLJ21620 |
| | | | | /UG_TITLE = hypothetical |
| | | | | protein FLJ21620 |
| | | | | /FL = gb: NM_022073.1 |
| 227404_s_at | 2.37 | EGR1 | Overexpressed | gb: AI459194 |
| | | | | /DB_XREF = gi: 4311773 |
| | | | | /DB_XREF = tj54g06.x1 |
| | | | | /CLONE = IMAGE: 2145370 |
| | | | | /FEA = EST /CNT = 42 |
| | | | | /TID = Hs.326035.1 |
| | | | | /TIER = Stack /STK = 26 |
| | | | | /UG = Hs.326035 /LL = 1958 |
| | | | | /UG_GENE = EGR1 |
| | | | | /UG_TITLE = early growth |
| | | | | response 1 |
| 235592_at | −4.02 | ELL2 | Underexpressed | gb: AW960145 |
| | | | | /DB_XREF = gi: 8149829 |
| | | | | /DB_XREF = EST372216 |
| | | | | /FEA = EST /CNT = 12 |
| | | | | /TID = Hs.100636.0 |
| | | | | /TIER = ConsEnd /STK = 0 |
| | | | | /UG = Hs.100636 |
| | | | | /UG_TITLE = ESTs |
| 242868_at | −7.64 | EPAS1 | Underexpressed | gb: T70087 |
| | | | | /DB_XREF = gi: 681235 |
| | | | | /DB_XREF = yc17g11.s1 |
| | | | | /CLONE = IMAGE: 80996 |
| | | | | /FEA = EST /CNT = 7 |
| | | | | /TID = Hs.307559.0 |
| | | | | /TIER = ConsEnd /STK = 1 |
| | | | | /UG = Hs.307559 |
| | | | | /UG_TITLE = ESTs |
| 203989_x_at | −2.98 | F2R | Underexpressed | gb: NM_001992.2 |
| | | | | /DB_XREF = gi: 6031164 |
| | | | | /GEN = F2R /FEA = FLmRNA |
| | | | | /CNT = 74 /TID = Hs.128087.0 |
| | | | | /TIER = FL + Stack /STK = 10 |
| | | | | /UG = Hs.128087 /LL = 2149 |
| | | | | /DEF = Homo sapiens |
| | | | | coagulation factor II |
| | | | | (thrombin) receptor (F2R), |
| | | | | mRNA. /PROD = coagulation |
| | | | | factor II receptor precursor |
| | | | | /FL = gb: M62424.1 |
| | | | | gb: BC002464.1 |
| | | | | gb: NM_001992.2 |
| 239248_at | 2.1 | FKBP1A | Overexpressed | gb: BE742802 |
| | | | | /DB_XREF = gi: 10156794 |
| | | | | /DB_XREF = 601574420F1 |
| | | | | /CLONE = IMAGE: 3835445 |
| | | | | /FEA = EST /CNT = 11 |
| | | | | /TID = Hs.189696.0 |
| | | | | /TIER = ConsEnd /STK = 0 |
| | | | | /UG = Hs.189696 |
| | | | | /UG_TITLE = ESTs |
| 204358_s_at | 2.2 | FLRT2 | Overexpressed | gb: AF169676.1 |
| | | | | /DB_XREF = gi: 6808604 |
| | | | | /GEN = FLRT2 |
| | | | | /FEA = FLmRNA /CNT = 86 |
| | | | | /TID = Hs.48998.0 |
| | | | | /TIER = FL + Stack /STK = 10 |
| | | | | /UG = Hs.48998 /LL = 23768 |
| | | | | /DEF = Homo sapiens leucine- |
| | | | | rich repeat transmembrane |
| | | | | protein FLRT2 (FLRT2) |
| | | | | mRNA, complete cds. |
| | | | | /PROD = leucine-rich repeat |
| | | | | transmembrane protein |
| | | | | FLRT2 /FL = gb: AB007865.1 |
| | | | | gb: AF169676.1 |
| | | | | gb: NM_013231.1 |
| 229902_at | −2.12 | FLT4 | Underexpressed | gb: AW083785 |
| | | | | /DB_XREF = gi: 6038937 |
| | | | | /DB_XREF = xc35b04.x1 |
| | | | | /CLONE = IMAGE: 2586223 |
| | | | | /FEA = EST /CNT = 14 |
| | | | | /TID = Hs.8941.0 /TIER = Stack |
| | | | | /STK = 11 /UG = Hs.8941 |
| | | | | /UG_TITLE = ESTs |
| 214702_at | −2.35 | FN1 | Underexpressed | gb: AJ276395.1 |
| | | | | /DB_XREF = gi: 12053816 |
| | | | | /GEN = FN /FEA = mRNA |
| | | | | /CNT = 52 /TID = Hs.321592.0 |
| | | | | /TIER = ConsEnd /STK = 4 |
| | | | | /UG = Hs.321592 /DEF = Homo |
| | | | | sapiens mRNA for MSF- |
| | | | | FN70 (FN gene). |
| | | | | /PROD = migration stimulation |
| | | | | factor FN70 |
| 209189_at | 2.78 | FOS | Overexpressed | gb: BC004490.1 |
| | | | | /DB_XREF = gi: 13325363 |
| | | | | /FEA = FLmRNA /CNT = 139 |
| | | | | /TID = Hs.25647.0 /TIER = FL |
| | | | | /STK = 0 /UG = Hs.25647 |
| | | | | /LL = 2353 /UG_GENE = FOS |
| | | | | /DEF = Homo sapiens, v-fos |
| | | | | FBJ murine osteosarcoma |
| | | | | viral oncogene homolog, |
| | | | | clone MGC: 11074, mRNA, |
| | | | | complete cds. /PROD = v-fos |
| | | | | FBJ murine osteosarcoma |
| | | | | viral oncogenehomolog |
| | | | | /FL = gb: NM_005252.2 |
| | | | | gb: BC004490.1 |
| 202768_at | 2.33 | FOSB | Overexpressed | gb: NM_006732.1 |
| | | | | /DB_XREF = gi: 5803016 |
| | | | | /GEN = FOSB |
| | | | | /FEA = FLmRNA /CNT = 167 |
| | | | | /TID = Hs.75678.0 |
| | | | | /TIER = FL + Stack /STK = 67 |
| | | | | /UG = Hs.75678 /LL = 2354 |
| | | | | /DEF = Homo sapiens FBJ |
| | | | | murine osteosarcoma viral |
| | | | | oncogene homolog B (FOSB), |
| | | | | mRNA. /PROD = FBJ murine |
| | | | | osteosarcoma viral oncogene |
| | | | | homologB |
| | | | | /FL = gb: NM_006732.1 |
| | | | | gb: L49169.1 |
| 214560_at | 2 | FPRL2 | Overexpressed | gb: NM_002030.2 |
| | | | | /DB_XREF = gi: 4758401 |
| | | | | /GEN = FPRL2 |
| | | | | /FEA = FLmRNA /CNT = 4 |
| | | | | /TID = Hs.158314.0 /TIER = FL |
| | | | | /STK = 0 /UG = Hs.158314 |
| | | | | /LL = 2359 /DEF = Homo |
| | | | | sapiens formyl peptide |
| | | | | receptor-like 2 (FPRL2), |
| | | | | mRNA. /PROD = formyl |
| | | | | peptide receptor-like 2 |
| | | | | /FL = gb: NM_002030.2 |
| | | | | gb: M76673.1 |
| 203592_s_at | −2.56 | FSTL3 | Underexpressed | gb: NM_005860.1 |
| | | | | /DB_XREF = gi: 5031700 |
| | | | | /GEN = FSTL3 |
| | | | | /FEA = FLmRNA /CNT = 129 |
| | | | | /TID = Hs.25348.0 |
| | | | | /TIER = FL + Stack /STK = 33 |
| | | | | /UG = Hs.25348 /LL = 10272 |
| | | | | /DEF = Homo sapiens |
| | | | | follistatin-like 3 (secreted |
| | | | | glycoprotein) (FSTL3), |
| | | | | mRNA. /PROD = follistatin- |
| | | | | like 3 glycoprotein |
| | | | | /FL = gb: U76702.1 |
| | | | | gb: NM_005860.1 |
| 1565661_x_at | 2.02 | FUT6 | Overexpressed | gb: BC040472.1 |
| | | | | /DB_XREF = gi: 26251793 |
| | | | | /TID = Hs2.434404.1 /CNT = 6 |
| | | | | /FEA = mRNA |
| | | | | /TIER = ConsEnd /STK = 0 |
| | | | | /UG = Hs.434404 |
| | | | | /UG_TITLE = Homo sapiens, |
| | | | | clone IMAGE: 4557161, |
| | | | | mRNA /DEF = Homo sapiens, |
| | | | | clone IMAGE: 4557161, |
| | | | | mRNA (Probes cross hyb with |
| | | | | other sequences) |
| 206136_at | 2.24 | FZD5 | Overexpressed | gb: NM_003468.1 |
| | | | | /DB_XREF = gi: 4503828 |
| | | | | /GEN = FZD5 /FEA = FLmRNA |
| | | | | /CNT = 19 /TID = Hs.152251.0 |
| | | | | /TIER = FL /STK = 0 |
| | | | | /UG = Hs.152251 /LL = 7855 |
| | | | | /DEF = Homo sapiens frizzled |
| | | | | (Drosophila) homolog 5 |
| | | | | (FZD5), mRNA. |
| | | | | /PROD = frizzled 5 |
| | | | | /FL = gb: NM_003468.1 |
| | | | | gb: U43318.1 |
| 1555590_a_at | −2.47 | GATA1 | Underexpressed | gb: BC009797.1 |
| | | | | /DB_XREF = gi: 14602570 |
| | | | | /TID = Hs2.765.2 /CNT = 1 |
| | | | | /FEA = FLmRNA /TIER = FL |
| | | | | /STK = 1 /LL = 2623 |
| | | | | /UG_GENE = GATA1 |
| | | | | /UG = Hs.765 /DEF = Homo |
| | | | | sapiens, GATA-binding |
| | | | | protein 1 (globin transcription |
| | | | | factor 1), clone MGC: 13628 |
| | | | | IMAGE: 4048082, mRNA, |
| | | | | complete cds. |
| | | | | /PROD = GATA-binding |
| | | | | protein 1 (globin |
| | | | | transcriptionfactor 1) |
| | | | | /FL = gb: BC009797.1 (Probes |
| | | | | match on alternative forms of |
| | | | | same gene) |
| 1555765_a_at | −3.73 | GNG4 | Underexpressed | gb: AF493872.1 |
| | | | | /DB_XREF = gi: 20147636 |
| | | | | /GEN = GNG4 |
| | | | | /TID = Hs2.32976.3 /CNT = 1 |
| | | | | /FEA = FLmRNA /TIER = FL |
| | | | | /STK = 1 /LL = 2786 |
| | | | | /UG = Hs.32976 /DEF = Homo |
| | | | | sapiens guanine nucleotide |
| | | | | binding protein gamma 4 |
| | | | | (GNG4) mRNA, complete |
| | | | | cds. /PROD = guanine |
| | | | | nucleotide binding protein |
| | | | | gamma 4 /FL = gb: AF493872.1 |
| | | | | (Probes match on alternative |
| | | | | forms of same gene) |
| 232043_at | −2.67 | GNG7 | Underexpressed | gb: AA890272 |
| | | | | /DB_XREF = gi: 3017151 |
| | | | | /DB_XREF = aj96e12.s1 |
| | | | | /CLONE = IMAGE: 1404334 |
| | | | | /FEA = mRNA /CNT = 14 |
| | | | | /TID = Hs.6750.1 |
| | | | | /TIER = ConsEnd /STK = 2 |
| | | | | /UG = Hs.6750 |
| | | | | /UG_TITLE = Homo sapiens |
| | | | | mRNA for FLJ00058 protein, |
| | | | | partial cds |
| 205495_s_at | −23.51 | GNLY | Underexpressed | gb: NM_006433.2 |
| | | | | /DB_XREF = gi: 7108343 |
| | | | | /GEN = GNLY |
| | | | | /FEA = FLmRNA /CNT = 32 |
| | | | | /TID = Hs.105806.1 |
| | | | | /TIER = FL + Stack /STK = 18 |
| | | | | /UG = Hs.105806 /LL = 10578 |
| | | | | /DEF = Homo sapiens |
| | | | | granulysin (GNLY), transcript |
| | | | | variant NKG5, mRNA. |
| | | | | /PROD = granulysin, isoform |
| | | | | NKG5 /FL = gb: NM_006433.2 |
| 205220_at | −2.63 | GPR109B | Underexpressed | gb: NM_006018.1 |
| | | | | /DB_XREF = gi: 5174460 |
| | | | | /GEN = HM74 |
| | | | | /FEA = FLmRNA /CNT = 40 |
| | | | | /TID = Hs.137555.0 |
| | | | | /TIER = FL + Stack /STK = 17 |
| | | | | /UG = Hs.137555 /LL = 8843 |
| | | | | /DEF = Homo sapiens putative |
| | | | | chemokine receptor; GTP- |
| | | | | binding protein (HM74), |
| | | | | mRNA. /PROD = putative |
| | | | | chemokine receptor; GTP- |
| | | | | bindingprotein |
| | | | | /FL = gb: NM_006018.1 |
| 223789_s_at | −2.17 | GTPBP2 | Underexpressed | gb: AF116627.1 |
| | | | | /DB_XREF = gi: 7959755 |
| | | | | /FEA = FLmRNA /CNT = 8 |
| | | | | /TID = Hs.183698.1 /TIER = FL |
| | | | | /STK = 0 /UG = Hs.183698 |
| | | | | /LL = 6159 |
| | | | | /UG_GENE = RPL29 |
| | | | | /DEF = Homo sapiens |
| | | | | PRO1181 mRNA, complete |
| | | | | cds. /PROD = PRO1181 |
| | | | | /FL = gb: AF116627.1 |
| 210164_at | −2.7 | GZMB | Underexpressed | gb: J03189.1 |
| | | | | /DB_XREF = gi: 338010 |
| | | | | /FEA = FLmRNA /CNT = 28 |
| | | | | /TID = Hs.1051.1 |
| | | | | /TIER = FL + Stack /STK = 11 |
| | | | | /UG = Hs.1051 /LL = 3002 |
| | | | | /UG_GENE = GZMB |
| | | | | /UG_TITLE = granzyme B |
| | | | | (granzyme 2, cytotoxic T- |
| | | | | lymphocyte-associated serine |
| | | | | esterase 1) /DEF = Human |
| | | | | proteolytic serine esterase-like |
| | | | | protein (SECT) gene, |
| | | | | complete cds. |
| | | | | /FL = gb: M17016.1 |
| | | | | gb: J03189.1 gb: NM_004131.2 |
| | | | | gb: J04071.1 |
| 205919_at | −2.64 | HBE1 | Underexpressed | gb: NM_005330.2 |
| | | | | /DB_XREF = gi: 6715605 |
| | | | | /GEN = HBE1 |
| | | | | /FEA = FLmRNA /CNT = 48 |
| | | | | /TID = Hs.117848.0 |
| | | | | /TIER = FL + Stack /STK = 10 |
| | | | | /UG = Hs.117848 /LL = 3046 |
| | | | | /DEF = Homo sapiens |
| | | | | hemoglobin, epsilon 1 |
| | | | | (HBE1), mRNA. |
| | | | | /PROD = hemoglobin, epsilon |
| | | | | 1 /FL = gb: NM_005330.2 |
| 206647_at | −6.51 | HBZ | Underexpressed | gb: NM_005332.2 |
| | | | | /DB_XREF = gi: 6633805 |
| | | | | /GEN = HBZ /FEA = FLmRNA |
| | | | | /CNT = 36 /TID = Hs.272003.0 |
| | | | | /TIER = FL + Stack /STK = 17 |
| | | | | /UG = Hs.272003 /LL = 3050 |
| | | | | /DEF = Homo sapiens |
| | | | | hemoglobin, zeta (HBZ), |
| | | | | mRNA. /PROD = hemoglobin, |
| | | | | zeta /FL = gb: NM_005332.2 |
| | | | | gb: M24173.1 |
| 202473_x_at | 2.17 | HCFC1 | Overexpressed | gb: AA703045 |
| | | | | /DB_XREF = gi: 2706158 |
| | | | | /DB_XREF = zi74d09.s1 |
| | | | | /CLONE = IMAGE: 436529 |
| | | | | /FEA = FLmRNA /CNT = 143 |
| | | | | /TID = Hs.83634.0 |
| | | | | /TIER = Stack /STK = 12 |
| | | | | /UG = Hs.83634 /LL = 3054 |
| | | | | /UG_GENE = HCFC1 |
| | | | | /UG_TITLE = host cell factor |
| | | | | C1 (VP16-accessory protein) |
| | | | | /FL = gb: NM_005334.1 |
| 1556426_at | 2.46 | HEXA | Overexpressed | gb: BC034424.1 |
| | | | | /DB_XREF = gi: 21706987 |
| | | | | /TID = Hs2.166299.1 /CNT = 8 |
| | | | | /FEA = mRNA |
| | | | | /TIER = ConsEnd /STK = 3 |
| | | | | /UG = Hs.166299 |
| | | | | /UG_TITLE = Homo sapiens, |
| | | | | clone IMAGE: 4823589, |
| | | | | mRNA, partial cds |
| | | | | /DEF = Homo sapiens, clone |
| | | | | IMAGE: 4823589, mRNA, |
| | | | | partial cds. |
| 203290_at | 2.21 | HLA-DQA1 | Overexpressed | gb: NM_002122.1 |
| | | | | /DB_XREF = gi: 4504406 |
| | | | | /GEN = HLA-DQA1 |
| | | | | /FEA = FLmRNA /CNT = 126 |
| | | | | /TID = Hs.198253.0 |
| | | | | /TIER = FL + Stack /STK = 37 |
| | | | | /UG = Hs.198253 /LL = 3117 |
| | | | | /DEF = Homo sapiens major |
| | | | | histocompatibility complex, |
| | | | | class II, DQ alpha 1 (HLA- |
| | | | | DQA1), mRNA. |
| | | | | /PROD = major |
| | | | | histocompatibility complex, |
| | | | | class II, DQalpha 1 |
| | | | | /FL = gb: NM_002122.1 |
| | | | | gb: M33906.1 gb: M16995.1 |
| | | | | gb: M26041.1 gb: M17847.1 |
| | | | | gb: M17846.1 |
| 204778_x_at | −2.24 | HOXB7 | Underexpressed | gb: AW102783 |
| | | | | /DB_XREF = gi: 6073396 |
| | | | | /DB_XREF = xd38a03.x1 |
| | | | | /CLONE = IMAGE: 2596012 |
| | | | | /FEA = FLmRNA /CNT = 52 |
| | | | | /TID = Hs.819.0 /TIER = Stack |
| | | | | /STK = 8 /UG = Hs.819 |
| | | | | /LL = 3217 |
| | | | | /UG_GENE = HOXB7 |
| | | | | /UG_TITLE = homeo box B7 |
| | | | | /FL = gb: NM_004502.1 |
| | | | | gb: M16937.1 |
| 241036_at | −3.01 | HPS3 | Underexpressed | gb: AA215451 |
| | | | | /DB_XREF = gi: 1815195 |
| | | | | /DB_XREF = zr97d04.s1 |
| | | | | /CLONE = IMAGE: 683623 |
| | | | | /FEA = EST /CNT = 4 |
| | | | | /TID = Hs.189025.0 |
| | | | | /TIER = ConsEnd /STK = 4 |
| | | | | /UG = Hs.189025 |
| | | | | /UG_TITLE = ESTs |
| 206638_at | −2.51 | HTR2B | Underexpressed | gb: NM_000867.1 |
| | | | | /DB_XREF = gi: 4504538 |
| | | | | /GEN = HTR2B |
| | | | | /FEA = FLmRNA /CNT = 13 |
| | | | | /TID = Hs.2507.0 |
| | | | | /TIER = FL + Stack /STK = 10 |
| | | | | /UG = Hs.2507 /LL = 3357 |
| | | | | /DEF = Homo sapiens 5- |
| | | | | hydroxytryptamine (serotonin) |
| | | | | receptor 2B (HTR2B), |
| | | | | mRNA. /PROD = 5- |
| | | | | hydroxytryptamine (serotonin) |
| | | | | receptor 2B |
| | | | | /FL = gb: NM_000867.1 |
| 1566502_at | −2.66 | HYDIN | Underexpressed | gb: AL157445.1 |
| | | | | /DB_XREF = gi: 7018447 |
| | | | | /TID = Hs2.375670.1 /CNT = 1 |
| | | | | /FEA = mRNA |
| | | | | /TIER = ConsEnd /STK = 0 |
| | | | | /UG = Hs.375670 |
| | | | | /UG TITLE = Homo sapiens |
| | | | | mRNA; cDNA |
| | | | | DKFZp434B049 (from clone |
| | | | | DKFZp434B049) |
| | | | | /DEF = Homo sapiens mRNA; |
| | | | | cDNA DKFZp434B049 (from |
| | | | | clone DKFZp434B049). |
| 205302_at | −53.23 | IGFBP1 | Underexpressed | gb: NM_000596.1 |
| | | | | /DB_XREF = gi: 4504614 |
| | | | | /GEN = IGFBP1 |
| | | | | /FEA = FLmRNA /CNT = 268 |
| | | | | /TID = Hs.102122.0 |
| | | | | /TIER = FL + Stack /STK = 117 |
| | | | | /UG = Hs.102122 /LL = 3484 |
| | | | | /DEF = Homo sapiens insulin- |
| | | | | like growth factor binding |
| | | | | protein 1 (IGFBP1), mRNA. |
| | | | | /PROD = insulin-like growth |
| | | | | factor binding protein 1 |
| | | | | /FL = gb: NM_000596.1 |
| | | | | gb: M31145.1 gb: M20841.1 |
| 221651_x_at | −2.38 | IGKC | Underexpressed | gb: BC005332.1 |
| | | | | /DB_XREF = gi: 13529115 |
| | | | | /FEA = FLmRNA /CNT = 8 |
| | | | | /TID = Hs.156110.0 /TIER = FL |
| | | | | /STK = 0 /UG = Hs.156110 |
| | | | | /LL = 3514 /UG_GENE = IGKC |
| | | | | /DEF = Homo sapiens, Similar |
| | | | | to immunoglobulin kappa |
| | | | | constant, clone MGC: 12418, |
| | | | | mRNA, complete cds. |
| | | | | /PROD = Similar to |
| | | | | immunoglobulin kappa |
| | | | | constant /FL = gb: AF113887.1 |
| | | | | gb: BC005332.1 |
| 205038_at | 2.06 | IKZF1 | Overexpressed | gb: BG540504 |
| | | | | /DB_XREF = gi: 13532737 |
| | | | | /DB_XREF = 602569230F1 |
| | | | | /CLONE = IMAGE: 4693783 |
| | | | | /FEA = FLmRNA /CNT = 54 |
| | | | | /TID = Hs.54452.0 |
| | | | | /TIER = ConsEnd /STK = 0 |
| | | | | /UG = Hs.54452 /LL = 10320 |
| | | | | /UG_GENE = ZNFN1A1 |
| | | | | /UG_TITLE = zinc finger |
| | | | | protein, subfamily 1A, 1 |
| | | | | (Ikaros) /FL = gb: U40462.1 |
| | | | | gb: NM_006060.1 |
| 205992_s_at | −2.76 | IL15 | Underexpressed | gb: NM_000585.1 |
| | | | | /DB_XREF = gi: 10835152 |
| | | | | /GEN = IL15 /FEA = FLmRNA |
| | | | | /CNT = 33 /TID = Hs.168132.0 |
| | | | | /TIER = FL /STK = 0 |
| | | | | /UG = Hs.168132 /LL = 3600 |
| | | | | /DEF = Homo sapiens |
| | | | | interleukin 15 (IL15), mRNA. |
| | | | | /PROD = interleukin 15 |
| | | | | /FL = gb: NM_000585.1 |
| | | | | gb: U14407.1 |
| 205067_at | −2.14 | IL1B | Underexpressed | gb: NM_000576.1 |
| | | | | /DB_XREF = gi: 10835144 |
| | | | | /GEN = IL1B /FEA = FLmRNA |
| | | | | /CNT = 97 /TID = Hs.126256.0 |
| | | | | /TIER = FL + Stack /STK = 15 |
| | | | | /UG = Hs.126256 /LL = 3553 |
| | | | | /DEF = Homo sapiens |
| | | | | interleukin 1, beta (IL1B), |
| | | | | mRNA. /PROD = interleukin 1, |
| | | | | beta /FL = gb: M15330.1 |
| | | | | gb: M54933.1 gb: K02770.1 |
| | | | | gb: NM_000576.1 |
| 207526_s_at | −2.93 | IL1RL1 | Underexpressed | gb: NM_003856.1 |
| | | | | /DB_XREF = gi: 4507244 |
| | | | | /GEN = IL1RL1 |
| | | | | /FEA = FLmRNA /CNT = 4 |
| | | | | /TID = Hs.66.0 /TIER = FL |
| | | | | /STK = 0 /UG = Hs.66 |
| | | | | /LL = 9173 /DEF = Homo |
| | | | | sapiens interleukin 1 receptor- |
| | | | | like 1 (IL1RL1), mRNA. |
| | | | | /PROD = interleukin 1 |
| | | | | receptor-like 1 |
| | | | | /FL = gb: NM_003856.1 |
| 205291_at | −2.77 | IL2RB | Underexpressed | gb: NM_000878.1 |
| | | | | /DB_XREF = gi: 4504664 |
| | | | | /GEN = IL2RB |
| | | | | /FEA = FLmRNA /CNT = 45 |
| | | | | /TID = Hs.75596.0 |
| | | | | /TIER = FL + Stack /STK = 26 |
| | | | | /UG = Hs.75596 /LL = 3560 |
| | | | | /DEF = Homo sapiens |
| | | | | interleukin 2 receptor, beta |
| | | | | (IL2RB), mRNA. |
| | | | | /PROD = interleukin 2 |
| | | | | receptor, beta |
| | | | | /FL = gb: NM_000878.1 |
| | | | | gb: M26062.1 |
| 204698_at | 2.74 | ISG20 | Overexpressed | gb: NM_002201.2 |
| | | | | /DB_XREF = gi: 6857799 |
| | | | | /GEN = ISG20 |
| | | | | /FEA = FLmRNA /CNT = 51 |
| | | | | /TID = Hs.183487.0 |
| | | | | /TIER = FL + Stack /STK = 21 |
| | | | | /UG = Hs.183487 /LL = 3669 |
| | | | | /DEF = Homo sapiens |
| | | | | interferon stimulated gene |
| | | | | (20 kD) (ISG20), mRNA. |
| | | | | /PROD = interferon stimulated |
| | | | | gene (20 kD) |
| | | | | /FL = gb: U88964.1 |
| | | | | gb: NM_002201.2 |
| 208084_at | −2.13 | ITGB6 | Underexpressed | gb: NM_000888.3 |
| | | | | /DB_XREF = gi: 9966771 |
| | | | | /GEN = ITGB6 |
| | | | | /FEA = FLmRNA /CNT = 2 |
| | | | | /TID = Hs.123125.0 /TIER = FL |
| | | | | /STK = 0 /UG = Hs.123125 |
| | | | | /LL = 3694 /DEF = Homo |
| | | | | sapiens integrin, beta 6 |
| | | | | (ITGB6), mRNA. |
| | | | | /PROD = integrin, beta 6 |
| | | | | /FL = gb: NM_000888.3 |
| | | | | gb: M35198.3 |
| 210036_s_at | −2.06 | KCNH2 | Underexpressed | gb: AB044806.1 |
| | | | | /DB_XREF = gi: 11933151 |
| | | | | /GEN = HERG |
| | | | | /FEA = FLmRNA /CNT = 34 |
| | | | | /TID = Hs.188021.1 |
| | | | | /TIER = FL + Stack /STK = 24 |
| | | | | /UG = Hs.188021 /LL = 3757 |
| | | | | /DEF = Homo sapiens HERG |
| | | | | mRNA for HERG-USO, |
| | | | | alternatively spliced, complete |
| | | | | cds. /PROD = HERG-USO |
| | | | | /FL = gb: AB044806.1 |
| 231774_at | −2.42 | KCNIP3 | Underexpressed | gb: R17174 |
| | | | | /DB_XREF = gi: 770784 |
| | | | | /DB_XREF = yg11a08.r1 |
| | | | | /CLONE = IMAGE: 31713 |
| | | | | /FEA = FLmRNA /CNT = 14 |
| | | | | /TID = Hs.13228.1 |
| | | | | /TIER = ConsEnd /STK = 0 |
| | | | | /UG = Hs.13228 /LL = 30818 |
| | | | | /UG_GENE = CSEN |
| | | | | /UG_TITLE = Calsenilin, |
| | | | | presenilin-binding protein, EF |
| | | | | hand transcription factor |
| | | | | /FL = gb: NM_013434.1 |
| | | | | gb: AF199599.1 |
| | | | | gb: AF120102.1 |
| 237249_at | −2.09 | KCNQ1 | Underexpressed | gb: AI970466 |
| | | | | /DB_XREF = gi: 5767292 |
| | | | | /DB_XREF = wq93b06.x1 |
| | | | | /CLONE = IMAGE: 2479571 |
| | | | | /FEA = EST /CNT = 8 |
| | | | | /TID = Hs.95162.0 |
| | | | | /TIER = ConsEnd /STK = 5 |
| | | | | /UG = Hs.95162 /LL = 10984 |
| | | | | /UG_GENE = KCNQ1OT1 |
| | | | | /UG_TITLE = KCNQ1 |
| | | | | overlapping transcript 1 |
| 242517_at | −2.9 | KISS1R | Underexpressed | gb: AI819198 |
| | | | | /DB_XREF = gi: 5438277 |
| | | | | /DB_XREF = wj32f02.x1 |
| | | | | /CLONE = IMAGE: 2404539 |
| | | | | /FEA = EST /CNT = 4 |
| | | | | /TID = Hs.208229.0 |
| | | | | /TIER = ConsEnd /STK = 3 |
| | | | | /UG = Hs.208229 |
| | | | | /UG_TITLE = ESTs |
| 206785_s_at | −2.86 | KLRC2 | Underexpressed | gb: NM_002260.2 |
| | | | | /DB_XREF = gi: 7108353 |
| | | | | /GEN = KLRC2 |
| | | | | /FEA = FLmRNA /CNT = 12 |
| | | | | /TID = Hs.177605.0 /TIER = FL |
| | | | | /STK = 1 /UG = Hs.177605 |
| | | | | /LL = 3822 /DEF = Homo |
| | | | | sapiens killer cell lectin-like |
| | | | | receptor subfamily C, member |
| | | | | 2 (KLRC2), mRNA. |
| | | | | /PROD = killer cell lectin-like |
| | | | | receptor subfamily C, member |
| | | | | 2 /FL = gb: NM_002260.2 |
| | | | | gb: AF260134.1 |
| 209351_at | −2.85 | KRT14 | Underexpressed | gb: BC002690.1 |
| | | | | /DB_XREF = gi: 12803708 |
| | | | | /FEA = FLmRNA /CNT = 212 |
| | | | | /TID = Hs.117729.0 |
| | | | | /TIER = FL + Stack /STK = 53 |
| | | | | /UG = Hs.117729 /LL = 3861 |
| | | | | /UG_GENE = KRT14 |
| | | | | /DEF = Homo sapiens, keratin |
| | | | | 14 (epidermolysis bullosa |
| | | | | simplex, Dowling-Meara, |
| | | | | Koebner), clone MGC: 3944, |
| | | | | mRNA, complete cds. |
| | | | | /PROD = keratin 14 |
| | | | | (epidermolysis bullosa |
| | | | | simplex, Dowling-Meara, |
| | | | | Koebner) |
| | | | | /FL = gb: NM_000526.1 |
| | | | | gb: BC002690.1 |
| 235871_at | −2.91 | LIPH | Underexpressed | gb: AA088857 |
| | | | | /DB_XREF = gi: 1634387 |
| | | | | /DB_XREF = z190f03.s1 |
| | | | | /CLONE = IMAGE: 511901 |
| | | | | /FEA = EST /CNT = 14 |
| | | | | /TID = Hs.68864.0 |
| | | | | /TIER = ConsEnd /STK = 2 |
| | | | | /UG = Hs.68864 |
| | | | | /UG_TITLE = ESTs, Weakly |
| | | | | similar to phosphatidylserine- |
| | | | | specific phospholipase A1 |
| | | | | deltaC (H. sapiens) |
| 219759_at | −6.02 | LRAP | Underexpressed | gb: NM_022350.1 |
| | | | | /DB_XREF = gi: 11641260 |
| | | | | /GEN = LOC64167 |
| | | | | /FEA = FLmRNA /CNT = 18 |
| | | | | /TID = Hs.280380.0 /TIER = FL |
| | | | | /STK = 0 /UG = Hs.280380 |
| | | | | /LL = 64167 /DEF = Homo |
| | | | | sapiens aminopeptidase |
| | | | | (LOC64167), mRNA. |
| | | | | /PROD = aminopeptidase |
| | | | | /FL = gb: AF191545.1 |
| | | | | gb: NM_022350.1 |
| 211018_at | −2.14 | LSS | Underexpressed | gb: D63807.1 |
| | | | | /DB_XREF = gi: 1019365 |
| | | | | /FEA = FLmRNA /CNT = 3 |
| | | | | /TID = Hs.93199.1 /TIER = FL |
| | | | | /STK = 0 /UG = Hs.93199 |
| | | | | /LL = 4047 /UG_GENE = LSS |
| | | | | /DEF = Human mRNA for |
| | | | | lanosterol synthase, complete |
| | | | | cds. /PROD = lanosterol |
| | | | | synthase /FL = gb: D63807.1 |
| 232819_s_at | −2.81 | LTBR | Underexpressed | gb: L04489.1 |
| | | | | /DB_XREF = gi: 340022 |
| | | | | /FEA = mRNA /CNT = 4 |
| | | | | /TID = Hs.117847.0 |
| | | | | /TIER = ConsEnd /STK = 0 |
| | | | | /UG = Hs.117847 |
| | | | | /UG_TITLE = Homo sapiens |
| | | | | (clone NCD18) tumor |
| | | | | necrosis factor receptor |
| | | | | related protein mRNA, |
| | | | | complete exon and repeat |
| | | | | region /DEF = Homo sapiens |
| | | | | (clone NCD18) tumor |
| | | | | necrosis factor receptor |
| | | | | related protein mRNA, |
| | | | | complete exon and repeat |
| | | | | region. |
| 204041_at | −2.67 | MAOB | Underexpressed | gb: NM_000898.1 |
| | | | | /DB_XREF = gi: 4505092 |
| | | | | /GEN = MAOB |
| | | | | /FEA = FLmRNA /CNT = 79 |
| | | | | /TID = Hs.82163.0 |
| | | | | /TIER = FL + Stack /STK = 39 |
| | | | | /UG = Hs.82163 /LL = 4129 |
| | | | | /DEF = Homo sapiens |
| | | | | monoamine oxidase B |
| | | | | (MAOB), nuclear gene |
| | | | | encoding mitochondrial |
| | | | | protein, mRNA. |
| | | | | /PROD = monoamine oxidase |
| | | | | B /FL = gb: NM_000898.1 |
| | | | | gb: M69177.1 |
| 226023_at | 2.69 | MAP2K7 | Overexpressed | gb: AI344194 |
| | | | | /DB_XREF = gi: 4081400 |
| | | | | /DB_XREF = tc02e10.x1 |
| | | | | /CLONE = IMAGE: 2062698 |
| | | | | /FEA = EST /CNT = 63 |
| | | | | /TID = Hs.296234.0 |
| | | | | /TIER = Stack /STK = 11 |
| | | | | /UG = Hs.296234 |
| | | | | /UG_TITLE = ESTs |
| 230848_s_at | 3.22 | MGA (includes | Overexpressed | gb: BF438227 |
| | | EG: 23269) | | /DB_XREF = gi: 11450744 |
| | | | | /DB_XREF = 7q68g05.x1 |
| | | | | /CLONE = IMAGE: 3703497 |
| | | | | /FEA = EST /CNT = 10 |
| | | | | /TID = Hs.23763.1 |
| | | | | /TIER = Stack /STK = 8 |
| | | | | /UG = Hs.23763 /LL = 23269 |
| | | | | /UG_GENE = KIAA0518 |
| | | | | /UG_TITLE = Max-interacting |
| | | | | protein |
| 244523_at | −2.12 | MMD | Underexpressed | gb: AW104453 |
| | | | | /DB_XREF = gi: 6075188 |
| | | | | /DB_XREF = xd78b02.x1 |
| | | | | /CLONE = IMAGE: 2603691 |
| | | | | /FEA = EST /CNT = 7 |
| | | | | /TID = Hs.99734.0 |
| | | | | /TIER = ConsEnd /STK = 1 |
| | | | | /UG = Hs.99734 |
| | | | | /UG_TITLE = ESTs |
| 204580_at | −6.06 | MMP12 | Underexpressed | gb: NM_002426.1 |
| | | | | /DB_XREF = gi: 4505206 |
| | | | | /GEN = MMP12 |
| | | | | /FEA = FLmRNA /CNT = 72 |
| | | | | /TID = Hs.1695.0 |
| | | | | /TIER = FL + Stack /STK = 18 |
| | | | | /UG = Hs.1695 /LL = 4321 |
| | | | | /DEF = Homo sapiens matrix |
| | | | | metalloproteinase 12 |
| | | | | (macrophage elastase) |
| | | | | (MMP12), mRNA. |
| | | | | /PROD = matrix |
| | | | | metalloproteinase 12 |
| | | | | preproprotein |
| | | | | /FL = gb: L23808.1 |
| | | | | gb: NM_002426.1 |
| 208423_s_at | 2.44 | MSR1 | Overexpressed | gb: NM_002445.1 |
| | | | | /DB_XREF = gi: 4505258 |
| | | | | /GEN = MSR1 |
| | | | | /FEA = FLmRNA /CNT = 2 |
| | | | | /TID = Hs.49.0 /TIER = FL |
| | | | | /STK = 0 /UG = Hs.49 |
| | | | | /LL = 4481 /DEF = Homo |
| | | | | sapiens macrophage scavenger |
| | | | | receptor 1 (MSR1), mRNA. |
| | | | | /PROD = macrophage |
| | | | | scavenger receptor 1 |
| | | | | /FL = gb: NM_002445.1 |
| 217109_at | −2.93 | MUC4 | Underexpressed | gb: AJ242547.1 |
| | | | | /DB_XREF = gi: 7406614 |
| | | | | /GEN = MUC4 /FEA = mRNA |
| | | | | /CNT = 2 /TID = Hs.198267.2 |
| | | | | /TIER = ConsEnd /STK = 0 |
| | | | | /UG = Hs.198267 /LL = 4585 |
| | | | | /DEF = Homo sapiens partial |
| | | | | mRNA for sv7-MUC4 |
| | | | | apomucin. /PROD = sv7- |
| | | | | MUC4 apomucin |
| 244149_at | 2.13 | MYL9 | Overexpressed | gb: AA864758 |
| | | | | /DB_XREF = gi: 2959071 |
| | | | | /DB_XREF = oh37g07.s1 |
| | | | | /CLONE = IMAGE: 1460028 |
| | | | | /FEA = EST /CNT = 5 |
| | | | | /TID = Hs.134192.0 |
| | | | | /TIER = ConsEnd /STK = 3 |
| | | | | /UG = Hs.134192 |
| | | | | /UG_TITLE = ESTs |
| 206022_at | −3.79 | NDP | Underexpressed | gb: NM_000266.1 |
| | | | | /DB_XREF = gi: 4557788 |
| | | | | /GEN = NDP /FEA = FLmRNA |
| | | | | /CNT = 28 /TID = Hs.2839.0 |
| | | | | /TIER = FL /STK = 0 |
| | | | | /UG = Hs.2839 /LL = 4693 |
| | | | | /DEF = Homo sapiens Norrie |
| | | | | disease (pseudoglioma) |
| | | | | (NDP), mRNA. |
| | | | | /PROD = Norrie disease protein |
| | | | | /FL = gb: NM_000266.1 |
| 239450_at | −2.18 | NDUFV2 | Underexpressed | gb: AA846867 |
| | | | | /DB_XREF = gi: 2933007 |
| | | | | /DB_XREF = aj42d09.s1 |
| | | | | /CLONE = IMAGE: 1392977 |
| | | | | /FEA = EST /CNT = 4 |
| | | | | /TID = Hs.116929.0 |
| | | | | /TIER = ConsEnd /STK = 4 |
| | | | | /UG = Hs.116929 |
| | | | | /UG_TITLE = ESTs |
| 242267_x_at | 2.23 | NINJ2 | Overexpressed | gb: T68304 |
| | | | | /DB_XREF = gi: 679452 |
| | | | | /DB_XREF = yc41e01.s1 |
| | | | | /CLONE = IMAGE: 83256 |
| | | | | /FEA = EST /CNT = 3 |
| | | | | /TID = Hs.182402.0 |
| | | | | /TIER = ConsEnd /STK = 3 |
| | | | | /UG = Hs.182402 |
| | | | | /UG_TITLE = ESTs |
| 206578_at | 2.69 | NKX2-5 | Overexpressed | gb: NM_004387.1 |
| | | | | /DB_XREF = gi: 4758089 |
| | | | | /GEN = CSX /FEA = FLmRNA |
| | | | | /CNT = 15 /TID = Hs.54473.0 |
| | | | | /TIER = FL /STK = 0 |
| | | | | /UG = Hs.54473 /LL = 1482 |
| | | | | /DEF = Homo sapiens cardiac- |
| | | | | specific homeo box (CSX), |
| | | | | mRNA. /PROD = cardiac- |
| | | | | specific homeo box |
| | | | | /FL = gb: NM_004387.1 |
| | | | | gb: U34962.1 gb: AB021133.1 |
| 243738_at | 2.22 | NMNAT3 | Overexpressed | gb: AW172570 |
| | | (includes | | /DB_XREF = gi: 6438518 |
| | | EG: 349565) | | /DB_XREF = xj79g02.x1 |
| | | | | /CLONE = IMAGE: 2663474 |
| | | | | /FEA = EST /CNT = 4 |
| | | | | /TID = Hs.130246.0 |
| | | | | /TIER = ConsEnd /STK = 3 |
| | | | | /UG = Hs.130246 |
| | | | | /UG_TITLE = ESTs |
| 231798_at | −5.28 | NOG | Underexpressed | gb: AL575177 |
| | | | | /DB_XREF = gi: 12936088 |
| | | | | /DB_XREF = AL575177 |
| | | | | /CLONE = CS0DI063YJ07 (3 |
| | | | | prime) /FEA = FLmRNA |
| | | | | /CNT = 7 /TID = Hs.248201.0 |
| | | | | /TIER = ConsEnd /STK = 1 |
| | | | | /UG = Hs.248201 /LL = 9241 |
| | | | | /UG_GENE = NOG |
| | | | | /UG_TITLE = noggin |
| | | | | /FL = gb: NM_005450.1 |
| 227816_at | −3.56 | NTN1 | Underexpressed | gb: BF591483 |
| | | | | /DB_XREF = gi: 11683807 |
| | | | | /DB_XREF = nab98c06.x1 |
| | | | | /CLONE = IMAGE: 3275890 |
| | | | | /FEA = EST /CNT = 23 |
| | | | | /TID = Hs.96917.0 |
| | | | | /TIER = Stack /STK = 15 |
| | | | | /UG = Hs.96917 |
| | | | | /UG_TITLE = ESTs |
| 206563_s_at | 6.14 | OPRL1 | Overexpressed | gb: AF348323.1 |
| | | | | /DB_XREF = gi: 13022242 |
| | | | | /GEN = ORL1 |
| | | | | /FEA = FLmRNA /CNT = 15 |
| | | | | /TID = Hs.2859.0 /TIER = FL |
| | | | | /STK = 0 /UG = Hs.2859 |
| | | | | /LL = 4987 /DEF = Homo |
| | | | | sapiens nociceptin receptor |
| | | | | (ORL1) mRNA, complete cds. |
| | | | | /PROD = nociceptin receptor |
| | | | | /FL = gb: NM_000913.1 |
| | | | | gb: U30185.1 gb: AF348323.1 |
| 1553319_at | −2.88 | OXGR1 | Underexpressed | gb: AF370886.1 |
| | | | | /DB_XREF = gi: 21728283 |
| | | | | /GEN = GPR99 |
| | | | | /TID = Hs2.352218.1 /CNT = 4 |
| | | | | /FEA = FLmRNA /TIER = FL |
| | | | | /STK = 1 /LL = 27199 |
| | | | | /UG = Hs.352218 /DEF = Homo |
| | | | | sapiens G protein-coupled |
| | | | | receptor GPR99 (GPR99) |
| | | | | mRNA, complete cds. |
| | | | | /PROD = G protein-coupled |
| | | | | receptor GPR99 |
| | | | | /FL = gb: AF370886.1 |
| | | | | gb: NM_080818.1 |
| 228703_at | −2.21 | P4HA3 | Underexpressed | gb: AW665086 |
| | | | | /DB_XREF = gi: 7457631 |
| | | | | /DB_XREF = hi99b06.x1 |
| | | | | /CLONE = IMAGE: 2980403 |
| | | | | /FEA = EST /CNT = 21 |
| | | | | /TID = Hs.179891.0 |
| | | | | /TIER = Stack /STK = 14 |
| | | | | /UG = Hs.179891 |
| | | | | /UG_TITLE = ESTs, Weakly |
| | | | | similar to P4HA_HUMAN |
| | | | | PROLYL 4- |
| | | | | HYDROXYLASE ALPHA |
| | | | | SUBUNIT PRECURSOR |
| | | | | (H. sapiens) |
| 221286_s_at | 3.08 | PACAP | Overexpressed | gb: NM_016459.1 |
| | | | | /DB_XREF = gi: 7706002 |
| | | | | /GEN = LOC51237 |
| | | | | /FEA = FLmRNA /CNT = 10 |
| | | | | /TID = Hs.122492.1 /TIER = FL |
| | | | | /STK = 1 /UG = Hs.122492 |
| | | | | /LL = 51237 /DEF = Homo |
| | | | | sapiens hypothetical protein |
| | | | | (LOC51237), mRNA. |
| | | | | /PROD = hypothetical protein |
| | | | | /FL = gb: NM_016459.1 |
| 206859_s_at | −10.02 | PAEP | Underexpressed | gb: NM_002571.1 |
| | | | | /DB_XREF = gi: 4505582 |
| | | | | /GEN = PAEP |
| | | | | /FEA = FLmRNA /CNT = 18 |
| | | | | /TID = Hs.82269.0 |
| | | | | /TIER = FL + Stack /STK = 13 |
| | | | | /UG = Hs.82269 /LL = 5047 |
| | | | | /DEF = Homo sapiens |
| | | | | progestagen-associated |
| | | | | endometrial protein (placental |
| | | | | protein 14, pregnancy- |
| | | | | associated endometrial alpha- |
| | | | | 2-globulin, alpha uterine |
| | | | | protein) (PAEP), mRNA. |
| | | | | /PROD = progestagen- |
| | | | | associated endometrial |
| | | | | protein(placental protein 14, |
| | | | | pregnancy-associated |
| | | | | endometrialalpha-2-globulin, |
| | | | | alpha uterine protein) |
| | | | | /FL = gb: NM_002571.1 |
| | | | | gb: J04129.1 |
| 211818_s_at | −3.42 | PDE4C | Underexpressed | gb: U88712.1 |
| | | | | /DB_XREF = gi: 1857983 |
| | | | | /FEA = FLmRNA /CNT = 1 |
| | | | | /TID = Hs.189.2 /TIER = FL |
| | | | | /STK = 0 /UG = Hs.189 |
| | | | | /LL = 5143 |
| | | | | /UG_GENE = PDE4C |
| | | | | /DEF = Human |
| | | | | phosphodiesterase 4C mRNA, |
| | | | | complete cds. |
| | | | | /PROD = phosphodiesterase 4C |
| | | | | /FL = gb: U88712.1 |
| 205593_s_at | 2 | PDE9A | Overexpressed | gb: NM_002606.1 |
| | | | | /DB_XREF = gi: 4505674 |
| | | | | /GEN = PDE9A |
| | | | | /FEA = FLmRNA /CNT = 44 |
| | | | | /TID = Hs.18953.0 |
| | | | | /TIER = FL + Stack /STK = 10 |
| | | | | /UG = Hs.18953 /LL = 5152 |
| | | | | /DEF = Homo sapiens |
| | | | | phosphodiesterase 9A |
| | | | | (PDE9A), mRNA. |
| | | | | /PROD = phosphodiesterase 9A |
| | | | | /FL = gb: AF048837.1 |
| | | | | gb: AF067223.1 |
| | | | | gb: NM_002606.1 |
| 1568949_at | −2.03 | PITPNC1 | Underexpressed | gb: BM042439 |
| | | | | /DB_XREF = gi: 16771706 |
| | | | | /DB_XREF = 603616266F1 |
| | | | | /CLONE = IMAGE: 5556809 |
| | | | | /TID = Hs2.397451.1 /CNT = 33 |
| | | | | /FEA = mRNA |
| | | | | /TIER = ConsEnd /STK = 0 |
| | | | | /UG = Hs.397451 |
| | | | | /UG_TITLE = Homo sapiens, |
| | | | | clone IMAGE: 3875257, |
| | | | | mRNA |
| 219014_at | −3.08 | PLAC8 | Underexpressed | gb: NM_016619.1 |
| | | | | /DB_XREF = gi: 7706157 |
| | | | | /GEN = LOC51316 |
| | | | | /FEA = FLmRNA /CNT = 86 |
| | | | | /TID = Hs.107139.0 /TIER = FL |
| | | | | /STK = 0 /UG = Hs.107139 |
| | | | | /LL = 51316 /DEF = Homo |
| | | | | sapiens hypothetical protein |
| | | | | (LOC51316), mRNA. |
| | | | | /PROD = hypothetical protein |
| | | | | /FL = gb: NM_016619.1 |
| | | | | gb: AF208846.1 |
| 228964_at | −2.75 | PRDM1 | Underexpressed | gb: AI692659 |
| | | | | /DB_XREF = gi: 4969999 |
| | | | | /DB_XREF = wd86d10.x1 |
| | | | | /CLONE = IMAGE: 2338483 |
| | | | | /FEA = EST /CNT = 22 |
| | | | | /TID = Hs.289088.2 |
| | | | | /TIER = Stack /STK = 18 |
| | | | | /UG = Hs.289088 /LL = 3320 |
| | | | | /UG_GENE = HSPCA |
| | | | | /UG_TITLE = heat shock 90 kD |
| | | | | protein 1, alpha |
| 211743_s_at | −4.67 | PRG2 (includes | Underexpressed | gb: BC005929.1 |
| | | EG: 5553) | | /DB_XREF = gi: 13543541 |
| | | | | /FEA = FLmRNA /CNT = 1 |
| | | | | /TID = HsAffx.900770.968 |
| | | | | /TIER = FL /STK = 0 |
| | | | | /DEF = Homo sapiens, |
| | | | | proteoglycan 2, bone marrow |
| | | | | (natural killer cell activator, |
| | | | | eosinophil granule major basic |
| | | | | protein), clone MGC: 14537, |
| | | | | mRNA, complete cds. |
| | | | | /PROD = proteoglycan 2, bone |
| | | | | marrow (natural killer |
| | | | | cellactivator, eosinophil |
| | | | | granule major basic protein) |
| | | | | /FL = gb: BC005929.1 |
| 205445_at | −7.86 | PRL | Underexpressed | gb: NM_000948.1 |
| | | | | /DB_XREF = gi: 4506104 |
| | | | | /GEN = PRL /FEA = FLmRNA |
| | | | | /CNT = 352 /TID = Hs.1905.0 |
| | | | | /TIER = FL + Stack /STK = 19 |
| | | | | /UG = Hs.1905 /LL = 5617 |
| | | | | /DEF = Homo sapiens prolactin |
| | | | | (PRL), mRNA. |
| | | | | /PROD = prolactin |
| | | | | /FL = gb: NM_000948.1 |
| 237234_at | −2.83 | PSCD2 | Underexpressed | gb: AA700869 |
| | | | | /DB_XREF = gi: 2704034 |
| | | | | /DB_XREF = zj36c04.s1 |
| | | | | /CLONE = IMAGE: 452358 |
| | | | | /FEA = EST /CNT = 6 |
| | | | | /TID = Hs.119761.0 |
| | | | | /TIER = ConsEnd /STK = 6 |
| | | | | /UG = Hs.119761 |
| | | | | /UG_TITLE = ESTs |
| 204945_at | 2.47 | PTPRN | Overexpressed | gb: NM_002846.1 |
| | | | | /DB_XREF = gi: 4506320 |
| | | | | /GEN = PTPRN |
| | | | | /FEA = FLmRNA /CNT = 76 |
| | | | | /TID = Hs.89655.0 |
| | | | | /TIER = FL + Stack /STK = 20 |
| | | | | /UG = Hs.89655 /LL = 5798 |
| | | | | /DEF = Homo sapiens protein |
| | | | | tyrosine phosphatase, receptor |
| | | | | type, N (PTPRN), mRNA. |
| | | | | /PROD = protein tyrosine |
| | | | | phosphatase, receptor type, N |
| | | | | /FL = gb: L18983.1 |
| | | | | gb: NM_002846.1 |
| 210823_s_at | −2.29 | PTPRS(includes | Underexpressed | gb: U40317.1 |
| | | EG: | | /DB_XREF = gi: 1407624 |
| | | | | /GEN = PTPsigma |
| | | | | /FEA = FLmRNA /CNT = 2 |
| | | | | /TID = Hs.159534.1 /TIER = FL |
| | | | | /STK = 0 /UG = Hs.159534 |
| | | | | /LL = 5802 /DEF = Human |
| | | | | protein tyrosine phosphatase |
| | | | | PTPsigma (PTPsigma) |
| | | | | mRNA, complete cds. |
| | | | | /PROD = PTPsigma |
| | | | | /FL = gb: U40317.1 |
| 235059_at | −4.54 | RAB12 | Underexpressed | gb: BF574430 |
| | | | | /DB_XREF = gi: 11648142 |
| | | | | /DB_XREF = 602131648F1 |
| | | | | /CLONE = IMAGE: 4271098 |
| | | | | /FEA = EST /CNT = 24 |
| | | | | /TID = Hs.29739.0 |
| | | | | /TIER = ConsEnd /STK = 0 |
| | | | | /UG = Hs.29739 |
| | | | | /UG_TITLE = ESTs, Weakly |
| | | | | similar to C34323 GTP- |
| | | | | binding protein Rab3A |
| | | | | (H. sapiens) |
| 211904_x_at | 2.01 | RAD52 | Overexpressed | gb: AF125950.1 |
| | | | | /DB_XREF = gi: 4581009 |
| | | | | /GEN = RAD52 |
| | | | | /FEA = FLmRNA /CNT = 2 |
| | | | | /TID = Hs.89571.3 /TIER = FL |
| | | | | /STK = 0 /UG = Hs.89571 |
| | | | | /LL = 5893 /DEF = Homo |
| | | | | sapiens DNA repair protein |
| | | | | RAD52 beta isoform |
| | | | | (RAD52) mRNA, |
| | | | | alternatively spliced, complete |
| | | | | cds. /PROD = DNA repair |
| | | | | protein RAD52 beta isoform |
| | | | | /FL = gb: AF125950.1 |
| 219140_s_at | −3.94 | RBP4 | Underexpressed | gb: NM_006744.2 |
| | | | | /DB_XREF = gi: 8400727 |
| | | | | /GEN = RBP4 /FEA = FLmRNA |
| | | | | /CNT = 153 /TID = Hs.76461.0 |
| | | | | /TIER = FL + Stack /STK = 50 |
| | | | | /UG = Hs.76461 /LL = 5950 |
| | | | | /DEF = Homo sapiens retinol- |
| | | | | binding protein 4, interstitial |
| | | | | (RBP4), mRNA. |
| | | | | /PROD = retinol-binding |
| | | | | protein 4, interstitialprecursor |
| | | | | /FL = gb: NM_006744.2 |
| | | | | gb: AF119868.1 |
| 204535_s_at | 2.34 | REST | Overexpressed | gb: AI978576 |
| | | | | /DB_XREF = gi: 5803606 |
| | | | | /DB_XREF = wq72c11.x1 |
| | | | | /CLONE = IMAGE: 2476820 |
| | | | | /FEA = FLmRNA /CNT = 60 |
| | | | | /TID = Hs.227630.0 |
| | | | | /TIER = Stack /STK = 9 |
| | | | | /UG = Hs.227630 /LL = 5978 |
| | | | | /UG_GENE = REST |
| | | | | /UG_TITLE = RE1-silencing |
| | | | | transcription factor |
| | | | | /FL = gb: U22680.1 |
| | | | | gb: U22314.1 |
| | | | | gb: NM_005612.1 |
| 210586_x_at | −6.11 | RHD | Underexpressed | gb: AF312679.1 |
| | | | | /DB_XREF = gi: 11878260 |
| | | | | /GEN = RHD /FEA = FLmRNA |
| | | | | /CNT = 20 /TID = Hs.278994.0 |
| | | | | /TIER = FL /STK = 0 |
| | | | | /UG = Hs.278994 /LL = 6006 |
| | | | | /DEF = Homo sapiens rhesus D |
| | | | | category VI type IV protein |
| | | | | (RHD) mRNA, complete cds. |
| | | | | /PROD = rhesus D category VI |
| | | | | type IV protein |
| | | | | /FL = gb: AF056965.1 |
| | | | | gb: AB018644.1 |
| | | | | gb: AB018645.1 |
| | | | | gb: NM_020485.1 |
| | | | | gb: M34015.1 gb: AB049754.1 |
| | | | | gb: AB049753.1 |
| | | | | gb: AF312679.1 |
| | | | | gb: AB030388.1 |
| 231040_at | −13.24 | RORB | Underexpressed | gb: AW512988 |
| | | | | /DB_XREF = gi: 7151066 |
| | | | | /DB_XREF = xt76b02.x1 |
| | | | | /CLONE = IMAGE: 2792331 |
| | | | | /FEA = EST /CNT = 9 |
| | | | | /TID = Hs.184780.0 |
| | | | | /TIER = Stack /STK = 9 |
| | | | | /UG = Hs.184780 |
| | | | | /UG_TITLE = ESTs |
| 205863_at | 2.7 | S100A12 | Overexpressed | gb: NM_005621.1 |
| | | | | /DB_XREF = gi: 5032058 |
| | | | | /GEN = S100A12 |
| | | | | /FEA = FLmRNA /CNT = 34 |
| | | | | /TID = Hs.19413.0 |
| | | | | /TIER = FL + Stack /STK = 12 |
| | | | | /UG = Hs.19413 /LL = 6283 |
| | | | | /DEF = Homo sapiens S100 |
| | | | | calcium-binding protein A12 |
| | | | | (calgranulin C) (S100A12), |
| | | | | mRNA. /PROD = S100 |
| | | | | calcium-binding protein A12 |
| | | | | /FL = gb: D83664.1 |
| | | | | gb: D49549.1 |
| | | | | gb: NM_005621.1 |
| 202917_s_at | 3.2 | S100A8 | Overexpressed | gb: NM_002964.2 |
| | | | | /DB_XREF = gi: 9845519 |
| | | | | /GEN = S100A8 |
| | | | | /FEA = FLmRNA /CNT = 257 |
| | | | | /TID = Hs.100000.0 |
| | | | | /TIER = FL + Stack /STK = 93 |
| | | | | /UG = Hs.100000 /LL = 6279 |
| | | | | /DEF = Homo sapiens S100 |
| | | | | calcium-binding protein A8 |
| | | | | (calgranulin A) (S100A8), |
| | | | | mRNA. /PROD = S100 |
| | | | | calcium-binding protein A8 |
| | | | | /FL = gb: NM_002964.2 |
| 230077_at | −2.05 | SDHAL1 | Underexpressed | gb: W90764 |
| | | | | /DB_XREF = gi: 1406730 |
| | | | | /DB_XREF = zh79h02.s1 |
| | | | | /CLONE = IMAGE: 418323 |
| | | | | /FEA = EST /CNT = 14 |
| | | | | /TID = Hs.469.1 /TIER = Stack |
| | | | | /STK = 8 /UG = Hs.469 |
| | | | | /LL = 6389 |
| | | | | /UG_GENE = SDHA |
| | | | | /UG_TITLE = succinate |
| | | | | dehydrogenase complex, |
| | | | | subunit A, flavoprotein (Fp) |
| 203789_s_at | −2.15 | SEMA3C | Underexpressed | gb: NM_006379.1 |
| | | | | /DB_XREF = gi: 5454047 |
| | | | | /GEN = SEMA3C |
| | | | | /FEA = FLmRNA /CNT = 92 |
| | | | | /TID = Hs.171921.0 |
| | | | | /TIER = FL + Stack /STK = 18 |
| | | | | /UG = Hs.171921 /LL = 10512 |
| | | | | /DEF = Homo sapiens sema |
| | | | | domain, immunoglobulin |
| | | | | domain (Ig), short basic |
| | | | | domain, secreted, |
| | | | | (semaphorin) 3C (SEMA3C), |
| | | | | mRNA. /PROD = sema |
| | | | | domain, immunoglobulin |
| | | | | domain (Ig), shortbasic |
| | | | | domain, secreted, |
| | | | | (semaphorin) 3C |
| | | | | /FL = gb: NM_006379.1 |
| | | | | gb: AB000220.1 |
| 202376_at | −12.84 | SERPINA3 | Underexpressed | gb: NM_001085.2 |
| | | | | /DB_XREF = gi: 9665246 |
| | | | | /GEN = SERPINA3 |
| | | | | /FEA = FLmRNA /CNT = 230 |
| | | | | /TID = Hs.234726.0 |
| | | | | /TIER = FL + Stack /STK = 128 |
| | | | | /UG = Hs.234726 /LL = 12 |
| | | | | /DEF = Home sapiens serine |
| | | | | (or cysteine) proteinase |
| | | | | inhibitor, clade A (alpha-1 |
| | | | | antiproteinase, antitrypsin), |
| | | | | member 3 (SERPINA3), |
| | | | | mRNA. /PROD = alpha-1- |
| | | | | antichymotrypsin, precursor |
| | | | | /FL = gb: NM_001085.2 |
| | | | | gb: BC003559.1 gb: K01500.1 |
| 207873_x_at | 3.13 | SEZ6L | Overexpressed | gb: NM_021115.1 |
| | | | | /DB_XREF = gi: 10863914 |
| | | | | /GEN = SEZ6L |
| | | | | /FEA = FLmRNA /CNT = 2 |
| | | | | /TID = Hs.194766.0 /TIER = FL |
| | | | | /STK = 0 /UG = Hs.194766 |
| | | | | /LL = 23544 /DEF = Homo |
| | | | | sapiens seizure related gene 6 |
| | | | | (mouse)-like (SEZ6L), |
| | | | | mRNA. /PROD = seizure |
| | | | | related gene 6 (mouse)-like |
| | | | | /FL = gb: NM_021115.1 |
| 238273_at | −2.45 | SLC13A4 | Underexpressed | gb: AW007221 |
| | | | | /DB_XREF = gi: 5855999 |
| | | | | /DB_XREF = ws50f07.x1 |
| | | | | /CLONE = IMAGE: 2500645 |
| | | | | /FEA = EST /CNT = 5 |
| | | | | /TID = Hs.200022.0 |
| | | | | /TIER = ConsEnd /STK = 5 |
| | | | | /UG = Hs.200022 |
| | | | | /UG_TITLE = ESTs |
| 228844_at | 2.78 | SLC13A5 | Overexpressed | gb: AI797218 |
| | | | | /DB_XREF = gi: 5362690 |
| | | | | /DB_XREF = we86a02.x1 |
| | | | | /CLONE = IMAGE: 2347946 |
| | | | | /FEA = EST /CNT = 40 |
| | | | | /TID = Hs.11860.0 |
| | | | | /TIER = Stack /STK = 17 |
| | | | | /UG = Hs.11860 |
| | | | | /UG_TITLE = ESTs |
| 202498_s_at | −2.33 | SLC2A3 | Underexpressed | gb: BE550486 |
| | | | | /DB_XREF = gi: 9792178 |
| | | | | /DB_XREF = 7a27c01.x1 |
| | | | | /CLONE = IMAGE: 3219936 |
| | | | | /FEA = FLmRNA /CNT = 182 |
| | | | | /TID = Hs.7594.0 /TIER = Stack |
| | | | | /STK = 8 /UG = Hs.7594 |
| | | | | /LL = 6515 |
| | | | | /UG_GENE = SLC2A3 |
| | | | | /UG_TITLE = solute carrier |
| | | | | family 2 (facilitated glucose |
| | | | | transporter), member 3 |
| | | | | /FL = gb: NM_006931.1 |
| | | | | gb: M20681.1 |
| 219911_s_at | −3.23 | SLCO4A1 | Underexpressed | gb: NM_016354.1 |
| | | | | /DB_XREF = gi: 7706516 |
| | | | | /GEN = SLC21A12 |
| | | | | /FEA = FLmRNA /CNT = 19 |
| | | | | /TID = Hs.235782.0 /TIER = FL |
| | | | | /STK = 1 /UG = Hs.235782 |
| | | | | /LL = 28231 /DEF = Homo |
| | | | | sapiens solute carrier family |
| | | | | 21 (organic anion transporter), |
| | | | | member 12 (SLC21A12), |
| | | | | mRNA. /PROD = organic |
| | | | | anion transporter OATP-E |
| | | | | /FL = gb: AB031051.1 |
| | | | | gb: NM_016354.1 |
| | | | | gb: AF205072.1 |
| | | | | gb: AF187817.1 |
| 214404_x_at | 2.13 | SPDEF | Overexpressed | gb: AI435670 |
| | | | | /DB_XREF = gi: 4304809 |
| | | | | /DB_XREF = th53b09.x1 |
| | | | | /CLONE = IMAGE: 2121977 |
| | | | | /FEA = EST /CNT = 18 |
| | | | | /TID = Hs.79414.2 |
| | | | | /TIER = Stack /STK = 18 |
| | | | | /UG = Hs.79414 /LL = 25803 |
| | | | | /UG_GENE = PDEF |
| | | | | /UG_TITLE = prostate |
| | | | | epithelium-specific Ets |
| | | | | transcription factor |
| 216965_x_at | −2.56 | SPG20 | Underexpressed | gb: AL139377 |
| | | | | /DB_XREF = gi: 10185469 |
| | | | | /FEA = mRNA /CNT = 2 |
| | | | | /TID = Hs.118087.2 |
| | | | | /TIER = ConsEnd /STK = 0 |
| | | | | /UG = Hs.118087 /LL = 23111 |
| | | | | /UG_GENE = KIAA0610 |
| | | | | /UG_TITLE = KIAA0610 |
| | | | | protein /DEF = Human DNA |
| | | | | sequence from clone RP11- |
| | | | | 251J8 on chromosome 13 |
| | | | | Contains ESTs, STSs, GSSs |
| | | | | and a CpG island. Contains |
| | | | | two novel genes with two |
| | | | | isoforms each and the |
| | | | | KIAA0610 gene with two |
| | | | | isoforms |
| 202363_at | −5.51 | SPOCK1 | Underexpressed | gb: AF231124.1 |
| | | | | /DB_XREF = gi: 7248844 |
| | | | | /FEA = FLmRNA /CNT = 190 |
| | | | | /TID = Hs.93029.0 |
| | | | | /TIER = FL + Stack /STK = 68 |
| | | | | /UG = Hs.93029 /LL = 6695 |
| | | | | /UG_GENE = SPOCK |
| | | | | /DEF = Homo sapiens testican- |
| | | | | 1 mRNA, complete cds. |
| | | | | /PROD = testican-1 |
| | | | | /FL = gb: NM_04598.1 |
| | | | | gb: AF231124.1 |
| 208539_x_at | 2.25 | SPRR2B | Overexpressed | gb: NM_006945.1 |
| | | | | /DB_XREF = gi: 5902119 |
| | | | | /GEN = SPRR2B |
| | | | | /FEA = FLmRNA /CNT = 2 |
| | | | | /TID = Hs.231622.0 /TIER = FL |
| | | | | /STK = 0 /UG = Hs.231622 |
| | | | | /LL = 6701 /DEF = Homo |
| | | | | sapiens small proline-rich |
| | | | | protein 2B (SPRR2B), |
| | | | | mRNA. /PROD = small |
| | | | | proline-rich protein 2B |
| | | | | /FL = gb: NM_006945.1 |
| 202400_s_at | 2.35 | SRF | Overexpressed | gb: AI188786 |
| | | | | /DB_XREF = gi: 3739995 |
| | | | | /DB_XREF = qd16g09.x1 |
| | | | | /CLONE = IMAGE: 1723936 |
| | | | | /FEA = FLmRNA /CNT = 153 |
| | | | | /TID = Hs.155321.0 |
| | | | | /TIER = Stack /STK = 18 |
| | | | | /UG = Hs.155321 /LL = 6722 |
| | | | | /UG_GENE = SRF |
| | | | | /UG_TITLE = serum response |
| | | | | factor (c-fos serum response |
| | | | | element-binding transcription |
| | | | | factor) /FL = gb: J03161.1 |
| | | | | gb: NM_003131.1 |
| 235591_at | −2.84 | SSTR1 | Underexpressed | gb: R62424 |
| | | | | /DB_XREF = gi: 834303 |
| | | | | /DB_XREF = yg52c10.s1 |
| | | | | /CLONE = IMAGE: 36221 |
| | | | | /FEA = EST /CNT = 11 |
| | | | | /TID = Hs.126059.0 |
| | | | | /TIER = ConsEnd /STK = 0 |
| | | | | /UG = Hs.126059 |
| | | | | /UG_TITLE = ESTs |
| 223285_s_at | −2.29 | ST6GALNAC4 | Underexpressed | gb: AW044319 |
| | | | | /DB_XREF = gi: 5904848 |
| | | | | /DB_XREF = wv67g10.x1 |
| | | | | /CLONE = IMAGE: 2534658 |
| | | | | /FEA = FLmRNA /CNT = 126 |
| | | | | /TID = Hs.3972.1 /TIER = Stack |
| | | | | /STK = 8 /UG = Hs.3972 |
| | | | | /LL = 27090 |
| | | | | /UG_GENE = ST6GALNACIV |
| | | | | /UG_TITLE = NeuAc-alpha- |
| | | | | 2,3-Gal-beta-1,3-GalNAc- |
| | | | | alpha-2,6-sialyltransferase |
| | | | | alpha2,6-sialyltransferase |
| | | | | /FL = gb: AB035172.1 |
| 1570420_at | 2.25 | STXBP2 | Overexpressed | gb: AF318317.1 |
| | | | | /DB_XREF = gi: 18027725 |
| | | | | /GEN = pp10122 |
| | | | | /TID = Hs2.398002.1 /CNT = 1 |
| | | | | /FEA = mRNA |
| | | | | /TIER = ConsEnd /STK = 0 |
| | | | | /UG = Hs.398002 /DEF = Homo |
| | | | | sapiens pp10122 mRNA, |
| | | | | complete cds. |
| | | | | /PROD = unknown |
| 204776_at | −3.94 | THBS4 | Underexpressed | gb: NM_003248.1 |
| | | | | /DB_XREF = gi: 4507488 |
| | | | | /GEN = THBS4 |
| | | | | /FEA = FLmRNA /CNT = 69 |
| | | | | /TID = Hs.75774.0 |
| | | | | /TIER = FL + Stack /STK = 37 |
| | | | | /UG = Hs.75774 /LL = 7060 |
| | | | | /DEF = Homo sapiens |
| | | | | thrombospondin 4 (THBS4), |
| | | | | mRNA. |
| | | | | /PROD = thrombospondin 4 |
| | | | | /FL = gb: NM_003248.1 |
| 238688_at | −2.37 | TPM1 | Underexpressed | gb: AI521618 |
| | | | | /DB_XREF = gi: 4435753 |
| | | | | /DB_XREF = to65f10.x1 |
| | | | | /CLONE = IMAGE: 2183179 |
| | | | | /FEA = EST /CNT = 9 |
| | | | | /TID = Hs.270915.0 |
| | | | | /TIER = ConsEnd /STK = 4 |
| | | | | /UG = Hs.270915 |
| | | | | /UG_TITLE = ESTs |
| 217143_s_at | −4.87 | TRA@ | Underexpressed | gb: X06557.1 |
| | | | | /DB_XREF = gi: 37003 |
| | | | | /FEA = mRNA /CNT = 2 |
| | | | | /TID = Hs.2014.2 |
| | | | | /TIER = ConsEnd /STK = 0 |
| | | | | /UG = Hs.2014 /LL = 6964 |
| | | | | /UG_GENE = TRD@ |
| | | | | /UG_TITLE = T cell receptor |
| | | | | delta locus /DEF = Human |
| | | | | mRNA for TCR-delta chain. |
| 215484_at | 2.26 | TRIM3 | Overexpressed | gb: AF070538.1 |
| | | | | /DB_XREF = gi: 3387895 |
| | | | | /FEA = mRNA /CNT = 10 |
| | | | | /TID = Hs.159408.0 |
| | | | | /TIER = ConsEnd /STK = 3 |
| | | | | /UG = Hs.159408 |
| | | | | /UG_TITLE = Homo sapiens |
| | | | | clone 24420 mRNA sequence |
| | | | | /DEF = Homo sapiens clone |
| | | | | 24420 mRNA sequence. |
| 1566598_at | 2.14 | UGT2B7 | Overexpressed | gb: AK094928.1 |
| | | | | /DB_XREF = gi: 21754091 |
| | | | | /TID = Hs2.269720.2 /CNT = 1 |
| | | | | /FEA = mRNA |
| | | | | /TIER = ConsEnd /STK = 0 |
| | | | | /UG = Hs.269720 |
| | | | | /UG_TITLE = Homo sapiens |
| | | | | cDNA: FLJ22384 fis, clone |
| | | | | HRC07594. /DEF = Homo |
| | | | | sapiens cDNA FLJ37609 fis, |
| | | | | clone BRCOC2011010. |
| 206213_at | 2.03 | WNT10B | Overexpressed | gb: NM_003394.1 |
| | | | | /DB_XREF = gi: 5803222 |
| | | | | /GEN = WNT10B |
| | | | | /FEA = FLmRNA /CNT = 24 |
| | | | | /TID = Hs.91985.0 |
| | | | | /TIER = FL + Stack /STK = 10 |
| | | | | /UG = Hs.91985 /LL = 7480 |
| | | | | /DEF = Homo sapiens |
| | | | | wingless-type MMTV |
| | | | | integration site family, |
| | | | | member 10B (WNT10B), |
| | | | | mRNA. /PROD = wingless- |
| | | | | type MMTV integration site |
| | | | | family, member 10B |
| | | | | /FL = gb: NM_003394.1 |
| | | | | gb: U81787.1 |
| 206067_s_at | −5.27 | WT1 | Underexpressed | gb: NM_024426.1 |
| | | | | /DB_XREF = gi: 13386509 |
| | | | | /GEN = WT1 /FEA = FLmRNA |
| | | | | /CNT = 24 /TID = Hs.1145.1 |
| | | | | /TIER = FL + Stack /STK = 11 |
| | | | | /UG = Hs.1145 /LL = 7490 |
| | | | | /DEF = Homo sapiens Wilms |
| | | | | tumor 1 (WT1), transcript |
| | | | | variant D, mRNA. |
| | | | | /PROD = Wilms tumor 1 |
| | | | | isoform D |
| | | | | /FL = gb: NM_024424.1 |
| | | | | gb: NM_024426.1 |
| 204148_s_at | 3.02 | ZP3 | Overexpressed | gb: NM_012230.1 |
| | | | | /DB_XREF = gi: 6912599 |
| | | | | /GEN = POMZP3 |
| | | | | /FEA = FLmRNA /CNT = 84 |
| | | | | /TID = Hs.296380.0 |
| | | | | /TIER = FL + Stack /STK = 32 |
| | | | | /UG = Hs.296380 /LL = 22932 |
| | | | | /DEF = Homo sapiens POM |
| | | | | (POM121 rat homolog) and |
| | | | | ZP3 fusion protein |
| | | | | (POMZP3), mRNA. |
| | | | | /PROD = POM (POM121 rat |
| | | | | homolog) and ZP3 fusion |
| | | | | protein /FL = gb: NM_012230.1 |
| | | | | gb: U10099.1 |

* Probe ID in Affymetrix HU133 Plus 2 GeneChips

TABLE 6
Hypoxia-regulated genes (Banu et al., 2006)
Oxygen Supply

| Probe ID* | Gene Symbol | FC** |
|---|---|---|
| 218120 | HO-2 | 1.09 |
| 218121 | HO-2 | 1.03 |
| 218995 | Endothelin-1 | 1.32 |
| 222802 | Endothelin-1 | 1.41 |
| 1564630 | Endothelin-1 | −1.30 |
| 202912 | Adrenomedullin | −1.16 |
| 207257 | EPO | 1.36 |
| 217254 | EPO | −1.23 |
| 203683 | VEGFA | −1.01 |
| 210512 | VEGFA | −1.19 |
| 210513 | VEGFA | −1.10 |
| 212171 | VEGFA | 1.04 |
| 203934 | Flk-1/KDR/VEGF- | 1.16 |
| | R2 | |
| 204406 | Flt-1/VEGF-R1 | 1.25 |
| 210287 | Flt-1/VEGF-R1 | −1.03 |
| 222033 | Flt-1/VEGF-R1 | 1.13 |
| 232809 | Flt-1/VEGF-R1 | −1.59 |
| 207092 | Leptin | −1.43 |
| 208921 | Tyrosine | −2.04 |
| | hydroxylase | |
| 201808 | Endoglin | −1.19 |
| 201809 | Endoglin | −1.01 |
| 228586 | Endoglin | 2.23 |

| | | |
|---|---|---|
| Probe ID | Gene Symbol | FC |

Cellular Metabolism

| | | |
|---|---|---|
| 202464 | PFKFB3 | −1.12 |
| 206246 | PFKFB4 | 1.56 |
| 228499 | PFKFB4 | 1.00 |
| 217398 | GAPDH | 1.11 |
| 212581 | GAPDH | 1.18 |
| 213453 | GAPDH | 1.08 |
| 210096 | CYP4B1 | −1.64 |
| 200650 | LDH A | 1.01 |

Cell Growth and
Apoptosis

| | | |
|---|---|---|
| 209201 | CXCR4 | 1.12 |
| 211919 | CXCR4 | 1.15 |
| 217028 | CXCR4 | 1.09 |
| 205302 | IGFBP-1 | −50.00 |
| 237989 | IGFBP-1 | −1.69 |
| 209101 | CTGF | −1.07 |
| 206067 | WT-1 | −5.26 |
| 216953 | WT-1 | −1.23 |
| 1555271 | TERT | −1.12 |
| 207199 | TERT | −1.61 |
| 211599 | met | −1.07 |
| 213816 | met | −1.20 |
| 213807 | met | −1.15 |

Others

| | | |
|---|---|---|
| 201945 | Furin | 1.24 |
| 220781 | DEC1 | −1.15 |
| 213931 | IDA2/ID2B | −1.28 |
| 207980 | Cited 2 | 1.05 |
| 209357 | Cited 2 | −1.06 |
| 227287 | Cited 2 | −1.22 |
| 160020 | MMP-14 | −1.05 |
| 202827 | MMP-14 | −1.11 |
| 202828 | MMP-14 | 1.08 |
| 217279 | MMP-14 | 1.21 |

*Probe ID in Affymetrix HU133 Plus 2 GeneChip
**FC = Fold Change ±2 considered significant

TABLE 7

| | | |
|---|---|---|
| Probe ID* | Gene Symbol | FC** |

Putative HIF-2α regulated genes (Liu et al., 2000)

| | | |
|---|---|---|
| 206702 | TIE-2/TEK tyrosine kinase | 1.15 |
| 217711 | TIE-2/TEK tyrosine kinase | −1.01 |
| 203934 | Flk-1/KDR/VEGF-R2 | 1.16 |
| 1562263 | Lysyl oxidase-like-2/LOXL2 | −1.61 |
| 202997 | Lysyl oxidase-like-2/LOXL2 | 1.10 |
| 202998 | Lysyl oxidase-like-2/LOXL2 | −1.05 |
| 202999 | Lysyl oxidase-like-2/LOXL2 | 1.20 |
| 204298 | Lysyl oxidase/LOX | −1.06 |
| 215446 | Lysyl oxidase/LOX | −1.12 |
| 209357 | CITED2 | −1.06 |
| 227287 | CITED2 | −1.22 |
| 207980 | CITED2 | 1.05 |
| 203683 | VEGFA | −1.01 |
| 210512 | VEGFA | −1.84 |
| 210513 | VEGFA | −1.10 |
| 212171 | VEGFA | 1.04 |
| 207257 | EPO | 1.36 |
| 217254 | EPO | −1.23 |
| 160020 | MMP-14 | −1.05 |
| 202827 | MMP-14 | −1.11 |
| 202828 | MMP-14 | 1.08 |
| 217279 | MMP-14 | 1.21 |
| 210265 | Oct-4 | −2.94 |
| 205015 | TGF-α | −2.78 |
| 211258 | TGF-α | 1.30 |

Oxidative stress-regulated genes (Schwaeble et al., 1991)

| | | |
|---|---|---|
| 200642 | SOD 1 soluble | −1.04 |
| 205236 | SOD 3 extracellular | 1.06 |
| 200800 | Heat shock 70 KDa protein | 1.03 |
| | HSPA1A///HSPA1B | |
| 201841 | Heat shock 27 Kda protein 7 | −1.06 |
| 202581 | Heat shock 70 Kda protein 1B | 1.11 |
| 203751 | JUN D proto-oncogene | 1.20 |
| 203752 | JUN D proto-oncogene | 1.08 |
| 214326 | JUN D proto-oncogene | 1.23 |
| 208864 | Thioredoxin | −1.04 |
| 216609 | Thioredoxin | 1.14 |
| 209077 | Thioredoxin 2 | 1.10 |
| 209078 | Thioredoxin 2 | −1.02 |
| 211300 | p53 | −1.05 |
| 201746 | p53 | 1.04 |
| 215078 | SOD 2 mitochondrial | 1.55 |
| 215223 | SOD 2 mitochondrial | −1.27 |
| 216841 | SOD 2 mitochondrial | −1.43 |
| 216336 | Metallothioein 1A///1M | −1.04 |
| 218120 | HO 2 | 1.09 |
| 218121 | HO 2 | 1.03 |

*Probe ID in Affymetrix HU133 Plus 2 Genechip
**FC = Fold Change ±2 considered significant

TABLE 8
J5 and Fold Change Genes that Secrete or Shed Proteins
(certain genes have both fold change and J5 values (bold))

| | | | | | Expression |
|---|---|---|---|---|---|
| GeneSym | GeneName | Compartment | ProbeID | FC_J5 | Profile |
| ACACA | acetyl-Coenzyme A | Cytoplasm | 214358_at | −2.12 | Underexpressed |
| | carboxylase alpha | | | | |
| AHSG | alpha-2-HS- | Extracellular | 210929_s_at | 2.39 | Overexpressed |
| | glycoprotein | Space | | | |
| ALDH1A2 | aldehyde | Cytoplasm | 207016_s_at | −7.83 | Underexpressed |
| | dehydrogenase 1 | | | | |
| | family, member A2 | | | | |
| ANXA13 | annexin A13 | Plasma | 208323_s_at | 2.89 | Overexpressed |
| | | Membrane | | | |
| APC | adenomatous polyposis | Nucleus | 203527_s_at | −2.58 | Underexpressed |
| | coli | | | | |
| ARFGEF2 | ADP-ribosylation | Cytoplasm | 239373_at | 3.41 | Overexpressed |
| | factor guanine | | | | |
| | nucleotide-exchange | | | | |
| | factor 2 (brefeldin A- | | | | |
| | inhibited) | | | | |
| ART1 | ADP- | Plasma | 207919_at | −2.2 | Underexpressed |
| | ribosyltransferase 1 | Membrane | | | |
| BDKRB2 | bradykinin receptor B2 | Plasma | 205870_at | −2.7 | Underexpressed |
| | | Membrane | | | |
| C4BPA | complement | Extracellular | 205654_at | −2.67 | Underexpressed |
| | component 4 binding | Space | | | |
| | protein, alpha | | | | |
| CCK | cholecystokinin | Extracellular | 205827_at | 4.46 | Overexpressed |
| | | Space | | | |
| CCK | cholecystokinin | Extracellular | 205827_at | 20.41 | Overexpressed |
| | | Space | | | |
| CD52 | CD52 molecule | Plasma | 34210_at | 2.42 | Overexpressed |
| | | Membrane | | | |
| CFHR1 | complement factor H- | Extracellular | 215388_s_at | −8.449 | Underexpressed |
| | related 1 | Space | | | |
| CEHR1 | complement factor H- | Extracellular | 215388_s_at | −3.48 | Underexpressed |
| | related 1 | Space | | | |
| COL5A1 | collagen, type V, alpha 1 | Extracellular | 213818_x_at | 2.12 | Overexpressed |
| | | Space | | | |
| CPM | carboxypeptidase M | Plasma | 235019_at | −2.06 | Underexpressed |
| | | Membrane | | | |
| CR1 | complement | Plasma | 217552_x_at | 2 | Overexpressed |
| | component (3b/4b) | Membrane | | | |
| | receptor 1 (Knops | | | | |
| | blood group) | | | | |
| CUL1 | cullin 1 | Nucleus | 243286_at | −2.01 | Underexpressed |
| CXCL9 | chemokine (C—X—C | Extracellular | 203915_at | 2.07 | Overexpressed |
| | motif) ligand 9 | Space | | | |
| DPYSL4 | dihydropyrimidinase- | Cytoplasm | 205493_s_at | 2.01 | Overexpressed |
| | like 4 | | | | |
| EGR1 | early growth response 1 | Nucleus | 227404_s_at | 2.37 | Overexpressed |
| EPAS1 | endothelial PAS | Nucleus | 242868_at | −15.342 | Underexpressed |
| | domain protein 1 | | | | |
| EPAS1 | endothelial PAS | Nucleus | 242868_at | −7.64 | Underexpressed |
| | domain protein 1 | | | | |
| F11R | F11 receptor | Plasma | 226482_s_at | −8.441 | Underexpressed |
| | | Membrane | | | |
| F2R | coagulation factor II | Plasma | 203989_x_at | −2.98 | Underexpressed |
| | (thrombin) receptor | Membrane | | | |
| FKBP1A | FK506 binding protein | Cytoplasm | 239248_at | 2.1 | Overexpressed |
| | 1A, 12 kDa | | | | |
| FLT4 | fms-related tyrosine | Plasma | 229902_at | −2.12 | Underexpressed |
| | kinase 4 | Membrane | | | |
| FN1 | fibronectin 1 | Plasma | 214702_at | −8.2 | Underexpressed |
| | | Membrane | | | |
| FN1 | fibronectin 1 | Plasma | 214702_at | −2.35 | Underexpressed |
| | | Membrane | | | |
| FOS | v-fos FBJ murine | Nucleus | 209189_at | 2.78 | Overexpressed |
| | osteosarcoma viral | | | | |
| | oncogene homolog | | | | |
| FOSB | FBJ murine | Nucleus | 202768_at | 2.33 | Overexpressed |
| | osteosarcoma viral | | | | |
| | oncogene homolog B | | | | |
| FPRL2 | formyl peptide | Plasma | 214560_at | 2 | Overexpressed |
| | receptor 3 | Membrane | | | |
| FSTL3 | follistatin-like 3 | Extracellular | 203592_s_at | −10.048 | Underexpressed |
| | (secreted glycoprotein) | Space | | | |
| FSTL3 | follistatin-like 3 | Extracellular | 203592_s_at | −2.56 | Underexpressed |
| | (secreted glycoprotein) | Space | | | |
| FUT6 | fucosyltransferase 6 | Cytoplasm | 1565661_x_at | 2.02 | Overexpressed |
| | (alpha (1,3) | | | | |
| | fucosyltransferase) | | | | |
| GATA1 | GATA binding protein | Nucleus | 1555590_a_at | −2.47 | Underexpressed |
| | 1 (globin transcription | | | | |
| | factor 1) | | | | |
| GNG4 | guanine nucleotide | Plasma | 1555765_a_at | −3.73 | Underexpressed |
| | binding protein (G | Membrane | | | |
| | protein), gamma 4 | | | | |
| GNG7 | guanine nucleotide | Plasma | 232043_at | −2.67 | Underexpressed |
| | binding protein (G | Membrane | | | |
| | protein), gamma 7 | | | | |
| GNLY | granulysin | Cytoplasm | 205495_s_at | −23.51 | Underexpressed |
| GTPBP2 | GTP binding protein 2 | Unknown | 223789_s_at | −2.17 | Underexpressed |
| GZMB | granzyme B | Cytoplasm | 210164_at | −2.7 | Underexpressed |
| | (granzyme 2, cytotoxic | | | | |
| | T-lymphocyte- | | | | |
| | associated serine | | | | |
| | esterase 1) | | | | |
| HBE1 | hemoglobin, epsilon 1 | Cytoplasm | 205919_at | −2.64 | Underexpressed |
| HBZ | hemoglobin, zeta | Cytoplasm | 206647_at | −6.51 | Underexpressed |
| HEXA | hexosaminidase A | Cytoplasm | 1556426_at | 2.46 | Overexpressed |
| | (alpha polypeptide) | | | | |
| HLA- | major | Plasma | 203290_at | 2.21 | Overexpressed |
| DQA1 | histocompatibility | Membrane | | | |
| | complex, class II, DQ | | | | |
| | alpha 1 | | | | |
| HTR2B | 5-hydroxytryptamine | Plasma | 206638_at | −2.51 | Underexpressed |
| | (serotonin) receptor 2B | Membrane | | | |
| IGFBP1 | insulin-like growth | Extracellular | 205302_at | −53.23 | Underexpressed |
| | factor binding protein 1 | Space | | | |
| IGEBP1 | insulin-like growth | Extracellular | 205302_at | −10.347 | Underexpressed |
| | factor binding protein 1 | Space | | | |
| IKZF1 | IKAROS family zinc | Nucleus | 205038_at | 2.06 | Overexpressed |
| | finger 1 (Ikaros) | | | | |
| IL15 | interleukin 15 | Extracellular | 205992_s_at | −2.76 | Underexpressed |
| | | Space | | | |
| IL1B | interleukin 1, beta | Extracellular | 205067_at | −2.14 | Underexpressed |
| | | Space | | | |
| IL1RL1 | interleukin 1 receptor- | Plasma | 207526_s_at | −2.93 | Underexpressed |
| | like 1 | Membrane | | | |
| IL2RB | interleukin 2 receptor, | Plasma | 205291_at | −2.77 | Underexpressed |
| | beta | Membrane | | | |
| KCNH2 | potassium voltage- | Plasma | 210036_s_at | −2.06 | Underexpressed |
| | gated channel, | Membrane | | | |
| | subfamily H (eag- | | | | |
| | related), member 2 | | | | |
| KCNIP3 | Kv channel interacting | Nucleus | 231774_at | −2.42 | Underexpressed |
| | protein 3, calsenilin | | | | |
| KLRC2 | killer cell lectin-like | Plasma | 206785_s_at | −2.86 | Underexpressed |
| | receptor subfamily C, | Membrane | | | |
| | member 2 | | | | |
| KRT14 | keratin 14 | Cytoplasm | 209351_at | −8.699 | Underexpressed |
| (includes | (epidermolysis bullosa | | | | |
| EG: 3861) | simplex, Dowling- | | | | |
| | Meara, Koebner) | | | | |
| KRT14 | keratin 14 | Cytoplasm | 209351_at | −2.85 | Underexpressed |
| (includes | (epidermolysis bullosa | | | | |
| EG: 3861) | simplex, Dowling- | | | | |
| | Meara, Koebner) | | | | |
| LIPH | lipase, member H | Extracellular | 235871_at | −2.91 | Underexpressed |
| | | Space | | | |
| LSS | lanosterol synthase | Cytoplasm | 211018_at | −2.14 | Underexpressed |
| | (2,3-oxidosqualene- | | | | |
| | lanosterol cyclase) | | | | |
| LTBR | lymphotoxin beta | Plasma | 232819_s_at | −2.81 | Underexpressed |
| | receptor (TNFR | Membrane | | | |
| | superfamily, member | | | | |
| | 3) | | | | |
| MAP2K7 | mitogen-activated | Cytoplasm | 226023_at | 2.69 | Overexpressed |
| | protein kinase kinase 7 | | | | |
| MMP12 | matrix | Extracellular | 204580_at | −17.183 | Underexpressed |
| | metallopeptidase 12 | Space | | | |
| | (macrophage elastase) | | | | |
| MMP12 | matrix | Extracellular | 204580_at | −6.06 | Underexpressed |
| | metallopeptidase 12 | Space | | | |
| | (macrophage elastase) | | | | |
| MSR1 | macrophage scavenger | Plasma | 208423_s_at | 2.44 | Overexpressed |
| | receptor 1 | Membrane | | | |
| MYL9 | myosin, light chain 9, | Cytoplasm | 244149_at | 2.13 | Overexpressed |
| (includes | regulatory | | | | |
| EG: 10398) | | | | | |
| NOG | noggin | Extracellular | 231798_at | −5.28 | Underexpressed |
| | | Space | | | |
| PAEP | progestagen-associated | Extracellular | 206859_s_at | −15.642 | Underexpressed |
| | endometrial protein | Space | | | |
| | (placental protein 14, | | | | |
| | pregnancy-associated | | | | |
| | endometrial alpha-2- | | | | |
| | globulin, alpha uterine | | | | |
| | protein) | | | | |
| PAEP | progestagen-associated | Extracellular | 206859_s_at | −10.02 | Underexpressed |
| | endometrial protein | Space | | | |
| | (placental protein 14, | | | | |
| | pregnancy-associated | | | | |
| | endometrial alpha-2- | | | | |
| | globulin, alpha uterine | | | | |
| | protein) | | | | |
| PDE4C | phosphodiesterase 4C, | Cytoplasm | 211818_s_at | −3.42 | Underexpressed |
| | cAMP-specific | | | | |
| | (phosphodiesterase E1 | | | | |
| | dunce homolog, | | | | |
| | Drosophila) | | | | |
| PRDM1 | PR domain containing | Nucleus | 228964_at | −2.75 | Underexpressed |
| | 1, with ZNF domain | | | | |
| PRG2 | proteoglycan 2, bone | Extracellular | 211743_s_at | −4.67 | Underexpressed |
| (includes | marrow (natural killer | Space | | | |
| EG: 5553) | cell activator, | | | | |
| | eosinophil granule | | | | |
| | major basic protein) | | | | |
| PRL | prolactin | Extracellular | 205445_at | −7.86 | Underexpressed |
| | | Space | | | |
| PTPRS | protein tyrosine | Plasma | 210823_s_at | −2.29 | Underexpressed |
| | phosphatase, receptor | Membrane | | | |
| | type, S | | | | |
| RBP4 | retinol binding protein | Extracellular | 219140_s_at | −3.94 | Underexpressed |
| | 4, plasma | Space | | | |
| RHD | Rh blood group, D | Plasma | 210586_x_at | −6.11 | Underexpressed |
| | antigen | Membrane | | | |
| S100A12 | S100 calcium binding | Cytoplasm | 205863_at | 2.7 | Overexpressed |
| | protein A12 | | | | |
| S100A8 | S100 calcium binding | Cytoplasm | 202917_s_at | 3.2 | Overexpressed |
| | protein A8 | | | | |
| S100A8 | S100 calcium binding | Cytoplasm | 202917_s_at | 13.185 | Overexpressed |
| | protein A8 | | | | |
| SART3 | squamous cell | Nucleus | 1554276_at | −13.803 | Underexpressed |
| | carcinoma antigen | | | | |
| | recognized by T cells 3 | | | | |
| SEMA3C | sema domain, | Extracellular | 203789_s_at | −8.419 | Underexpressed |
| | immunoglobulin | Space | | | |
| | domain (Ig), short basic | | | | |
| | domain, secreted, | | | | |
| | (semaphorin) 3C | | | | |
| SEMA3C | sema domain, | Extracellular | 203789_s_at | −2.15 | Underexpressed |
| | immunoglobulin | Space | | | |
| | domain (Ig), short basic | | | | |
| | domain, secreted, | | | | |
| | (semaphorin) 3C | | | | |
| SERPINA3 | serpin peptidase | Extracellular | 202376_at | −12.84 | Underexpressed |
| | inhibitor, clade A | Space | | | |
| | (alpha-1 antiproteinase, | | | | |
| | antitrypsin), member 3 | | | | |
| SLC16A6 | solute carrier family | Plasma | 230748_at | −8.374 | Underexpressed |
| | 16, member 6 | Membrane | | | |
| | (monocarboxylic acid | | | | |
| | transporter 7) | | | | |
| SLC2A3 | solute carrier family 2 | Plasma | 202498_s_at | −2.33 | Underexpressed |
| | (facilitated glucose | Membrane | | | |
| | transporter), member 3 | | | | |
| SRF | serum response factor | Nucleus | 202400_s_at | 2.35 | Overexpressed |
| | (c-fos serum response | | | | |
| | element-binding | | | | |
| | transcription factor) | | | | |
| SSTR1 | somatostatin receptor 1 | Plasma | 235591_at | −2.84 | Underexpressed |
| | | Membrane | | | |
| STXBP2 | syntaxin binding | Unknown | 1570420_at | 2.25 | Overexpressed |
| | protein 2 | | | | |
| THBS4 | thrombospondin 4 | Extracellular | 204776_at | −3.94 | Underexpressed |
| | | Space | | | |
| TPM1 | tropomyosin 1 (alpha) | Cytoplasm | 238688_at | −2.37 | Underexpressed |
| TRA@ | T cell receptor alpha | Cytoplasm | 217143_s_at | −4.87 | Underexpressed |
| | locus | | | | |

TABLE 9
Genes differentially expressed in first trimester PE

| | | | Gene Symbol | | Localization and |
|---|---|---|---|---|---|
| | | | & MIM | GO Biological | Function of Immune- |
| Probe Set ID | J5 Score | Gene Title | Number | Process/Description | related Genes |
| 205827_at | 20.41 | Cholecystokinin | CCK | Neuron migration /// | |
| | | | 118440 | signal transduction /// | |
| | | | | axonogenesis /// eating | |
| | | | | behavior | |
| 215141_at | 13.77 | chromosome 4 | C4orf10 | — | |
| | | open reading frame | | | |
| | | 10 | | | |
| 202917_s_at | 13.19 | S100 calcium | S100A8 | inflammatory response | Chorionic villi, |
| | | binding protein A8 | 123885 | | monocytes, |
| | | | | | macrophages, epithelial |
| | | | | | cells; chronic |
| | | | | | inflammation; cystic |
| | | | | | fibrosis antigen |
| | | | | | (1 related to HELLP) |
| 215733_x_at | 8.52 | cancer/testis | CTAG2 | biological_process | |
| | | antigen 2 | 300396 | | |
| 234601_x_at | 8.37 | CDNA: FLJ22732 | —* | — | |
| | | fis, clone HSI15880 | | | |
| 227238_at | −8.01 | mucin 15, cell | MUC15 | — | |
| | | surface associated | 608566 | | |
| 239010_at | −8.13 | Hypothetical gene | LOC440157 | — | |
| | | supported by | | | |
| | | AK096951; | | | |
| | | BC066547 | | | |
| 214702_at | −8.20 | fibronectin 1 | FN1 | acute-phase response /// | plasma, epithelial cell |
| | | | 135600 | cell adhesion /// | surface, extracellular |
| | | | | transmembrane receptor | matrix (ECM); |
| | | | | protein tyrosine kinase | migration stimulating |
| | | | | signaling pathway /// | factor, inflammation, |
| | | | | metabolism /// response | binds collagen, binds |
| | | | | to wounding /// cell | complement, stimulates |
| | | | | migration /// cell | endocytosis and |
| | | | | adhesion | clearance of particulate |
| | | | | | material from the |
| | | | | | circulation |
| 1553319_at | −8.25 | oxoglutarate (alpha- | OXGR1 | signal transduction /// | |
| | | ketoglutarate) | 606922 | G-protein coupled | |
| | | receptor 1 | | receptor protein | |
| | | | | signaling pathway /// G- | |
| | | | | protein coupled | |
| | | | | receptor protein | |
| | | | | signaling pathway | |
| 235592_at | −8.25 | Elongation factor, | ELL2 | transcription /// | |
| | | RNA polymerase | 601874 | regulation of | |
| | | II, 2 | | transcription, DNA- | |
| | | | | dependent /// RNA | |
| | | | | elongation from RNA | |
| | | | | polymerase II promoter | |
| 229839_at | −8.30 | Scavenger receptor | SCARA5 | phosphate transport | |
| | | class A, member 5 | 611306 | | |
| | | (putative) | | | |
| 230748_at | −8.37 | solute carrier | SLC16A6 | transport /// | |
| | | family 16, member | 603880 | monocarboxylic acid | |
| | | 6 (monocarboxylic | | transport | |
| | | acid transporter 7) | | | |
| 203789_s_at | −8.42 | sema domain, | SEMA3C | immune response /// | axons; |
| | | immunoglobulin | 602645 | transmembrane receptor | secreted Ig; |
| | | domain (Ig), short | | protein tyrosine kinase | integrin inhibition |
| | | basic domain, | | signaling pathway /// | |
| | | secreted, | | development /// | |
| | | (semaphorin) 3C | | response to drug | |
| 226482_s_at | −8.44 | F11 receptor | F11R | cell motility /// | tight junctions of |
| | | | 605721 | inflammatory response | epithelial and |
| | | | | /// cell adhesion /// | endothelial cells, |
| | | | | epithelial cell | hematopoietic cells; |
| | | | | differentiation | tight junction assembly |
| | | | | | and stabilization, |
| | | | | | leukocyte |
| | | | | | transmigration, and |
| | | | | | platelet activation |
| 215388_s_at | −8.45 | complement factor | CFH /// | complement activation, | liver, monocytes, |
| | | H /// complement | CFHR1 | alternative pathway /// | human umbilical vein |
| | | factor H-related 1 | 134370 /// | complement activation | endothelial cells |
| | | | 134371 /// | /// immune response /// | (HUVEC), fibroblasts, |
| | | | 235400 /// | innate immune response | placenta; |
| | | | 609814 /// | | alternative complement |
| | | | 610698 | | pathway in innate |
| | | | | | immune system |
| | | | | | (1 postpartum case |
| | | | | | study) |
| 1562053_at | −8.58 | CDNA clone | — | — | |
| | | IMAGE: 5267797 | | | |
| 219911_s_at | −8.59 | solute carrier | SLCO4A1 | ion transport /// | |
| | | organic anion | | transport | |
| | | transporter family, | | | |
| | | member 4A1 | | | |
| 209351_at | −8.70 | keratin 14 | KRT14 | cell morphogenesis /// | |
| | | (epidermolysis | 125595 /// | biological_process /// | |
| | | bullosa simplex, | 131800 /// | epidermis development | |
| | | Dowling-Meara, | 148066 /// | /// epidermis | |
| | | Koebner) | 161000 /// | development | |
| | | | 601001 | | |
| 1552858_at | −8.92 | melanoma antigen | MAGEB6 | biological_process | |
| | | family B, 6 | | | |
| | | 300467 | | | |
| 215108_x_at | −8.95 | trinucleotide repeat | TNRC9 | regulation of | |
| | | containing 9 | | transcription, DNA- | |
| | | 611416 | | dependent | |
| 226403_at | −8.97 | transmembrane | TMC4 | — | |
| | | channel-like 4 | | | |
| 207607_at | −9.23 | achaete-scute | ASCL2 | regulation of | |
| | | complex homolog 2 | 601886 | transcription, DNA- | |
| | | (Drosophila) | | dependent /// | |
| | | | | central nervous system | |
| | | | | development /// | |
| | | | | peripheral nervous | |
| | | | | system development /// | |
| | | | | cell differentiation /// | |
| | | | | regulation of | |
| | | | | transcription | |
| 228293_at | −9.41 | DEP domain | DEPDC7 | intracellular signaling | |
| | | containing 7 | | cascade /// | |
| | | | | biological_process | |
| 210251_s_at | −9.55 | RUN and FYVE | RUFY3 | — | |
| | | domain containing 3 | 611194 | | |
| 1561318_at | −9.69 | CDNA clone | — | — | |
| | | IMAGE: 5287025 | | | |
| 241036_at | −9.76 | Hermansky-Pudlak | HPS3 | organelle organization | kidney, liver placental |
| | | syndrome 3 | 606118 | and biogenesis | cells; |
| | | | | | lysosomal vesicles, |
| | | | | | platelet storage |
| | | | | | deficiency |
| 219759_at | −9.92 | leukocyte-derived | LRAP | proteolysis /// blood | endoplasmic reticulum; |
| | | arginine | 609497 | pressure regulation /// | maintenance of |
| | | aminopeptidase | | antigen processing and | homeostasis, |
| | | | | presentation of | maintenance of normal |
| | | | | endogenous peptide | pregnancy, memory |
| | | | | antigen via MHC class I | retention, blood |
| | | | | | pressure regulation, |
| | | | | | antigen presentation |
| 203592_s_at | −10.05 | follistatin-like 3 | FSTL3 | negative regulation of | |
| | | (secreted | 605343 | BMP signaling pathway | |
| | | glycoprotein) | | | |
| 205302_at | −10.35 | insulin-like growth | IGFBP1 | regulation of cell | liver, secretory |
| | | factor binding | 146730 | growth /// signal | endometrium, and |
| | | protein 1 | | transduction | decidua; |
| | | | | | decidualization and |
| | | | | | trophoblast invasion |
| 1568554_x_at | −10.67 | Chromosome 6 | C6orf142 | — | |
| | | open reading frame | | | |
| | | 142 | | | |
| 1554276_at | −13.80 | squamous cell | SART3 | RNA processing | RNA-binding nuclear |
| | | carcinoma antigen | 611684 | | protein; |
| | | recognized by T | | | tumor-rejection |
| | | cells 3 | | | antigen; induces HLA- |
| | | | | | A24-restricted and |
| | | | | | tumor-specific |
| | | | | | cytotoxic T |
| | | | | | lymphocytes in cancer |
| 242842_at | −14.51 | Transcribed locus | — | — | |
| 242868_at | −15.34 | Endothelial PAS | EPAS1 | angiogenesis /// | |
| | | domain protein 1 | 603349 | response to hypoxia /// | |
| | | | | regulation | |
| | | | | of transcription, DNA- | |
| | | | | dependent /// | |
| | | | | transcription | |
| | | | | from RNA polymerase | |
| | | | | II promoter /// signal | |
| | | | | transduction /// cell | |
| | | | | differentiation /// | |
| | | | | transcription /// signal | |
| | | | | transduction /// | |
| | | | | development /// | |
| | | | | regulation of | |
| | | | | transcription | |
| 206859_s_at | −15.64 | Progestagen- | PAEP | transport /// | genital tract, |
| | | associated | 173310 | development | secretory endometrium |
| | | endometrial protein | | | and decidua; |
| | | (placental protein | | | inhibits E-selectin- |
| | | 14, pregnancy- | | | mediated cell adhesion; |
| | | associated | | | neovascularization |
| | | endometrial alpha- | | | |
| | | 2-globulin, alpha | | | |
| | | uterine protein) | | | |
| 204580_at | −17.18 | Matrix | MMP12 | peptidoglycan | macrophages, ECM; |
| | | metallopeptidase 12 | 601046 | metabolism /// | Degrades elastin, tissue |
| | | (macrophage | | proteolysis /// | remodeling and repair |
| | | elastase) | | cell motility /// | during development |
| | | | | proteolysis | and inflammation |
| | | | | | (1 in vitro) |
| 207509_s_at | −18.87 | Leukocyte- | LAIR2 | — | homologous with |
| | | associated | 602993 | | LAIR1 on NK cells, T |
| | | immunoglobulin- | | | cells, B cells, |
| | | like receptor 2 | | | macrophages, and |
| | | | | | dendritic cells; |
| | | | | | secreted Ig-like |
| | | | | | receptor |

Table downloaded from Affymetrix.

### REFERENCES

Allen R, Tresini, M. Oxidative stress and gene regulation. Free Radic Biol Med 2000 28(3):463-99.
American College of Obstetricians and Gynecologists CoPB—O. ACOG practice bulletin. Diagnosis and management of preeclampsia and eclampsia. Number 33, January 2002. Obstet Gynecol 2002; 99(1):159-67.
Apps R, Gardner, L, Sharkey, A M, Holmes, N, Moffett, A. A homodimeric complex of HLA-G on normal trophoblast cells modulates antigen-presenting cells via LILRB1. Eur J Immunol 2007 37(7):1924-37.
Bagnard D, Lohrum, M, Uziel, D, Pu\ddot{}schel, A W, Bolz J. Semaphorins act as attractive and repulsive guidance signals during the development of cortical projections. Development 1998; 125:5043-53.
Banu N, Teichman, J, Dunlap-Brown, M, Villegas, G, Tufro, A. Semaphorin 3C regulates endothelial cell function by increasing integrin activity. FASEB J 2006 20(12):2150-2.
Bazzoni G. The jam family of junctional adhesion molecules. Curr Opin Cell Biol 2003; 15(5):525-30.
Benirschke K, Kaufmann, P, Baergen, R (Eds.). Pathology of the human placenta, 5th ed. ed. New York: Springer; 2006.
Bing D H, Almeda, S., Isliker, H., Lahav, J., Hynes, R. O. Fibronectin binds to the C1q component of complement. Proc Nat Acad Sci 1982; 79:4198-201.
Bischof P. Three pregnancy proteins (PP12, PP14, and PAPP-A): their biological and clinical relevance. Am J Perinatol 1989; 6(2):110-6.
Buimer M, Keijser, R, Jebbink, J M, Wehkamp, D, van Kampen, A H, Boer, K, van der Post, J A, Ris-Stalpers, C. Seven placental transcripts characterize HELLP-syndrome Placenta 2008; March 26 [Epub ahead of print].
Chappell S, Morgan, L. Searching for genetic clues to the causes of pre-eclampsia. Clin Sci (Lond) 2006; 110(4):443-58.
Christiansen L, Collins, K A. Pregnancy-associated deaths: a 15-year retrospective study and overall review of maternal pathophysiology. Am J Forensic Med Pathol 2006 27(1):11-9.
Damsky C, Fitzgerald, M L, Fisher, S J Distribution patterns of extracellular matrix components and adhesion receptors are intricately modulated during first trimester cytotrophoblast differentiation along the invasive pathway, in vivo. J Clin Invest 1992; 89:210-22.
Dharmaraj S. RT-PCR: The basics. 2007(Apr. 2, 2008).
Founds S, Dorman, J S, & Conley, Y P. Microarray technology applied to the complex disorder of preeclampsia. J Obstet Gynecol Neonatal Nurs 2008; 37(2):146-57.
Gifford R, August, P A, Cunningham, G, Green, L A, Lindheimer, M D, McNellis, D, Roberts, J M, Sibai, B M, Taler, S J. Report of the National High Blood Pressure Education Program Working Group on High Blood Pressure in Pregnancy. Am J Obstet Gynecol 2000; 183:S1-S22.
Guleria I, Pollard, J W. The trophoblast is a component of the innate immune system during pregnancy. Nat Med 2000; 6(5):589-93.
Huppertz B. The feto-maternal interface: setting the stage for potential immune interactions. Semin Immunopathol2007; 29(2):83-94.
Ilekis J, Reddy, U M, Roberts, J M. Preeclampsia—a pressing problem: an executive summary of a National Institute of Child Health and Human Development workshop. Reprod Sci 2007; 14(6):508-23.
Ingenuity. Ingenuity Pathways Analysis 5.5. In: Ingenuity Systems®; 2007.
Irving J, Lala, P K Functional role of cell surface integrinson human trophoblast cell migration: regulation by TGF-beta, IGF-II, and IGFBP-1. Exp Cell Res 1995; 217:419-27.
Jauniaux E, Watson, A L, Hempstock, J, Bao, Y P, Skepper, J N, Burton, B J. Onset of maternal arterial blood flow and placental oxidative stress. Am J Pathol 2000:157:2111-22.
Jeschke U, Kunert-Keil, C, Mylonas, I, Hammer, A, Schiessl, B, Lomba, I, Kuhn, C, Schulze, S, Friese, K. Expression of glycodelin A in decidual tissue of preeclamptic, HELLP and intrauterine growth-restricted pregnancies. Virchows Arch 2005; 446(4):360-8.
Jones R L S L, Zhao Y C, Ethier J F, Drummond A E, Findlay J K. Expression of activin receptors, follistatin and betaglycan by human endometrial stromal cells; consistent with a role for activins during decidualization. Mol Hum Reprod 2002; 8(4):363-74.
Khong T, De Wolf, F, Robertson, W B, Brosens, I. Inadequate maternal vascular response to placentation in pregnancies complicated by pre-eclampsia and by small-for-gestational age infants. Br J Obstet Gynaecol 1986; 93:1049-59.
Lessin D, Hunt, J S, King, C R, Wood, G W. Antigen expression by cells near the maternal-fetal interface. Am J Reprod Immunol Microbiol 1988; 16(1):1-7.
Lethe B, Lucas, S, Michaux, L, De Smet, C, Godelaine, D, Serrano, A, De Plaen, E, Boon, T. LAGE-1, a new gene with tumor specificity. Int J Cancer 1998; 76:903-8.
Li C, Hung Wong, W. Model-based analysis of oligonucleotide arrays: model validation, design issues and standard error application. Genome Biol 2001; 2(8).
Liu Y, Nusrat, A, Schnell, FJ, Reaves, T. A, Walsh, S, Pochet, M, Parkos, C A. Human junction adhesion molecule regulates tight junction resealing in epithelia. J Cell Sci 2000; 113:2363-74.
Löfstedt T, Fredlund, E, Holmquist-Mengelbier, L, Pietras, A, Ovenberger, M, Poellinger, L, Påhlman, S. Hypoxia inducible factor-2alpha in cancer. Cell Cycle 2007; 6(8):919-26.
Many A, Hubei, C A, Fisher, S J, Roberts, J M, Zhou, Y. Invasive cytotrophoblasts manifest evidence of oxidative stress in preeclampsia. Am J Pathol 2000; 156:321-31.
Mincheva-Nilsson L N O, Sundqvist K G, Hammarstrom M L, Hammarstrom S, Baranov V. gammadelta T cells of human early pregnancy decidua: evidence for cytotoxic potency. Int Immunol 2000 12(5):585-96.
Mohaupt M. Molecular aspects of preeclampsia. Mol Aspects Med 2007; 28(2):169-91.
Mor G A, V M Trophoblast cells as immune regulators In: Mor G, ed. Immunology of pregnancy. New York: Springer Science+Business Media; 2006:215-28.
NetAffx. 2007. (Accessed Oct. 10, 2007, at https://www.affymetrix.com/analysis/netaffx/)
Nishizawa H, Pryor-Koishi, K, Kato, T, Kowa, H, Kurahashi, H, Udagawa, Y. Microarray analysis of differentially expressed fetal genes in placental tissue derived from early and late onset severe pre-eclampsia. Placenta 2007; 28(5-6):487-97.
Patel S L-W, J. A web application for the integrated analysis of global gene expression patterns in cancer. Applied Bioinformatics 2004; 3:49-62.
Petty H, Kindzelskii, A L, Espinoza, J, Romero, R. Trophoblast contact deactivates human neutrophils. J Immunol 2006 176(5):3205-14.
Pijnenborg R, Bland, J M, Robertson, W B, Brosens, I. Uteroplacental arterial changes related to interstitial trophoblast migration in early human pregnancy. Placenta 1983 4(4):397-413.

Rajakumar A B H, Daftary A, Ness R, Conrad K P. Evidence for the functional activity of hypoxia-inducible transcription factors overexpressed in preeclamptic placentae. Placenta 2004; 25(10):763-9.
Rajakumar A, Jeyabalan, A, Markovic, N, Ness, R, Gilmour, C, Conrad, K P. Placental HIF-1 alpha, HIF-2 alpha, membrane and soluble VEGF receptor-1 proteins are not increased in normotensive pregnancies complicated by late-onset intrauterine growth restriction. Am J Physiol Regul Integr Comp Physiol 2007:R766-74.
Rajakumar A, Whitelock, K A, Daftary, A R, Markovic, N, Conrad, K P. Selective overexpression of the hypoxia inducible transcription factor, HIF-2α, in the placenta from women with preeclampsia. Biol Reprod 2001; 64:91-8 (error: 499-506).
Redman C, Sacks, G P, Sargent, I L. Preeclampsia: an excessive maternal inflammatory response to pregnancy. Am J Obstet Gynecol 1999; 180(2 Pt 1):499-506.
Redman C, Sargent, I L. Latest advances in understanding preeclampsia. Science 2005; 308(5728):1592-4.
Reimer T, Koczan, D, Gerber, B, Richter, D, Thiesen, H J, Friese, K. Microarray analysis of differentially expressed genes in placental tissue of pre-eclampsia: up-regulation of obesity-related genes. Mol Hum Reprod 2002; 8(7):674-80.
Roberts J, Lain, K Y. Recent insights into the pathogenesis of pre-eclampsia. Placenta 2002; 23:359-72.
Roberts J, Pearson, G, Cutler, J, Lindheimer, M, NHLBI Working Group. Summary of the NHLBI Working Group on research on hypertension during pregnancy. Hypertension 2003; 41(3):437-45.
Roberts J. Redman, C W G. Pre-eclampsia: More than pregnancy-induced hypertension. Lancet 1993; 341(1):447-1451.
Roberts J C, D W. Pathogenesis and genetics of pre-eclampsia. Lancet 2001; 357:53-6.
Sakai T, Larsen, M, Yamada, K M. Fibronectin requirement in branching morphogenesis. Nature Genet 2003; 423:876-81.
Sato N, Isono, K, Ishiwata, I, Nakai, M, Kami, K. Tissue expression of the S100 protein family-related MRP8 gene in human chorionic villi by in situ hybridization techniques. Okajimas Folia Anat Jpn 1999; 76((2-3):123-9.
Schwaeble W, Schwaiger, H, Brooimans, R A, Barbieri, A, Most, J, Hirsch-Kauffmann, M, Tiefenthaler, M, Lappin, D F, Daha, M R, Whaley, K, et al. Human complement factor H. Tissue specificity in the expression of three different mRNA species. Eur J Biochem 1991; 198(2):399-404.
Shyu M, Lin, M C, Shih, J C, Lee, C N, Huang, J, Liao C H, Huang, I F, Chen, H Y, Huang, M C, Hsieh, F J. Mucin 15 is expressed in human placenta and suppresses invasion of trophoblast-like cells in vitro. Hum Reprod 2007; 22(10):2723-32.
Sibai B, Dekker, G, Kupferminc, M. Pre-eclampsia. Lancet 2005; 365(9461):785-99.
Simon R, Lam, R A, Li, M-C, Ngan, M, Menenzes, S, & Zhao, Y. Analysis of Gene Expression Data Using BRB-Array Tools Cancer. Informatics 2007; 2:11-7.
Sorg C. The calcium binding proteins MRP8 and MRP14 in acute and chronic inflammation. Behring Inst Mitt 1992 91:126-37.
Stella C, Sibai, B M. Preeclampsia: Diagnosis and management of the atypical presentation. J Matern Fetal Neonatal Med 2006; 19(7):381-6.
Tazuke S I M N, Sugawara J, Carland G, Faessen G H, Suen L F, Irwin J C, Powell D R, Giaccia A J, Giudice L C. Hypoxia stimulates insulin-like growth factor binding protein 1 (IGFBP-1) gene expression in HepG2 cells: a possible model for IGFBP-1 expression in fetal hypoxia. Proc Natl Acad Sci USA 1998; 95(17):10188-93.
Telgmann R G B. Marker genes of decidualization: activation of the decidual prolactin gene. Hum Reprod Update 1998; 4(5):472-9.
Wenger R, Stiehl, D P, Camenisch, G. Integration of oxygen signaling at the consensus HRE. Sci STKE 2005; 2005(306):re12.

We claim:

1. A method for diagnosing, monitoring, or predicting preeclampsia in a pregnant woman, the method comprising the steps of:
(i) quantitatively determining the amount of one or more nucleic acid species in a biological sample obtained from the pregnant woman, wherein the RNA species hybridize with probe set ID Nos: 205827_at; 215141_at; 202917_s_at; 215733_x_at; 234601_x_at; 227238_at; 239010_at; 214702_at; 1553319_at; 235592_at; 229839_at; 230748_at; 203789_s_at; 226482_s_at; 215388_s_at; 1562053_at; 219911_s_at; 209351_at; 1552858_at; 215108_x_at; 226403_at; 207607_at; 228293_at; 210251_s_at; 1561318_at; 241036_at; 219759_at; 203592_s_at; 205302_at; 1568554_x_at; 1554276_at; 242842_at; 242868_at; 206859_s_at; 204580_at; and 207509_s_at or those set forth in Table 5 and wherein the biological sample is chorionic villus; and
(ii) comparing the amount of the nucleic acid species from step (i) to a standard control representing the amount of the nucleic acid species in the corresponding sample from an average non-preeclamptic pregnant woman, wherein an increase or a decrease in the amount of the nucleic acid species from the standard control indicates preeclampsia or an increased risk of developing preeclampsia.

2. The method of claim 1, wherein step (i) comprises using a reverse transcriptase polymerase chain reaction (RT-PCR).

3. The method of claim 1, wherein step (i) further comprises using mass spectrometry following RT-PCR.

4. The method of claim 1, wherein step (i) comprises using a polynucleotide hybridization method.

5. The method of claim 1, wherein step (i) comprises using a primer extension reaction.

6. The method of claim 1, wherein the woman is during the first trimester of gestation.

7. The method of claim 1, wherein the woman is during the second or third trimester of gestation.

8. The method of claim 1, wherein the increase in the amount of nucleic acid in the sample from the pregnant woman varies from a standard control by more than 2.00 fold.

9. The method of claim 1, wherein the decrease in the amount of nucleic acid from the pregnant woman varies from a standard control by more than 2.00 fold.

10. A kit for diagnosing, monitoring, or predicting preeclampsia in a pregnant woman, the kit comprising:
(i) PCR primers for quantitatively determining the amount of one or more nucleic acid species in a biological sample obtained from the pregnant woman, wherein the PCR primers correspond to the nucleic acid sequences of probe set ID Nos: 205827_at; 215141_at; 202917_s_at; 215733_x_at; 234601_x_at; 227238_at; 239010_at; 214702_at; 1553319_at; 235592 at; 229839_at; 230748 at; 203789 s_at; 226482 s_at; 215388_s_at; 1562053_at; 219911_s_at; 209351_at; 1552858_at; 215108_x_at; 226403_at; 207607_at; 228293_at; 210251_s_at; 1561318 at; 241036 at; 219759_at; 203592_s_at; 205302_at; 1568554_x_at; 1554276_at; 242842_at; 242868_at; 206859_s_at; 204580_at; and 207509_s_at on the AFFYMETRIX GeneChip system (Affymetrix, Santa Clara, Calif.; HG-U133 Plus 2.0 GeneChips containing 53,613 probe sets), the nucleic acid sequences set forth in Table 5 or combinations of said PCR primers; and
(ii) a standard control representing the amount of the nucleic acid species in the corresponding sample from an average non-preeclamptic pregnant woman.

11. An immunoassay-based method for diagnosing, monitoring, or predicting preeclampsia in a pregnant woman, the method comprising the steps of:
(i) binding one or more polypeptide species in a biological sample obtained from the pregnant woman to an antibody and quantitatively determining the amount of said one or more polypeptide species in said biological sample, wherein the said one or more polypeptide species is selected from those disclosed in Table 8; and
(ii) comparing the amount of the polypeptide species from step (i) to a standard control representing the amount of the polypeptide species in the corresponding sample from an average non-preeclamptic pregnant woman, wherein an increase or a decrease in the amount of the polypeptide species from the standard control indicates preeclampsia or an increased risk of developing preeclampsia.

12. The method of claim 11, wherein the woman is during the first trimester of gestation.

13. The method of claim 11, wherein the woman is during the second or third trimester of gestation.

14. The method of claim 1, wherein the increase in the amount of polypeptide is overexpressed or underexpressed in the sample from the pregnant woman as compared to a standard control.

15. The method according to claim 11, wherein the biological sample is blood, serum, plasma, endometrial tissue, washing from the reproductive tract, urine, saliva, cerebral spinal fluid, amniotic fluid, or chorionic villus.

16. The method according to claim 11, wherein said immunoassay-based method is a radioimmunoassay, ELISA (enzyme-linked immunosorbant assay), sandwich immunoassay, immunoradiometric assay or Western blot.
